(19) United States
(12) Patent Application Publication
(10) Pub. No.: US 2024/0309384 A1
(43) Pub. Date: Sep. 19, 2024

(54) INVERTED CHIMERIC siRNA MOLECULES AND METHODS OF USE THEREOF

(71) Applicant: The University of North Carolina at Chapel Hill, Chapel Hill, NC (US)

(72) Inventors: Chad Pecot, Pittsboro, NC (US); Yogitha Chareddy, Chapel Hill, NC (US)

(21) Appl. No.: 18/571,985

(22) PCT Filed: Jun. 21, 2022
(86) PCT No.: PCT/US2022/034235

§ 371 Date: Dec. 19, 2023

Related U.S. Application Data
(60) Provisional application No. 63/212,976, filed on Jun. 21, 2021.

(51) Int. Cl. C12N 15/113; A61P 31/14

(52) U.S. Cl. CPC C12N 15/1135; A61P 31/14; C12N 2310/14

(57) ABSTRACT

The invention relates to inverted chimera siRNA molecules and their use for the inhibition of expression of one or more target genes. The invention further relates to the inhibition of expression of c-Myc, or the dual inhibition of c-Myc and KRAS, using RNA interference, chemically-modified oligonucleotides, and/or chimeric siRNA multivalent combinations.

### STATEMENT OF PRIORITY

This application claims the benefit of U.S. Provisional Application Ser. No. 63/212,976, filed Jun. 21, 2021, the entire contents of which are incorporated by reference herein.

### FIELD OF THE INVENTION

The invention relates to inverted chimera siRNA molecules and their use for the inhibition of expression of one or more target genes. The invention further relates to the inhibition of expression of c-Myc, or the dual inhibition of c-Myc and KRAS, using RNA interference, chemically-modified oligonucleotides, and/or chimeric siRNA multivalent combinations.

### BACKGROUND OF THE INVENTION

The c-Myc protein is well-characterized as an oncogenic driver of aggressive cancer types but has largely been considered “undruggable” due to its intracellular localization, ubiquitous tissue expression. and lack of an enzymatic binding site. Similarly, the RAS genes encode a family of small GTPases that act upon downstream effector proteins to promote cell survival, growth, and proliferation (Khosravi-Far et al., Cancer Metastasis Rev. 13:67 (1994)). Proper function of the RAS proteins relies upon activation via a guanine nucleotide exchange factor (GEF) to its active, GTP-bound form as well as membrane association of the RAS-GTP complex. both of which have been proposed as targets for KRAS inhibition. However, due to low efficacy and target specificity of previously proposed therapeutic agents in directly inhibiting KRAS. current measures to target the KRAS pathway focus predominantly on inhibition of downstream effector proteins (Cox et al., Nat. Rev. Drug Discov. 13:828 (2014)). Nevertheless, despite challenges in developing a small molecule to directly down-regulate gene activity, KRAS remains a therapeutically relevant target due to, its prevalence as a driving mutation in human cancers. Recent work has revealed that c-Myc and mutant KRAS are tightly coupled (Vaseva et al. Cancer Cell, 34(5) (2018)), suggesting that the ability to co-silence both targets may have additive and/or synergistic anti-cancer effects.

The present invention overcomes the deficiencies in the art by providing compositions and methods using RNA interference for specific inhibition of c-Myc sequences and the combination of c-Myc and KRAS. Additionally, the present invention provides compositions and methods using a novel chimeric siRNA design, in which two chemically modified siRNAs are linked by a metabolically vulnerable phosphodiester DNA bridge, to specifically inhibit one or more target genes simultaneously, e.g., c-Myc and KRAS or any other genes of interest.

### SUMMARY OF THE INVENTION

The present invention is based on the development of inverted chimera siRNA molecules. These constructs provide remarkable inhibition of gene expression of one or more target genes and are unexpectly superior to single siRNAs and serial chimera siRNAs. Accordingly, one aspect of the invention relates to an inverted chimera siRNA molecule comprising a first and second siRNA in opposite orientation, the inverted chimera siRNA molecule comprising:
a) a first RNA comprising a first strand of the first siRNA and a first strand of the second siRNA connected by a linker:
b) a second RNA that is substantially complementary to the first strand of the first siRNA: and
c) a third RNA that is substantially complementary to the first strand of the second siRNA.

Another aspect of the invention relates to a composition, e.g., a pharmaceutical composition, comprising one or more of the inverted chimera siRNA molecules of the invention.

A further aspect of the invention relates to a method of inhibiting expression of a target gene in a cell, the method comprising contacting the cell with the inverted chimera siRNA molecule, the composition, and/or the pharmaceutical composition of the invention, thereby inhibiting expression of the target gene in the cell.

An additional aspect of the invention relates to a method of treating a disorder associated with a target gene in a subject in need thereof, the method comprising delivering to the subject the inverted chimera siRNA molecule, the composition, and/or the pharmaceutical composition of the invention, thereby treating the disorder in the subject.

Another aspect of the invention relates to a method of treating cancer in a subject in need thereof, wherein the cancer over-expresses a human c-Myc gene, the method comprising delivering to the subject the inverted chimera siRNA molecule of the invention, thereby treating cancer in the subject.

The present invention is further based on the identification of RNA molecules that inhibit expression of c-Myc sequences. Accordingly, one aspect of the invention relates to a double stranded RNA molecule comprising an antisense strand and a sense strand, wherein the nucleotide sequence of the antisense strand is complementary to a region of the nucleotide sequence of a human c-Myc gene, the region consisting essentially of about 18 to about 25 consecutive nucleotides: wherein the double stranded RNA molecule inhibits expression of a human c-Myc gene. Recent work has revealed that c-Myc and mutant KRAS are tightly coupled (Vaseva et al, Cancer Cell, 34(5) (2018), suggesting that the ability to co-silence both targets may have additive and/or synergistic anti-cancer effects. In one embodiment. multiple siRNAs are used to inhibit both c-Myc and KRAS genes simultaneously.

Another aspect of the invention relates to a composition, e.g., a pharmaceutical composition, comprising one or more of the RNA molecules of the invention.

A further aspect of the invention relates to a method of inhibiting expression of a human c-Myc gene the method comprising contacting the cell with the RNA molecule of the invention, thereby inhibiting expression of the human c-Myc gene in the cell.

An additional aspect of the invention relates to a method of treating cancer in a subject in need thereof, wherein the cancer comprises over-expression of a human c-Myc gene, the method comprising delivering to the subject the RNA molecule of the invention, thereby treating cancer in the subject.

Another aspect of the invention relates to the use of the RNA molecules of the invention to inhibit expression of a human c-Myc gene in a cell and to treat cancer in a subject in need thereof, wherein the cancer comprises over-expression of a human c-Myc gene.

Another aspect of the invention relates to a siRNA molecule targeted to a naturally-occurring human c-Myc mRNA, wherein the siRNA molecule comprises at least one chemical modification, and wherein the siRNA molecule comprises one of the following pairs of sequences:
sense strand of SEQ ID NO:37 and antisense strand of SEQ ID NO:38:
sense strand of SEQ ID NO:41 and antisense strand of SEQ ID NO:42:
sense strand of SEQ ID NO:43 and antisense strand of SEQ ID NO:44: or
sense strand of SEQ ID NO:45 and antisense strand of SEQ ID NO:46;
   or a sequence at least 90% identical thereto.

Another aspect of the invention relates to a composition, e.g., a pharmaceutical composition, comprising one or more of the siRNA molecules of the invention.

A further aspect of the invention relates to a method of inhibiting expression of a human c-Myc gene in a cell, the method comprising contacting the cell with the siRNAs molecules of the invention, thereby inhibiting expression of the human c-Myc gene in the cell.

An additional aspect of the invention relates to a method of treating cancer in a subject in need thereof, wherein the cancer comprises over-expression of a human c-Myc gene, the method comprising delivering to the subject the antisense oligonucleotides or siRNA molecules of the invention, thereby treating cancer in the subject.

Another aspect of the invention relates to the use of the siRNA molecules of the invention to inhibit expression of a human c-Myc gene in a cell and to treat cancer in a subject in need thereof, wherein the cancer comprises over-expression of a human c-Myc gene.

Another aspect of the invention relates to double stranded RNA molecules, a first RNA molecule comprising an antisense strand and a sense strand targeted to a human c-Myc gene and a second RNA molecule comprising an antisense strand and a sense strand targeted to a naturally-occuring KRAS sequence, which can encode a mutation selected from G12C, G12D, G12V, or G13D, wherein the nucleotide sequence of the first RNA molecule has an antisense strand that is complementary to a region of the nucleotide sequence of a human c-Myc gene, the region consisting essentially of about 18 to about 25 consecutive nucleotides: wherein the double stranded RNA molecule inhibits expression of a human c-Myc gene, and wherein the nucleotide sequence of the second RNA molecule has an antisense strand that is complementary to a region of the nucleotide sequence of a naturally-occuring KRAS sequence, which can encode a mutation selected from G12C, G12D, G12V, or G13D, the region consisting essentially of about 18 to about 25 consecutive nucleotides: wherein the double stranded RNA molecule inhibits expression of a naturally-occuring KRAS sequence encoding a mutation selected from G12C, G12D, G12V, G13D, or the KRAS wild-type sequence.

Another aspect of the invention relates to siRNA molecules, a first siRNA molecule targeted to a naturally-occurring human c-Myc mRNA, wherein the siRNA molecule comprises at least one chemical modification, and wherein the siRNA molecule comprises one of the following pairs of sequences:
sense strand of SEQ ID NO:37 and antisense strand of SEQ ID NO:38:
sense strand of SEQ ID NO:39 and antisense strand of SEQ ID NO:40:
sense strand of SEQ ID NO:41 and antisense strand of SEQ ID NO:42:
sense strand of SEQ ID NO:43 and antisense strand of SEQ ID NO:44: or
sense strand of SEQ ID NO:45 and antisense strand of SEQ ID NO:46;
or a sequence at least 90% identical thereto: and
   a second siRNA molecule targeted to a a naturally-occuring KRAS sequence that can encode a mutation selected from G12C, G12D, G12V, G13D, or the wild-type sequence wherein the siRNA molecule comprises at least one chemical modification. In some embodiments, the siRNA molecule comprises one of the following pairs of sequences:
sense strand of SEQ ID NO:47 and antisense strand of SEQ ID NO:48:
sense strand of SEQ ID NO:49 and antisense strand of SEQ ID NO:50:
   or a sequence at least 90% identical thereto.

Another aspect of the invention relates to a composition, e.g., a pharmaceutical composition, comprising one or more of the siRNA molecules of the invention.

A further aspect of the invention relates to a method of inhibiting expression of a human c-Myc gene and a naturally-occuring KRAS sequence that can encode a mutation selected from G12C, G12D, G12V, G13D, or the wild-type sequence in a cell, the method comprising contacting the cell with the siRNAs molecules of the invention, thereby inhibiting expression of the human c-Myc gene and a naturally-occuring KRAS sequence that can encode a mutation selected from G12C, G12D, G12V, G13D, or the wild-type sequence in the cell.

An additional aspect of the invention relates to a method of treating cancer in a subject in need thereof, wherein the cancer comprises over-expression of a human c-Myc gene and a naturally-occuring KRAS sequence that can encode a mutation selected from G12C, G12D, G12V, G13D, or the wild-type sequence the method comprising delivering to the subject siRNA molecules of the invention, thereby treating cancer in the subject.

Another aspect of the invention relates to the use of the siRNA molecules of the invention to inhibit expression of a human c-Myc gene and a naturally-occuring KRAS sequence that can encode a mutation selected from G12C, G12D, G12V, G13D, or the wild-type sequence in a cell and to treat cancer in a subject in need thereof, wherein the cancer comprises over-expression of a human c-Myc and a naturally-occuring KRAS sequence that can encode a mutation selected from G12C, G12D, G12V, G13D, or the wild-type sequence.

These and other aspects of the invention are set forth in more detail in the description of the invention below.

### BRIEF DESCRIPTION OF THE DRAWINGS

FIGS. 1A-IC show that synthetic siRNAs (“siRs”) can decrease c-Myc mRNA transcripts and protein expression in MIA-PaCa2 cells. A) MIA-PaCa2 cells were treated with a control siRNA targeting snord90) and eight c-Myc targeting siRNAs at 20 nM for 24 and 48 hours. B) MIA-PaCa2 cells were treated with a negative control siRNA targeting snord90, a positive control siRNA from previously published papers and eight c-Myc targeting siRNAs at 20 nM for 24 hours, then immunoblotted. Vinculin was used as an internal control. C) Quantification of c-Myc band intensity from Panel B.

FIGS. 2A-2B show that synthetic chemically modified siRNAs can substantially reduce c-Myc mRNA transcripts. A) MIA-PaCa2 cells were treated with a control siRNA with 2′Ome modifications and five chemically modified (Hi2F) c-Myc targeting siRNAs at 20 nM for 24, 48, and 72 hours. B) A427 cells were treated with a control siRNA with 2′Ome modifications and five chemically modified (Hi2F) c-Myc targeting siRNAs at 20 nM for 24 and 72 hours.

FIGS. 3A-3D show that synthetic chemically modified siRNAs can decrease c-Myc protein expression. A) MIA-PaCa2 and A427 cells were treated with a control siRNA with 2′Ome modifications and chemically modified (Hi2F) c-Myc targeting siRNAs at 20 nM for 48 (A427) and 72 (MIA-PaCa2) hours, then immunoblotted. Vinculin was used as an internal control. B) Quantification of c-Myc band intensity from Panel A. C) A427 cells were treated with a control siRNA with 2′Ome modifications and chemically modified (Hi2F and Hi2OMe) c-Myc targeting siRNAs at 20 nM for 48 hours. D) MIA-PaCa2 cells were treated with a control siRNA with 2′Ome modifications and chemically modified (Hi2F and Hi2OMe) c-Myc targeting siRNAs at 20 nM for 72 hours.

FIGS. 4A-4B show that synthetic chemically modified siRNAs decrease in-vitro spheroid formation. A) MIA-PaCa2 and A427 cells were treated with a control siRNA with 2′Ome modifications and two chemically modified (Hi2F) c-Myc targeting siRNAs at 20 nM for 24 hours, then grown in Matrigel for five days. Representative phase contrast images were taken with a confocal microscope using a 5× objective. B) Quantification of the average area of spheroids from the images in panel A. ****p=<0.0001, ***p=0.0002.

FIGS. 5A-5D show that dual-targeting c-Myc and KRAS with chemically modified siRNAs results in decreased spheroid area and number in MIA-PaCa2 cells. A) MIA-PaCa2 cells were treated with a control siRNA with 2′Ome modifications and two chemically modified c-Myc (Hi2F) and KRAS (Hi2OMe) targeting siRNAs at 5 nM for 24 hours, then grown in Matrigel for five days. Representative phase contrast images were taken with a confocal microscope using a 5× objective. B) Quantification of spheroid area and number from confocal images using Organoseg. *p=0.0027, **p=0.0147, ***p=0.0022, ****p=0.0113. C) MIA-PaCa2 cells were treated with a control siRNA with 2′Ome modifications and two chemically modified c-Myc (Hi2F) and KRAS (Hi2OMe) targeting siRNAs at 5 nM for 24 hours, then seeded in a 96-well round bottom plate and imaged for five days with the Incucyte. D) Post-imaging with the Incucyte, cells were mixed with CellTiter Glo 3D from Promega and fluorescence was measured with a plate reader to determine metabolic activity. ****p=<0.0001.

FIGS. 6A-6F show that dual-targeting c-Myc and KRAS with chemically modified siRNAs results in decreased spheroid area and number in A427 cells. A) A427 cells were treated with a control siRNA with 2′Ome modifications and two chemically modified c-Myc (Hi2F) and KRAS (Hi2OMe) targeting siRNAs at 10 nM for 24 hours, then grown in Matrigel for five days. Representative phase contrast images were taken with a confocal microscope using a 5× objective. B) Quantification of spheroid area and number from confocal images using Organoseg. ****p=<0.0001. C) A427 cells were treated with a control siRNA with 2′Ome modifications and two chemically modified c-Myc (Hi2F) and KRAS (Hi2OMe) targeting siRNAs at 10 nM for 24 hours, then seeded in a 96-well round bottom plate and imaged for five days with the Incucyte. D) Post-imaging with the Incucyte, cells were mixed with CellTiter Glo 3D from Promega and fluorescence was measured with a plate reader to determine metabolic activity. E,F) A427 cells were treated with a control siRNA with 2′Ome modifications and two chemically modified c-Myc (Hi2F) and KRAS (Hi2OMe) targeting siRNAs at 10 nM for 24 hours, then seeded in a 96-well round bottom plate with CellTox Green and imaged for five days with the Incucyte. Graphs show data up to 84 hours. **** p=<0.0001.

FIG. 7 is a schematic showing phosphodiester-linked chemically modified siRNA combining KRAS and cMyc-targeting siRs in two orientations (“inverted chimera” and “serial chimera”).

FIG. 8 shows that phosphodiester-linked chemically modified siR combining KRAS and cMyc-targeting siRs (“inverted chimera”) is biologically active and can decrease c-Myc and KRAS mRNA transcripts in MIA-PaCa2 cells. MIA-PaCa2 cells were treated with a control siRNA with 2′Ome modifications, one chemically modified (Hi2F) c-Myc-targeting siRNA and one chemically modified (Hi2OMe) KRAS-targeting siRNA (either alone or in combination), and one “inverted chimera” siRNA at 5 nM, 10 nM, and 20 nM for 48 and 72 hours.

FIG. 9 shows that inverted chimera is more potent at decreasing c-Myc and KRAS mRNA transcripts than serial chimera in A427 cells. A427 cells were treated with a control siRNA with 2′Ome modifications, one chemically modified (Hi2F) c-Myc-targeting siRNA, one chemically modified (Hi2OMe) KRAS-targeting siRNA, one M2/K2 “inverted chimera,” or one M2/K2 “serial chimera” siRNA at 5nM and 20nM for 48 and 72 hours.

FIGS. 10A-10C show that inverted chimera is more potent at decreasing c-Myc and KRAS mRNA transcripts than serial chimera in MIA-PaCa2 cells. A) MIA-PaCa2 cells were treated with a control siRNA with 2′Ome modifications, one chemically modified (Hi2F) c-Myc-targeting siRNA, one chemically modified (Hi2OMe) KRAS-targeting siRNA, one M2/K2 “inverted chimera.” or one M2/K2 “serial chimera” siRNA at 5 nM and 20 nM for 48 and 72 hours. B) MIA-PaCa2 cells were treated with a control siRNA with 2 Ome modifications, two chemically modified (Hi2F) c-Myc-targeting siRNAs, two chemically modified (Hi2OMe) c-Myc-targeting siRNAs, one chemically modified (Hi20Me) KRAS-targeting siRNA, one M2/K2 “inverted chimera.” or one M2/K2 “serial chimera” siRNA at 20 nM for 72 hours, then immunoblotted. Vinculin was used as an internal control. C) Quantification of c-Myc and KRAS band intensities from B.

FIG. 11 shows that inverted chimera is more stable in 50% serum than serial chimera. One chemically modified (Hi2F) c-Myc-targeting siRNA, one chemically modified (Hi2OMe) KRAS-targeting siRNA, one M2/K2 “inverted chimera,” or one M2/K2 “serial chimera” siRNA at 10 uM were incubated in 50% FBS at 37° C. for 0), 6, and 24 hours. Reactions were stopped with 0.5M EDTA. Quantification of siR band intensity is shown on the right.

FIG. 12 shows that both chimeras are stable in cytosolic conditions. One chemically modified (Hi2F) c-Myc-targeting siRNA, one chemically modified (Hi2OMe) KRAS-targeting siRNA, one M2/K2 “inverted chimera,” or one M2/K2 “serial chimera” siRNA were incubated in buffered rat liver cytosol at 37° C. for 0, 6, and 24 hours. Reactions were stopped with 0.5M EDTA. Quantification of siR band intensity is shown on the right.

FIG. 13 shows both chimeras begin to dissociate in endosomal conditions. One chemically modified (Hi2F) c-Myc-targeting siRNA, one chemically modified (Hi2OMe) KRAS-targeting siRNA, one M2/K2 “inverted chimera,” or one M2/K2 “serial chimera” siRNA were incubated in acidified rat liver tritosomes at 37° C. for 0, 6, and 24 hours. Reactions were stopped with 0.5M EDTA. Quantification of siR band intensity is shown on the right.

FIGS. 14A-14C show that dicer does not cleave either the inverted or the serial chimera. A) One chemically modified (Hi2F) c-Myc-targeting siRNA, one chemically modified (Hi2OMe) KRAS-targeting siRNA, one M2/K2 “inverted chimera,” or one M2/K2 “serial chimera” siRNA were incubated with buffered recombinant Dicer at 37° C. for 0, 6, and 24 hours. Reactions were stopped with 0.5M EDTA. Quantification of siR band intensity is shown on the right. HEK293T Parental (B) and NoDice (C) cell lines were treated with a control siRNA with 2′Ome modifications, one chemically modified (Hi2F) c-Myc-targeting siRNA, one chemically modified (Hi20Me) KRAS-targeting siRNA, one M2/K2 “inverted chimera.” or one M2/K2 “serial chimera” siRNA at 5 nM and 20 nM for 48 hours.

FIG. 15 shows that inverted chimera results in increased potency against KRAS-luciferase protein than the serial chimera. An A431 isogenic cell line that has endogenous KRAS removed by CRISPR/Cas9, and stably integrates KRAS-Firefly luciferase and untagged Renilla luciferase was treated with a control siRNA with 2′Ome modifications, one chemically modified (Hi2F) c-Myc-targeting siRNA, one chemically modified (Hi2OMe) KRAS-targeting siRNA, one M2/K2 “inverted chimera,” or one M2/K2 “serial chimera” siRNA for 96 hours at doses ranging from 39 to 0.002 nM. Firefly luciferase data was normalized with Renilla luciferase.

FIGS. 16A-16B show that V2 of inverted chimera results in equivalent potency against c-Myc and KRAS mRNA as V1. MIA-PaCa2 (A) and A427 (B) cells were treated with a control siRNA with 2′Ome modifications, one M2/K2 “inverted chimera” version 1 siRNA, or one M2/K2 “inverted chimera” version 2 siRNA for 72 hours at 4 nM and 10 nM.

FIGS. 17A-17B show that V2 of inverted chimera results in increased reduction of c-Myc and KRAS protein than single siRs in A427 cells. A) A427 cells were treated with a control siRNA with 2′Ome modifications, one chemically modified (Hi2OMe) c-Myc-targeting siRNA, one chemically modified (Hi2OMe) KRAS-targeting siRNA, or one M2/K2 “inverted chimera” version 2 siRNA at 5 nM and 20 nM for 72 hours, then immunoblotted. Vinculin was used as an internal control. B) Quantification of c-Myc, KRAS, pERK(1/2)/tERK(1/2), and pS6 band intensity from Panel A. All conditions were normalized with vinculin band intensity.

FIGS. 18A-18B show that V2 of inverted chimera results in increased reduction of c-Myc and KRAS protein than single siRs in MIA-PaCa2 cells. A) MIA-PaCa2 cells were treated with a control siRNA with 2′Ome modifications, one chemically modified (Hi2OMe) c-Myc-targeting siRNA, one chemically modified (Hi2OMe) KRAS-targeting siRNA, or one M2/K2 “inverted chimera” version 2 siRNA at 5 nM and 20 nM for 72 hours, then immunoblotted. Vinculin was used as an internal control. B) Quantification of c-Myc, KRAS, and pERK(1/2)/tERK(1/2) band intensity from A. All conditions were normalized with vinculin band intensity.

FIG. 19 shows that V2 of inverted chimera results in high potency against KRAS-luciferase protein. A431 KRAS-knockout cell line with stably integrated KRAS-Firefly luciferase and untagged Renilla luciferase was treated with a control siRNA with 2′Ome modifications, one chemically modified (Hi2OMe) c-Myc-targeting siRNA, one chemically modified (Hi2OMe) KRAS-targeting siRNA, or one M2/K2 “inverted chimera” version 2 siRNA for 96 hours at doses ranging from 21 to 0.001 nM. Firefly luciferase data was normalized with Renilla luciferase.

FIGS. 20A-20B show that inverted chimera V2 synergistically decreases cell viability in multiple cell lines. MIA-PaCa2 (A) and A427 (B) cells were treated with a control siRNA with 2′Ome modifications, one chemically modified (Hi2OMe) c-Myc-targeting siRNA, one chemically modified (Hi2Me) KRAS-targeting siRNA, or one M2/K2 “inverted chimera” version 2 siRNA for 120 hours at doses ranging from 40 nM to 0.019 nM.

FIGS. 21A-21D show inverted chimera V2 decreases spheroid viability in multiple cell lines. A427 (A) and MIA-PaCa2 (B) cells were treated with a control siRNA with 2′Ome modifications, one chemically modified (Hi2OMe) c-Myc-targeting siRNA, one chemically modified (Hi2OMe) KRAS-targeting siRNA, or one M2/K2 “inverted chimera” version 2 siRNA at 5nM for 24 hours, then grown in Matrigel for five days. Representative phase contrast images were taken with a confocal microscope using a 5× objective. C) Quantification of spheroid area and number in A427 from confocal images using Organoseg. ***p=0.004, **p=0.0084, ****p=<0.001. D) Quantification of spheroid area and number in A427. *p=0.0435, **p=0.0182, ***p=0.042, ****p=0.0012.

FIG. 22 shows that development and validation of specific KRAS mutation (G12V) siRNAs using isogenic cell lines expressing KRAS WT or KRAS G12V reporters. A431 cells had the endogenous KRAS WT allele deleted with CRISPR. Then, a stable KRAS WT or KRAS G12V expression vector was expressed. On an mRNA level, using RT-qPCR, 12V FM4) resulted in potent silencing of KRAS G12V but fully spared KRAS WT targeting. Compared to the pan-KRAS siRNA. using a Luciferase readout. it was found that EFTX-G12V-FM4 potently targets KRAS G12V but fully spares targeting of KRAS WT.

FIG. 23 shows that development and validation of specific KRAS mutation (G12V) siRNAs using SKCO1 (colon) and H727 (lung) cell lines with KRAS G12V mutations. SKCO1 and H727 cell lines (with G12V mutation) were treated with a control siRNA with 2′Ome modifications, one chemically modified (Hi2OMe) KRAS-targeting siRNA. or one EFTX-G12V siRNAs (Hi2OMe) G12V mutant-specific KRAS-targeting siRNA for about one week at doses ranging from 40 nM to 0.01 nM.

FIG. 24 shows that inverted chimera targeting a specific KRAS mutation (G12V) and c-Myc synergistically decreases cell viability in H441. H441 (with G12V mutation) cell line was treated with a control siRNA with 2 Ome modifications. one chemically modified (Hi2OMe) KRAS-targeting siRNA. one chemically modified (Hi2OMe) G12V mutant-specific KRAS-targeting siRNA, one M2/K2 inverted chimera version 2 siRNA. or one M2/G12V-specific inverted chimera for 144 hours at doses ranging from 40 nM to 0.01 nM.

FIG. 25 shows that inverted chimera targeting a specific KRAS mutation (G12V) and c-Myc synergistically decreases cell viability in SKCO1. SKCO1 (with G12V mutation) cell line was treated with a control siRNA with 2′Ome modifications. one chemically modified (Hi2OMe) KRAS-targeting siRNA, one chemically modified (Hi2OMe) G12V mutant-specific KRAS-targeting siRNA, one M2/K2 inverted chimera version 2 siRNA. or one M2/G12V-specific inverted chimera for 216 hours at doses ranging from 40 nM to 0.01 nM.

FIG. 26 shows that inverted chimera targeting an IRF4 and c-Myc synergistically decreases cell viability in RPMI-8226 myeloma cell lines. RPMI-8226 cells were treated with varying doses of HiOMe siRNAs complexed with RNAiMax in 96-well plate format for 5 days, with 2OMe as the negative control. On the 5th day post-transfection, Cell Titer Glo 2.0 was added and luminescence was determined.

FIGS. 27A-27B show that inverted chimera targeting the same gene (c-Myc) is more effective at mRNA reduction than targeting the gene with individual siRs. A427 (A) and MIA-PaCa2 (B) cells were treated with a control siRNA with 2′Ome modifications. two chemically modified (Hi2OMe) c-Myc-targeting siRNA, one M2/M3 inverted chimera siRNA. or one M3/M2 inverted chimera siRNA at 5nM for 72 hours.

FIG. 28 shows that inverted chimera targeting the same gene (KRAS) is more effective at reducing KRAS-luciferase protein than targeting the gene with individual siRs. A431 KRAS-knockout cell line with stably integrated KRAS-Firefly luciferase and untagged Renilla luciferase was treated with a control siRNA with 2′Ome modifications, one chemically modified (Hi20Me) c-Myc-targeting siRNA, one chemically modified (Hi2OMe) KRAS-targeting siRNA, or one K3/K2 inverted chimera siRNA for 96 hours at doses ranging from 39 to 0.002 nM. Firefly luciferase data was normalized with Renilla luciferase.

FIG. 29 shows potential conjugation sites (black arrows) for linkers and targeting moieties for both the serial and inverted chimeras.

FIG. 30 is a schematic showing phosphodiester-linked chemically modified siR combining up to 3 unique siRNAs using a combined inverted chimera and serial chimera design.

### DETAILED DESCRIPTION OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. §1.822 and established usage.

Except as otherwise indicated, standard methods known to those skilled in the art may be used for cloning genes, amplifying and detecting nucleic acids, and the like. Such techniques are known to those skilled in the art. See. e.g., Green et al., Molecular Cloning: A Laboratory Manual 4th Ed. (Cold Spring Harbor, NY, 2012): Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

### Definitions

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a

polynucleotide sequence of this invention, means a polynucleotide that consists of both the recited sequence (e.g., SEQ ID NO) and a total of ten or less (e.g., 1, 2, 3, 4, 5, 6, 7, 8, 9, or 10) additional nucleotides on the 5′ and/or 3′ ends of the recited sequence such that the function of the polynucleotide is not materially altered. The total of ten or less additional nucleotides includes the total number of additional nucleotides on both ends added together. The term “materially altered,” as applied to polynucleotides of the invention, refers to an increase or decrease in ability to inhibit expression of a target mRNA of at least about 50% or more as compared to the expression level of a polynucleotide consisting of the recited sequence.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject (e.g., in the case of cancer, reduction in tumor burden, prevention of further tumor growth, prevention of metastasis, or increase in survival time). Those skilled in the art will appreciate that the therapeutic effects need not be complete or curative, as long as some benefit is provided to the subject.

By the terms “treat,” “treating,” or “treatment of,” it is intended that the severity of the subject's condition is reduced or at least partially improved or modified and that some alleviation, mitigation or decrease in at least one clinical symptom is achieved.

“Prevent” or “preventing” or “prevention” refer to prevention or delay of the onset of the disorder and/or a decrease in the severity of the disorder in a subject relative to the severity that would develop in the absence of the methods of the invention. The prevention can be complete, e.g., the total absence of cancer in a subject. The prevention can also be partial, such that the occurrence or severity of cancer in a subject is less than that which would have occurred without the present invention.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

An “isolated polynucleotide” is a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. Thus, in one embodiment, an isolated nucleic acid includes some or all of the 5′ non-coding (e.g., promoter) sequences that are immediately contiguous to a coding sequence. The term therefore includes, for example, a recombinant DNA that is incorporated into a vector, into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment), independent of other sequences. It also includes a recombinant DNA that is part of a hybrid nucleic acid encoding an additional polypeptide or peptide sequence. An isolated polynucleotide that includes a gene is not a fragment of a chromosome that includes such gene, but rather includes the coding region and regulatory regions associated with the gene, but no additional genes naturally found on the chromosome.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

The term “fragment.” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., two, three, four, five or more heterologous nucleotide sequences.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, and/or adenovirus vectors. Non-viral vectors include, but are not limited to, plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

Vectors may be introduced into the desired cells by methods known in the art, e.g., transfection, electroporation, microinjection, transduction, cell fusion, DEAE dextran, calcium phosphate precipitation, lipofection (lysosome fusion), use of a gene gun, or a nucleic acid vector transporter (see, e.g., Wu et al., J. Biol. Chem. 267:963 (1992): Wu et al., J. Biol. Chem. 263:14621 (1988); and Hartmut et al., Canadian Patent Application No. 2,012,311, filed Mar. 15, 1990).

In some embodiments, a polynucleotide of this invention can be delivered to a cell in vivo by lipofection. Synthetic cationic lipids designed to limit the difficulties and dangers encountered with liposome-mediated transfection can be used to prepare liposomes for in vivo transfection of a nucleotide sequence of this invention (Felgner et al., Proc. Natl. Acad. Sci. USA 84:7413 (1987): Mackey, et al., Proc. Natl. Acad. Sci. U.S.A. 85:8027 (1988); and Ulmer et al., Science 259:1745 (1993)). The use of cationic lipids may promote encapsulation of negatively charged nucleic acids, and also promote fusion with negatively charged cell membranes (Felgner et al., Science 337:387 (1989)). Particularly useful lipid compounds and compositions for transfer of nucleic acids are described in International Patent Publications WO95/18863 and WO96/17823, and in U.S. Pat. No. 5,459,127. The use of lipofection to introduce exogenous nucleotide sequences into specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting (Mackey, et al., 1988, supra). Targeted peptides, e.g., hormones or neurotransmitters, and proteins such as antibodies, or non-peptide molecules can be coupled to liposomes chemically.

In various embodiments, other molecules can be used for facilitating delivery of a nucleic acid in vivo, such as a cationic oligopeptide (e.g., WO95/21931), peptides derived from nucleic acid binding proteins (e.g., WO96/25508), and/or a cationic polymer (e.g., WO95/21931).

It is also possible to introduce a vector in vivo as naked nucleic acid (see U.S. Pat. Nos. 5,693,622, 5,589,466 and 5,580,859). Receptor-mediated nucleic acid delivery approaches can also be used (Curiel et al., Hum. Gene Ther. 3:147 (1992); Wu et al., J. Biol. Chem. 262:4429 (1987)).

As used herein, the terms “protein” and “polypeptide” are used interchangeably and encompass both peptides and proteins, unless indicated otherwise.

A “fusion protein” is a polypeptide produced when two heterologous nucleotide sequences or fragments thereof coding for two (or more) different polypeptides not found fused together in nature are fused together in the correct translational reading frame. Illustrative fusion polypeptides include fusions of a polypeptide of the invention (or a fragment thereof) to all or a portion of glutathione-S-transferase, maltose-binding protein, or a reporter protein (e.g., Green Fluorescent Protein, β-glucuronidase, β-galactosidase, luciferase, etc.), hemagglutinin, c-Myc, FLAG epitope, etc.

By the term “express” or “expression” of a polynucleotide coding sequence, it is meant that the sequence is transcribed, and optionally, translated. Typically, according to the present invention, expression of a coding sequence of the invention will result in production of the polypeptide of the invention. The entire expressed polypeptide or fragment can also function in intact cells without purification.

As used herein, the term “over-expression” or “over-expressing” refers to increased levels of a polypeptide being produced and/or increased time of expression (e.g., constitutively expressed) compared to a wild-type cell.

As used herein, the term “gene” refers to a nucleic acid molecule capable of being used to produce mRNA, antisense RNA, miRNA, and the like. Genes may or may not be capable of being used to produce a functional protein. Genes can include both coding and non-coding regions (e.g., introns, regulatory elements, promoters, enhancers, termination sequences and 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

The terms “complementary” or “complementarity,” as used herein, refer to the natural binding of polynucleotides under permissive salt and temperature conditions by base-pairing. Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

As used herein, the terms “substantially complementary.” or “partially complementary” mean that two nucleic acid sequences are complementary at least about 60%, 70%, 80% or 90% of their nucleotides. In some embodiments, the two nucleic acid sequences can be complementary at least at 85%, 90%, 95%, 96%, 97%, 98%, 99% or more of their nucleotides. The terms “substantially complementary” and “partially complementary” can also mean that two nucleic acid sequences can hybridize under high stringency conditions and such conditions are well known in the art.

As used herein, “heterologous” refers to a nucleic acid sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., a different copy number, and/or under the control of different regulatory sequences than that found in nature.

As used herein, the terms “contacting,” “introducing” and “administering” are used interchangeably, and refer to a process by which dsRNA of the present invention or a nucleic acid molecule encoding a dsRNA of this invention is delivered to a cell, in order to inhibit or alter or modify expression of a target gene. The dsRNA may be administered in a number of ways, including, but not limited to, direct introduction into a cell (i.e., intracellularly) and/or extracellular introduction into a cavity, interstitial space, or into the circulation of the organism.

“Introducing” in the context of a cell or organism means presenting the nucleic acid molecule to the organism and/or cell in such a manner that the nucleic acid molecule gains access to the interior of a cell. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into cells in a single transformation event or in separate transformation events. Thus, the term “transformation” as used herein refers to the introduction of a heterologous nucleic acid into a cell. Transformation of a cell may be stable or transient.

“Transient transformation” in the context of a polynucleotide means that a polynucleotide is introduced into the cell and does not integrate into the genome of the cell.

By “stably introducing” or “stably introduced” in the context of a polynucleotide introduced into a cell, it is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and mitochondrial genome, and therefore includes integration of the nucleic acid into, for example, the mitochondrial genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism. Stable transformation of a cell can be detected by, for example, a Northern blot hybridization assay of RNA of the cell with nucleic acid sequences which specifically hybridize with a nucleotide sequence of a transgene introduced into an organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Embodiments of the invention are directed to expression cassettes designed to express the nucleic acids of the present invention. As used herein, “expression cassette” means a nucleic acid molecule having at least a control sequence operably linked to a nucleotide sequence of interest. In this manner, for example, promoters in operable interaction with the nucleotide sequences for the siRNAs of the invention are provided in expression cassettes for expression in an organism or cell.

As used herein, the term “promoter” refers to a region of a nucleotide sequence that incorporates the necessary signals for the efficient expression of a coding sequence. This may include sequences to which an RNA polymerase binds, but is not limited to such sequences and can include regions to which other regulatory proteins bind together with regions involved in the control of protein translation and can also include coding sequences.

Furthermore, a “promoter” of this invention is a promoter capable of initiating transcription in a cell of an organism. Such promoters include those that drive expression of a nucleotide sequence constitutively, those that drive expression when induced, and those that drive expression in a tissue-or developmentally-specific manner, as these various types of promoters are known in the art.

For purposes of the invention, the regulatory regions (i.e., promoters, transcriptional regulatory regions, and translational termination regions) can be native/analogous to the organism or cell and/or the regulatory regions can be native/analogous to the other regulatory regions. Alternatively, the regulatory regions may be heterologous to the organism or cell and/or to each other (i.e., the regulatory regions). Thus, for example, a promoter can be heterologous when it is operably linked to a polynucleotide from a species different from the species from which the polynucleotide was derived. Alternatively, a promoter can also be heterologous to a selected nucleotide sequence if the promoter is from the same/analogous species from which the polynucleotide is derived, but one or both (i.e., promoter and polynucleotide) are substantially modified from their original form and/or genomic locus, or the promoter is not the native promoter for the operably linked polynucleotide.

The choice of promoters to be used depends upon several factors, including, but not limited to, cell-or tissue-specific expression, desired expression level, efficiency, inducibility and selectability. For example, where expression in a specific tissue or organ is desired, a tissue-specific promoter can be used. In contrast, where expression in response to a stimulus is desired, an inducible promoter can be used. Where continuous expression is desired throughout the cells of an organism, a constitutive promoter can be used. It is a routine matter for one of skill in the art to modulate the expression of a nucleotide sequence by appropriately selecting and positioning promoters and other regulatory regions relative to that sequence.

In addition to the promoters described above, the expression cassette also can include other regulatory sequences. As used herein, “regulatory sequences” means nucleotide sequences located upstream (5′ non-coding sequences), within or downstream (3′ non-coding sequences) of a coding sequence, and which influence the transcription, RNA processing or stability, or translation of the associated coding sequence. Regulatory sequences include, but are not limited to, enhancers, introns, translation leader sequences and polyadenylation signal sequences.

The expression cassette also can optionally include a transcriptional and/or translational termination region (i.e., termination region) that is functional in the organism. A variety of transcriptional terminators are available for use in expression cassettes and are responsible for the termination of transcription beyond the transgene and correct mRNA polyadenylation. The termination region may be native to the transcriptional initiation region, may be native to the operably linked nucleotide sequence of interest, may be native to the host, or may be derived from another source (i.e., foreign or heterologous to the promoter, the nucleotide sequence of interest, the host, or any combination thereof).

A signal sequence can be operably linked to nucleic acids of the present invention to direct the nucleotide sequence into a cellular compartment. In this manner, the expression cassette will comprise a nucleotide sequence encoding the siRNA operably linked to a nucleic acid sequence for the signal sequence. The signal sequence may be operably linked at the N-or C-terminus of the siRNA.

Regardless of the type of regulatory sequence(s) used, they can be operably linked to the nucleotide sequence of the siRNA. As used herein, “operably linked” means that elements of a nucleic acid construct such as an expression cassette are configured so as to perform their usual function. Thus, regulatory or control sequences (e.g., promoters) operably linked to a nucleotide sequence of interest are capable of effecting expression of the nucleotide sequence of interest. The control sequences need not be contiguous with the nucleotide sequence of interest, so long as they function to direct the expression thereof. Thus, for example, intervening untranslated, yet transcribed, sequences can be present between a promoter and a coding sequence, and the promoter sequence can still be considered “operably linked” to the coding sequence. A nucleotide sequence of the present invention (i.e., a siRNA) can be operably linked to a regulatory sequence, thereby allowing its expression in a cell and/or subject.

The expression cassette also can include a nucleotide sequence for a selectable marker, which can be used to select a transformed organism or cell. As used herein, “selectable marker” means a nucleic acid that when expressed imparts a distinct phenotype to the organism or cell expressing the marker and thus allows such transformed organisms or cells to be distinguished from those that do not have the marker. Such a nucleic acid may encode either a selectable or screenable marker, depending on whether the marker confers a trait that can be selected for by chemical means, such as by using a selective agent (e.g., an antibiotic or the like), or on whether the marker is simply a trait that one can identify through observation or testing, such as by screening (. Of course, many examples of suitable selectable markers are known in the art and can be used in the expression cassettes described herein.

In some embodiments of the present invention, the expression cassette can comprise an expression control sequence operatively linked to a nucleotide sequence that is a template for one or both strands of the dsRNA. In further embodiments, a promoter can flank either end of the template nucleotide sequence, wherein the promoters drive expression of each individual DNA strand, thereby generating two complementary (or substantially complementary) RNAs that hybridize and form the dsRNA. In alternative embodiments, the nucleotide sequence is transcribed into both strands of the dsRNA on one transcription unit, wherein the sense strand is transcribed from the 5′ end of the transcription unit and the antisense strand is transcribed from the 3′ end, wherein the two strands are separated by about 3 to about 500 basepairs, and wherein after transcription, the RNA transcript folds on itself to form a short hairpin RNA (shRNA) molecule.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993): Computer Analysis of Sequence Data, Part I (Griffin, A. M., and Griffin, H. G., eds.) Humana Press, New Jersey (1994): Sequence Analysis in Molecular Biology (von Heinje, G., ed.) Academic Press (1987): and Sequence Analysis Primer (Gribskov, M. and Devereux, J., eds.) Stockton Press, New York (1991).

As used herein, the term “substantially identical” or “corresponding to” means that two nucleic acid sequences have at least 60%, 70%, 80% or 90% sequence identity. In some embodiments, the two nucleic acid sequences can have at least 85%, 90%, 95%, 96%, 97%, 98%, 99% or 100% of sequence identity.

An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCGR Wisconsin Package R (Accelrys Inc., Burlington, Mass.). Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0) for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo, H., and Lipton, D., (Applied Math 48:1073(1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894: see BLAST Manual, Altschul et al., NCBI, NLM, NIH: (Altschul et al., J. Mol. Biol. 215:403-410 (1990)): version 2.0 or higher of BLAST programs allows the introduction of gaps (deletions and insertions) into alignments: for peptide sequence BLASTX can be used to determine sequence identity: and, for polynucleotide sequence BLASTN can be used to determine sequence identity.

As used herein, “RNAi” or “RNA interference” refers to the process of sequence-specific post-transcriptional gene silencing, mediated by double-stranded RNA (dsRNA). As used herein, “dsRNA” refers to RNA that is partially or completely double stranded. Double stranded RNA is also referred to as small interfering RNA (siRNA), small interfering nucleic acid (siNA), microRNA (miRNA), and the like. In the RNAi process, dsRNA comprising a first (antisense) strand that is complementary to a portion of a target gene and a second (sense) strand that is fully or partially complementary to the first antisense strand is introduced into an organism. After introduction into the organism, the target gene-specific dsRNA is processed into relatively small fragments (siRNAs) and can subsequently become distributed throughout the organism, leading to a phenotype that, over the period of a generation, may come to closely resemble the phenotype arising from a complete or partial deletion of the target gene.

MicroRNAs (miRNAs) are non-protein coding RNAs, generally of between about 18 to about 25 nucleotides in length. These miRNAs direct cleavage in trans of target transcripts, negatively regulating the expression of genes involved in various regulation and development pathways (Bartel, Cell, 116:281-297 (2004): Zhang et al. Dev. Biol. 289:3-16 (2006)). As such, miRNAs have been shown to be involved in different aspects of growth and development as well as in signal transduction and protein degradation. Since the first miRNAs were discovered in plants (Reinhart et al. Genes Dev. 16:1616-1626 (2002), Park et al. Curr. Biol. 12:1484-1495 (2002)) many hundreds have been identified. Many microRNA genes (MIR genes) have been identified and made publicly available in a database (miRBase; microma.sanger.ac.uk/sequences). miRNAs are also described in U.S. Patent Publications 2005/0120415 and 2005/144669A1, the entire contents of which are incorporated by reference herein.

Genes encoding miRNAs yield primary miRNAs (termed a “pri-miRNA”) of 70 to 300 bp in length that can form imperfect stem-loop structures. A single pri-miRNA may contain from one to several miRNA precursors. In animals, pri-miRNAs are processed in the nucleus into shorter hairpin RNAs of about 65 nt (pre-miRNAs) by the RNaseIII enzyme Drosha and its cofactor DGCR8/Pasha. The pre-miRNA is then exported to the cytoplasm, where it is further processed by another RNaseIII enzyme, Dicer, releasing a miRNA/miRNA* duplex of about 22 nt in size. Many reviews on microRNA biogenesis and function are available, for example, see. Bartel Cell 116:281-297 (2004), Murchison et al. Curr. Opin. Cell Biol. 16:223-229 (2004), Dugas et al. Curr. Opin. Plant Biol. 7:512-520 (2004) and Kim Nature Rev. Mol. Cell Biol. 6:376-385 (2005).

### RNA Molecules

The invention relates to a RNA molecule that is a multivalent inverted chimeric double stranded RNA or chemically-modified siRNA molecule. The inverted chimera is surprisingly effective at inhibiting gene expression and is unexpectedly far more potent against both target genes relative to individual siRNAs or chimeric siRNAs placed in a serial format. The inverted chimeric molecule advantageously simplifies the siRNA delivery process and ensures equivalent molar targeting of both gene transcripts. The chimeric molecule design also simplifies synthesis of two siRNAs because the linker is a short nucleotide bridge that is demonstrated to be cleavable within the endosome.

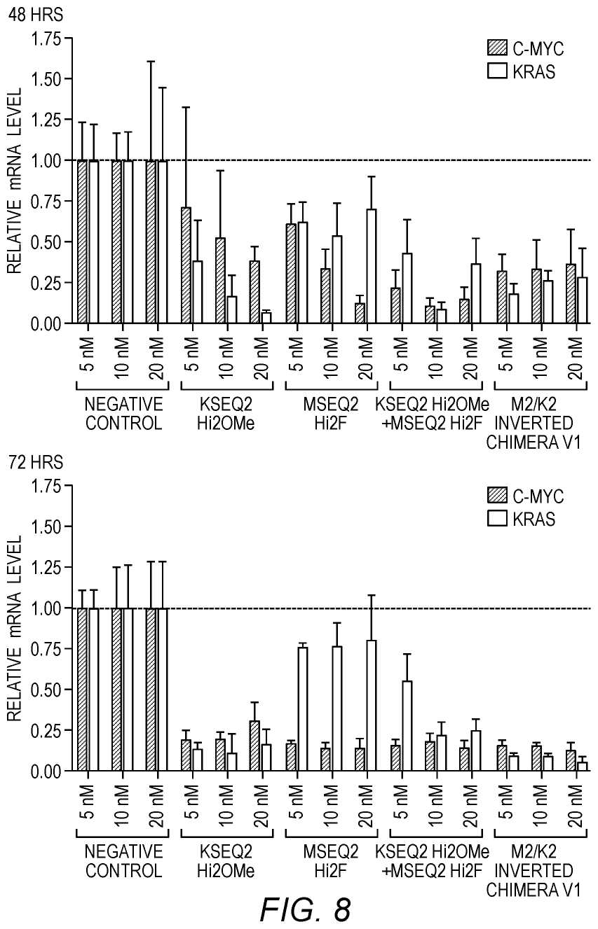

An inverted chimera, as used herein, is a molecule comprising two siRNAs in tandem to each other but inverted with regard to sense and antisense orientation (i.e., the first strand is a sense (passenger) strand in a 5′ to 3′ orientation, followed by a linker (e.g., phosphodiester DNA bridge), and the second strand is an antisense (guide) strand also in a 5° to 3′ orientation). A serial chimera is also in tandem but has the antisense strands of the siRNAs in the same 5° to 3′ orientation. The chimera is composed of three RNA molecules. The first RNA molecule comprises a first strand of each siRNA, optionally connected by a linker. In an inverted chimera, the first RNA molecule comprises the sense strand of one siRNA and the antisense strand of the second siRNA. In a serial chimera, the first RNA molecule comprises the antisense strand of one siRNA and the antisense strand of the second siRNA. The second RNA molecule is substantially complementary to the the first strand of the first siRNA and the third RNA molecule is substantially complementary to the first strand of the second siRNA. See, for example, FIG. 7.

The inverted chimeric molecule may have an overall length of about 30 to 60 nucleotides, e.g., about 35 to 55 nucleotides, e.g., about 40 to 50 nucleotides. Each of the siRNAs within the inverted chimeric molecule may have a length of about 15 to 30 nucleotides, e.g., about 17 to 25 nucleotides, e.g., about 19 to 23 nucleotides.

The two siRNAs may be connected by a linker, e.g., a metabolically-vulnerable linker (i.e., a linker that is cleaved when the inverted chimeric molecule is delivered to a cell or administered to a subject), e.g., a polynucleotide linker. The linker may be about 2-10 nucleotides in length, e.g., about 3-8 nucleotides, e.g., 4-6 nucleotides. The nucleotides in the linker may be DNA or RNA, e.g., phosphodiester thymines, phosphodiester adenines, a phosphodiester thymine/cytosine/adenine (TCA) linker, etc.

One aspect of the invention relates to an inverted chimeric siRNA molecule of the invention, wherein the molecule comprises as least one chemical modification. In some embodiments, the molecule is fully chemically modified. The term “fully chemically-modified” means that every nucleotide in the molecule contains a chemical modification. In some embodiments, each nucleotide in the molecule is modified with a 2′-O-methyl group or a 2′-fluoro group.

In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 of the nucleotide linkages in the inverted chimeric siRNA molecule are chemically modified. In some embodiments, the inverted chimeric siRNA molecule comprises at least one phosphorothioate linkage. In some embodiments, the siRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 phosphorothioate linkages. In some embodiments, a phosphorothioate linkage is placed between the last 2 nucleotides of each strand of the molecule. In some embodiments, the molecule comprises all phosphorothioate linkages.

Each siRNA in the inverted chimeric molecule is capable of binding to a mRNA of a target gene and inhibiting expression of that gene. In some embodiments, each siRNA in the inverted chimeric molecule binds to the same target gene, e.g., to different portions of the gene. In other embodiments, each siRNA in the inverted chimeric molecule binds to a different target gene, e.g., to efficiently inhibit expression of two genes simultaneously.

The target gene may be any gene of interest for which inhibition of expression is desired. In some embodiments, the target gene is one being studied for research purposes. In other embodiments, the target gene is one that is over-expressed in a disease state or otherwise associated with a disease state, and inhibition of expression is for therapeutic or prophylactic purposes. In some embodiments, the target gene is one that is expressed by a pathogen, e.g., a bacteria, virus, fungus, or parasite, e.g., a coronavirus such as SARS-CoV-2. In some embodiments, the target gene is one over-expressed in or associated with a cancer. In a particular embodiment, at least one siRNA targets c-Myc. In one embodiment, one siRNA targets c-Myc and one siRNA targets KRAS.

Each siRNA in the inverted chimeric molecule may be a known siRNA or one later developed. In some embodiments at least one of the siRNAs is an siRNA disclosed herein.

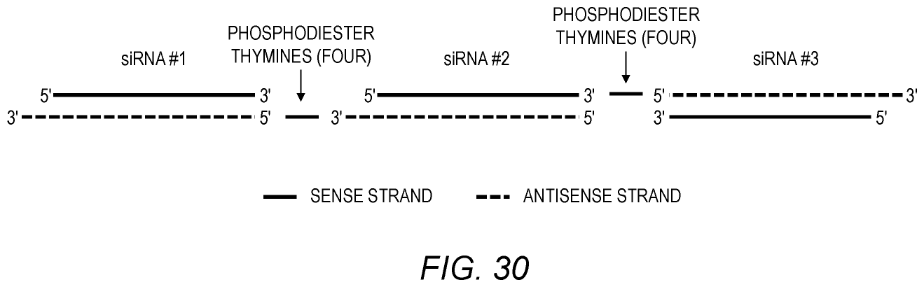

In some embodiments, the inverted chimeric molecule may be duplexed with additional serial siRNAs attached thereto, e.g., 2, 3, 4, 5, 6, 7, 8, 9, 10, or more. See, e.g., FIG. 30, which demonstrates an inverted chimeric design duplexed with a serial chimera, which results in a total of 3 possible unique siRNA sequences. Each siRNA may be separated by a linker as described above. Each siRNA in the inverted chimeric molecule may independently bind to and inhibit the same target gene or a different target gene. Each additional siRNA may be in serial or inverted orientation relative to the siRNAs in the inverted chimeric molecule. Each pair of additional siRNAs may be in an inverted chimeric format, e.g., a chain of inverted chimera molecules.

In some embodiments, the inverted chimera molecule may be covalenty or non-covalently bound to another molecule, e.g., a linker (e.g., for binding to a carrier) or a ligand (e.g., for targeting the molecule to a specific cell or binding site). Suitable sites for conjugation are shown in FIG. 29.

In some embodiments, the chimera molecule comprises, consists essentially of, or consists of a set of sequences at least 90% identical to one of:
SEQ ID NOS:63-65
SEQ ID NOS:66-68
SEQ ID NOS:69-71
SEQ ID NOS:71-74
SEQ ID NOS:75-77
SEQ ID NOS:78-80
SEQ ID NOS:81-83
SEQ ID NOS:84-86
SEQ ID NOS:87-89
SEQ ID NOS:90-92
SEQ ID NOS:93-95
SEQ ID NOS:96-98
SEQ ID NOS:99-101
SEQ ID NOS:102-104
   e.g., at least 90%, 91%, 92%, 93%, 94%, 95%, 96%, 97%, 98%, or 99% identical.

A second aspect of the invention presents an alternative therapeutic approach by targeting c-Myc at the transcriptional level. The invention consists of short interfering RNAs (siRNAs) that can complementarily bind to c-Myc messenger RNA and inhibit transcription of the oncogene, inducing gene knockdown and subsequent apoptosis in cancer cells. Traditionally, siRNAs have faced clinical limitations due their lack of tissue specificity, rapid degradation within the body, and immune activation. However, the synthetic siRNAs herein contain novel chemical modifications that confer drug-like properties, which will protect them from in vivo degradation and displace the need for a nanocarrier or other delivery system.

Accordingly, one aspect of the invention relates to a double stranded RNA molecule comprising an antisense strand and a sense strand, wherein the nucleotide sequence of the antisense strand is complementary to a region of the nucleotide sequence of a c-Myc gene, the region consisting essentially of about 18 to about 25 consecutive nucleotides: wherein the double stranded RNA molecule inhibits expression of a c-Myc gene. The RNA molecules provide decreased expression of c-Myc in a cell as compared to cells without the RNA molecules (e.g., a control cell or nontransformed cell). In some embodiments, expression of c-Myc is inhibited by at least about 50%, e.g., at least about 50%, 60%, 70%, 80%, 90%, 95%, or more.

The double stranded RNA molecule can comprise, consist essentially of, or consist of about 18 to about 25 nucleotides (e.g., 18, 19, 20, 21, 22, 23, 24, or 25 or any range therein). Additional nucleotides can be added at the 3′ end, the 5′ end or both the 3′ and 5′ ends to facilitate manipulation of the RNA molecule but that do not materially affect the basic characteristics or function of the double stranded RNA molecule in RNA interference (RNAi). Additionally, one or two nucleotides can be deleted from one or both ends of any of the sequences disclosed herein that do not materially affect the basic characteristics or function of the double stranded RNA molecule in RNAi. The term “materially affect” as used herein refers to a change in the ability to inhibit expression of the protein encoded by the mRNA by no more than about 50%, e.g., no more than about 50%, 45%, 40%, 35%, 30%, 25%, 20%, 15%, 10%, or less. Such additional nucleotides can be nucleotides that extend the complementarity of the antisense strand along the target sequence and/or such nucleotides can be nucleotides that facilitate manipulation of the RNA molecule or a nucleic acid molecule encoding the RNA molecule, as would be known to one of skill in the art. For example, a TT overhang at the 3′ end may be present, which is used to stabilize the siRNA duplex and does not affect the specificity of the siRNA.

The dsRNA of the invention may optionally comprise a single stranded overhang at either or both ends. The double-stranded structure may be formed by a single self-complementary RNA strand (i.e., forming a hairpin loop) or two complementary RNA strands. RNA duplex formation may be initiated either inside or outside the cell. When the dsRNA of the invention forms a hairpin loop, it may optionally comprise an intron and/or a nucleotide spacer, which is a stretch of nucleotides between the complementary RNA strands, to stabilize the hairpin sequence in cells. The RNA may be introduced in an amount that allows delivery of at least one copy per cell. Higher doses of double-stranded material may yield more effective inhibition.

In particular embodiments, the present invention provides double stranded RNA containing a nucleotide sequence that is fully complementary to a region of the target gene for inhibition. However, it is to be understood that 100% complementarity between the antisense strand of the double stranded RNA molecule and the target sequence is not required to practice the present invention. Thus, sequence variations that might be expected due to genetic mutation, strain polymorphism, or evolutionary divergence can be tolerated. RNA sequences with insertions, deletions, and single point mutations relative to the target sequence may also be effective for inhibition.

In some embodiments, the nucleotide sequence of the sense strand comprises a nucleotide sequence that is at least about 80% identical to the nucleotide sequence of any of SEQ ID NOS:1-9, e.g., at least about 80%, 85%, 90%, 91%, 92%, 93%, 94%, 95%, 96%, 97%, 98%, 99%, or more identical to the nucleotide sequence of any of SEQ ID NOS:1-9. In some embodiments, the nucleotide sequence of the sense strand comprises, consists essentially of, or consists of the nucleotide sequence of any of SEQ ID NOS: 1-9.

SEQ ID NO: 1
GAGGAUAUCUGGAAGAAAU
SEQ ID NO: 2
GAGAACAGUUGAAACACAA
SEQ ID NO: 3
AACACAAACUUGAACAGCU
SEQ ID NO: 4
AAGAAGAUGAGGAAGAAAU
SEQ ID NO: 5
CACAGCCCACUGGUCCUCAAGA
SEQ ID NO: 6
AAGAGGCGAACACACAACGUC
SEQ ID NO: 7
CAGAUCAGCAACAACCG
SEQ ID NO: 8
GAGACCUUCAUCAAAAACAUCAUCA
SEQ ID NO: 9
GAGCUAAAACGGAGCUUUU

In some embodiments, the nucleotide sequence of the antisense strand comprises a nucleotide sequence that is at least about 80% identical to the nucleotide sequence of any of SEQ ID NOS:19-27, e.g., at least about 80%, 85%, 90%, 91%, 92%, 93%, 94%, 95%, 96%, 97%, 98%, 99%, or more identical to the nucleotide sequence of any of SEQ ID NOS: 19-27. In some embodiments, the nucleotide sequence of the antisense strand comprises, consists essentially of, or consists of the nucleotide sequence of any of SEQ ID NOS: 19-27.

SEQ ID NO: 19
AUUUCUUCCAGAUAUCCUC
SEQ ID NO: 20
UUGUGUUUCAACUGUUCUC
SEQ ID NO: 21
AGCUGUUCAAGUUUGUGUU
SEQ ID NO: 22
AUUUCUUCCUCAUCUUCUU
SEQ ID NO: 23
UCUUGAGGACCAGUGGGCUGUG
SEQ ID NO: 24
GACGUUGUGUGUUCGCCUCUU
SEQ ID NO: 25
CGGUUGUUGCUGAUCUG
SEQ ID NO: 26
UGAUGAUGUUUUUGAUGAAGGUCUC
SEQ ID NO: 27
AAAAGCUCCGUUUUAGCUC

In some embodiments, one or both of the sense strand and the antisense strand comprises a TT overhang at the 3′ end. Thus, in some embodiments, the sense strand comprises a nucleotide sequence that is at least about 80% identical to the nucleotide sequence of any of SEQ ID NOS:10-18, e.g., at least about 80%, 85%, 90%, 91%, 92%, 93%, 94%, 95%, 96%, 97%, 98%, 99%, or more identical to the nucleotide sequence of any of SEQ ID NOS: 10-18. In some embodiments, the nucleotide sequence of the sense strand comprises, consists essentially of, or consist of the nucleotide sequence of any of SEQ ID NOS: 10-18.

SEQ ID NO: 10
GAGGAUAUCUGGAAGAAAUdTdT
SEQ ID NO: 11
GAGAACAGUUGAAACACAAdTdT
SEQ ID NO: 12
AACACAAACUUGAACAGCUdTdT
SEQ ID NO: 13
AAGAAGAUGAGGAAGAAAUdTdT
SEQ ID NO: 14
CACAGCCCACUGGUCCUCAAGAdTdT
SEQ ID NO: 15
AAGAGGCGAACACACAACGUCdTdT
SEQ ID NO: 16
CAGAUCAGCAACAACCGdTdT
SEQ ID NO: 17
GAGACCUUCAUCAAAAACAUCAUCAdTdT
SEQ ID NO: 18
GAGCUAAAACGGAGCUUUUdTdT

In some embodiments, the nucleotide sequence of the antisense strand comprises a nucleotide sequence that is at least about 80% identical to the nucleotide sequence of any of SEQ ID NOS:28-36, e.g., at least about 80%, 85%, 90%, 91%, 92%, 93%, 94%, 95%, 96%, 97%, 98%, 99%, or more identical to the nucleotide sequence of any of SEQ ID NOS: 28-36. In some embodiments, the nucleotide sequence of the antisense strand comprises, consists essentially of, or consist of the nucleotide sequence of any of SEQ ID NOS: 28-36.

SEQ ID NO: 28
AUUUCUUCCAGAUAUCCUCdTdT
SEQ ID NO: 29
UUGUGUUUCAACUGUUCUCdTdT
SEQ ID NO: 30
AGCUGUUCAAGUUUGUGUUdTdT
SEQ ID NO: 31
AUUUCUUCCUCAUCUUCUUdTdT
SEQ ID NO: 32
UCUUGAGGACCAGUGGGCUGUGdTdT
SEQ ID NO: 33
GACGUUGUGUGUUCGCCUCUUdTdT
SEQ ID NO: 34
CGGUUGUUGCUGAUCUGdTdT
SEQ ID NO: 35
UGAUGAUGUUUUUGAUGAAGGUCUCdTdT
SEQ ID NO: 36
AAAAGCUCCGUUUUAGCUCdTdT

In some embodiments of this invention, the sense strand of the double stranded RNA molecule can be fully complementary to the antisense strand or the sense strand can be substantially complementary or partially complementary to the antisense strand. By substantially or partially complementary is meant that the sense strand and the antisense strand can be mismatched at about 1, 2, 3, 4, 5, 6, 7, 8, 9, or 10 nucleotide pairings. Such mismatches can be introduced into the sense strand sequence, e.g., near the 3′ end, to enhance processing of the double stranded RNA molecule by Dicer, to duplicate a pattern of mismatches in a siRNA molecule inserted into a chimeric nucleic acid molecule or artificial microRNA precursor molecule of this invention, and the like, as would be known to one of skill in the art. Such modification will weaken the base pairing at one end of the duplex and generate strand asymmetry, therefore enhancing the chance of the antisense strand, instead of the sense strand, being processed and silencing the intended gene (Geng and Ding “Double-mismatched siRNAs enhance selective gene silencing of a mutant ALS-causing Allelel” Acta Pharmacol. Sin. 29:211-216 (2008): Schwarz et al. “Asymmetry in the assembly of the RNAi enzyme complex” Cell 115:199-208 (2003)).

The double stranded RNA molecule of the invention may be in the form of any type of RNA interference molecule known in the art. In some embodiments, the double stranded RNA molecule is a small interfering RNA (siRNA) molecule. In other embodiments, the double stranded RNA molecule is a short hairpin RNA (shRNA) molecule. In other embodiments, the double stranded RNA molecule is part of a microRNA precursor molecule.

### Chemically-Modified siRNAs

One aspect of the invention relates to a siRNA molecule targeted to a c-Myc mRNA of the invention, wherein the siRNA comprises as least one chemical modification. In some embodiments, the siRNA molecule is fully chemically modified. The term “fully chemically-modified” means that every nucleotide in the siRNA contains a chemical modification. In some embodiments, each nucleotide in the siRNA molecule is modified with a 2′-O-methyl group or a 2′-fluoro group.

In certain embodiments, at least 1, 2, 3, 4, 5, 6, 7, 8, 9, 10, 11, 12, 13, 14, 15, 16, 17, 18, or 19 of the nucleotide linkages in the siRNA are chemically modified. In some embodiments, the siRNA comprises at least one phosphorothioate linkage. In some embodiments, the siRNA comprises 1, 2, 3, 4, 5, 6, 7, 8, 9, 10, 11, 12, 13, 14, 15, 16, 17, 18, or 19 phosphorothioate linkages. In some embodiments, the siRNA comprises all phosphorothioate linkages.

In certain embodiments, the siRNA molecule comprising at least one chemical modification comprises a sense strand and an antisense strand, wherein the siRNA molecule comprises one of the following pairs of sequences:
sense strand of SEQ ID NO:1 and antisense strand of SEQ ID NO:19;
sense strand of SEQ ID NO:2 and antisense strand of SEQ ID NO:20;
sense strand of SEQ ID NO:3 and antisense strand of SEQ ID NO:21:
sense strand of SEQ ID NO:4 and antisense strand of SEQ ID NO:22:
sense strand of SEQ ID NO:5 and antisense strand of SEQ ID NO:23;
sense strand of SEQ ID NO:6 and antisense strand of SEQ ID NO:24:
sense strand of SEQ ID NO:7 and antisense strand of SEQ ID NO:25:
sense strand of SEQ ID NO:8 and antisense strand of SEQ ID NO:26: or
sense strand of SEQ ID NO:9 and antisense strand of SEQ ID NO:27.

In some embodiments, the siRNA comprises, consists essentially of, or consists of a sequence at least 90% identical to one of SEQ ID NOS:1-9 and 19-27, e.g., at least 90%, 91%, 92%, 93%, 94%, 95%, 96%, 97%, 98%, or 99% identical.

TABLE 1
Unmodified siRNA sequences

| | | SEQ |
|---|---|---|
| Strand | Sequence (5′-3′) | ID NO |
| S | GAGGAUAUCUGGAAGAAAU | 1 |
| AS | AUUUCUUCCAGAUAUCCUC | 19 |
| S | GAGAACAGUUGAAACACAA | 2 |
| AS | UUGUGUUUCAACUGUUCUC | 20 |
| S | AACACAAACUUGAACAGCU | 3 |
| AS | AGCUGUUCAAGUUUGUGUU | 21 |
| S | AAGAAGAUGAGGAAGAAAU | 4 |
| AS | AUUUCUUCCUCAUCUUCUU | 22 |
| S | CACAGCCCACUGGUCCUCAAGA | 5 |
| AS | UCUUGAGGACCAGUGGGCUGUG | 23 |
| S | AAGAGGCGAACACACAACGUC | 6 |
| AS | GACGUUGUGUGUUCGCCUCUU | 24 |
| S | CAGAUCAGCAACAACCG | 7 |
| AS | CGGUUGUUGCUGAUCUG | 25 |
| S | GAGACCUUCAUCAAAAACAUCAUCA | 8 |
| AS | UGAUGAUGUUUUUGAUGAAGGUCUC | 26 |
| S | GAGCUAAAACGGAGCUUUU | 9 |
| AS | AAAAGCUCCGUUUUAGCUC | 27 |

S-sense strand
AS-antisense strand

In certain embodiments, the siRNA molecule is fully chemically modified and comprises a sense strand and an antisense strand, wherein the siRNA molecule comprises one of the following pairs of sequences:
sense strand of SEQ ID NO:37 and antisense strand of SEQ ID NO:38;
sense strand of SEQ ID NO:39 and antisense strand of SEQ ID NO:40;
sense strand of SEQ ID NO:41 and antisense strand of SEQ ID NO:42;
sense strand of SEQ ID NO:43 and antisense strand of SEQ ID NO:44; or
sense strand of SEQ ID NO:45 and antisense strand of SEQ ID NO:46.
In some embodiments, the siRNA comprises, consists essentially of, or consists of a sequence at least 90% identical to one of SEQ ID NOS:37-46, e.g., at least 90%, 91%, 92%, 93%, 94%, 95%, 96%, 97%, 98%, or 99% identical.

In some embodiments, the siRNA molecule can be combined in tandem (e.g., administered together by being covalently or non-covalently linked) with a second siRNA molecule targeted to human KRAS mRNA (e.g., a siRNA molecule as disclosed in US Patent Publication No. 2020/0248185, incorporated by reference herein in its entirety). In some embodiments, the siRNA molecule targeted to human KRAS mRNA is fully chemically modified and comprises a sense strand and an antisense strand, wherein the siRNA molecule comprises one of the following pairs of sequences:
sense strand of SEQ ID NO:47 and antisense strand of SEQ ID NO:48:
sense strand of SEQ ID NO:49 and antisense strand of SEQ ID NO:50.

In certain embodiments, at least 1, 2, 3, 4, 5, 6, 7, 8, 9, 10, 11, 12, 13, 14, 15, 16, 17, 18, or 19 of the nucleotide linkages in the KRAS-targeted siRNA are chemically modified. In some embodiments, the KRAS-targeted siRNA comprises at least one phosphorothioate linkage. In some embodiments, the KRAS-targeted siRNA comprises 1, 2, 3, 4, 5, 6, 7, 8, 9, 10, 11, 12, 13, 14, 15, 16, 17, 18, or 19 phosphorothioate linkages. In some embodiments, the KRAS-targeted siRNA comprises all phosphorothioate linkages.

In some embodiments, the KRAS-targeted siRNA comprises, consists essentially of, or consists of a sequence at least 90% identical to one of SEQ ID NOS:47-50, e.g., at least 90%, 91%, 92%, 93%, 94%, 95%, 96%, 97%, 98%, or 99% identical.

The inverted chimera siRNA molecule, double stranded RNA molecule, or chemically-modified siRNA molecule may be constructed using chemical synthesis and enzymatic ligation reactions by procedures known in the art. For example, an inverted chimera siRNA molecule, double stranded RNA, or chemically-modified siRNA molecule may be chemically synthesized using naturally occurring nucleotides or various modified nucleotides designed to increase the biological stability of the molecules or to increase the physical stability of the duplex formed between the inverted chimera siRNA molecule, double stranded RNA, or chemically-modified siRNA molecule and target nucleotide sequences, e.g., phosphorothioate derivatives and acridine substituted nucleotides can be used. Examples of modified nucleotides which can be used to generate the inverted chimera siRNA molecule, double stranded RNA, or chemically-modified siRNA molecule include, but are not limited to, 5-fluorouracil, 5-bromouracil, 5-chlorouracil, 5-iodouracil, hypoxanthine, xanthine, 4-acetylcytosine. 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine. 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxy carboxymethyluracil, 5-methoxyuracil, 2-methylthio-N6-isopenteny ladenine, uracil-5-oxyacetic acid(v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w; and 2,6-diaminopurine. Alternatively, the double stranded RNA can be produced using an expression vector into which a nucleic acid encoding the double stranded RNA has been cloned.

The inverted chimera siRNA molecule, double stranded RNA, or chemically- modified siRNA molecule can further include nucleotide sequences wherein at least one, or all, of the internucleotide bridging phosphate residues are modified phosphates, such as methyl phosphonates, methyl phosphonothioates, phosphoromorpholidates, phosphoropiperazidates and phosphoramidates. For example, every one or every other one of the internucleotide bridging phosphate residues can be modified as described. In another non-limiting example, the inverted chimera siRNA molecule, double stranded RNA, or chemically-modified siRNA molecule is a nucleotide sequence in which at least one, or all, of the nucleotides contain a 2′ lower alkyl moiety (e.g., C₁-C₄, linear or branched, saturated or unsaturated alkyl, such as methyl, ethyl, ethenyl, propyl, 1-propenyl, 2-propenyl, and isopropyl). In another example, one or more of the nucleotides may be a 2′-fluoro nucleotide, a 2-O-methyl nucleotide, or a locked nucleic acid nucleotide. For example, every one or every other one of the nucleotides can be modified as described. See also, Furdon et al., Nucleic Acids Res. 17:9193 (1989): Agrawal et al., Proc. Natl. Acad. Sci. USA 87:1401 (1990); Baker et al., Nucleic Acids Res. 18:3537 (1990); Sproat et al., Nucleic Acids Res. 17:3373 (1989): Walder and Walder, Proc. Natl. Acad. Sci. USA 85:5011 (1988): incorporated by reference herein in their entireties for their teaching of methods of making polynucleotide molecules, including those containing modified nucleotide bases).

The invention further relates to a nucleic acid construct comprising the RNA molecule of the invention. The invention further relates to a nucleic acid construct encoding the RNA molecule of the invention and a nucleic acid construct comprising the nucleic acid molecule encoding the RNA molecule. In each of these embodiments, the nucleic acid construct may be a vector or a plasmid, e.g., an expression vector.

Another aspect of the invention relates to a composition comprising the inverted chimera siRNA, molecule, RNA molecule, chemically-modified siRNA molecule, or nucleic acid construct of the invention and another component, e.g., a suitable carrier. In some embodiments, the composition comprises two or more of the inverted chimera siRNA. molecules, RNA molecules, chemically-modified siRNA molecules, or nucleic acid constructs of the invention, wherein the two or more inverted chimera siRNA molecules, RNA molecules, or chemically-modified siRNA molecule each comprise a different antisense strand. In certain embodiments, the two or more RNA molecules are present on the same nucleic acid construct, on different nucleic acid constructs or any combination thereof. In some embodiments, the composition is a pharmaceutical composition comprising the inverted chimera siRNA molecule(s), RNA molecule(s), chemically-modified siRNA molecule(s), or nucleic acid construct(s) of the invention and a pharmaceutically acceptable carrier.

It is understood that the compositions of this invention can comprise, consist essentially of, or consist of any of the inverted chimera siRNA molecules, RNA molecules, chemically-modified siRNA molecules, and nucleic acid constructs in any combination and in any ratio relative to one another. Furthermore, by “two or more” is meant 2, 3, 4, 5, 6, 7, 8, 9, 10, etc., up to a total number of inverted chimera siRNA molecules, RNA molecules, chemically-modified siRNA molecules, and nucleic acid constructs of this invention.

In some aspects of the invention, the composition or pharmaceutical composition further comprises additional components that enhance the delivery of the inverted chimera siRNA molecule(s), RNA molecule(s), chemically-modified siRNA molecule(s), or nucleic acid construct(s) of the invention to a subject, e.g., by enhancing the stability of the inverted chimera siRNA molecules, RNA molecule(s), chemically-modified siRNA molecule(s), or nucleic acid construct(s). In some embodiments, the additional component may be a particle, e.g., a microparticle or nanoparticle. In some embodiments, the particle is a lipid particle, e.g., a liposome, e.g., a microliposome or a nanoliposome. The liposome, microliposome, or nanoliposome may contain any components known in the art to be suitable for preparing liposomes. In some embodiments, the liposome comprises 1,2-dioleoyl-sn-glycero-3-phosphatidylcholine (DOPC). Liposomes may be prepared by methods known in the art, e.g., as described in Pecot et al., Mol. Cancer Ther. 13:2876 (2014), incorporated by reference herein in its entirety. In some embodiments, the RNA molecule is formed into a stable nucleic acid lipid particle (SNALP), e.g., using particles such as those provided by Arbutus Biopharma (Doylestown, PA). In certain embodiments, the lipid particle comprises, consists essentially of, or consists of cholesterol, 1,2-distearoyl-sn-glycero-3-phosphocholine (DSPC), PEG-CDMA or PEG-cDSA, and 1,2-dilinoleyloxy-3-(N,N-dimethyl)aminopropane (DLinDMA) (see Judge et al., J. Clin. Invest. 119:661 (2009)). In some embodiments, the lipid particle comprises two or more of the inverted chimera siRNA molecules, RNA molecules, or chemically-modified siRNA molecules of the invention. In some embodiments, the additional component is a targeted delivery moiety to which the inverted chimera siRNA molecules, RNA molecule(s), chemically-modified siRNA molecule(s), or nucleic acid construct(s) or covalently or noncovalently conjugated, e.g., ligands, aptamers, or monoclonal antibodies.

The present invention encompasses cells comprising the RNA molecules and/or nucleic acid constructs of the invention. Thus, in some embodiments, the present invention provides a transformed cell comprising a RNA molecule and/or a nucleic acid construct and/or a composition of this invention, wherein the transformed cell has reduced expression of c-Myc as compared to a control cell.

### Methods

Various methods are provided herein, employing the nucleic acid molecules, nucleic acid constructs, and/or compositions of this invention. Thus, in one aspect, the present invention provides a method of inhibiting expression of a target gene in a cell, the method comprising contacting the cell with the RNA molecule, inverted chimera siRNA molecule, chemically-modified siRNA molecule, nucleic acid construct, composition, and/or pharmaceutical composition of the invention, thereby inhibiting expression of the target gene in the cell.

Another aspect of the invention relates to a method of treating a disorder associated with a target gene in a subject in need thereof, the method comprising delivering to the subject the RNA molecule, inverted chimera siRNA molecule, chemically-modified siRNA molecule, nucleic acid construct, composition, and/or pharmaceutical composition of the invention, thereby treating the disorder in the subject. A disorder associated with a target gene is a disorder in which expression or over-expression of the target gene causes the disorder or causes one or more symptoms of the disorder or is expressed by an organism causing the disorder.

The present invention further provides a method of inhibiting expression of a human c-Myc gene, the method comprising contacting the cell with the RNA molecule, inverted chimera siRNA molecule, chemically-modified siRNA molecule, nucleic acid construct, composition, and/or pharmaceutical composition of the invention, thereby inhibiting expression of the human c-Myc gene in the cell.

Also provided herein is a method of treating cancer in a subject in need thereof, wherein the cancer comprises over-expression of a human c-Myc gene, the method comprising delivering to the subject the RNA molecule, inverted chimera siRNA molecule, chemically-modified siRNA molecule, nucleic acid construct, composition, and/or pharmaceutical composition of the invention, thereby treating cancer in the subject. A cancer comprising over-expression of a human c-Myc gene is a cancer, e.g., a tumor, in which one or more cells over-express the c-Myc gene.

In one embodiment of each of these aspects, the subject may be one that has been diagnosed with a disorder, e.g., cancer. In another embodiment, the subject may be one that is at risk of developing a disorder, e.g., cancer (e.g., predisposed due to hereditary factors, smoking, viral infection, exposure to chemicals, etc.). In a further embodiment, the subject may be one that is infected with a pathogen. In a further embodiment, the subject may be one that has been identified as over-expressing a target gene, e.g., a c-Myc gene, and has or has not been diagnosed with a disorder, e.g., cancer.

The double stranded RNA, inverted chimera siRNA molecule, or chemically- modified siRNA molecule of the invention can be delivered directly into a cell by any method known in the art, e.g., by transfection or microinjection, e.g., as part of a composition comprising lipid particles. In other embodiments, the double stranded RNA can be delivered to a subject in the form of polynucleotides encoding the RNA to produce expression of the double stranded RNA within the cells of the subject. Those skilled in the art will appreciate that the isolated polynucleotides encoding the RNAs of the invention will typically be associated with appropriate expression control sequences, e.g., transcription/translation control signals and polyadenylation signals.

It will further be appreciated that a variety of promoter/enhancer elements can be used depending on the level and tissue-specific expression desired. The promoter can be constitutive or inducible, depending on the pattern of expression desired. The promoter can be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) of interest.

To illustrate, the RNA coding sequence can be operatively associated with a cytomegalovirus (CMV) major immediate-early promoter, an albumin promoter, an Elongation Factor 1-α (EF1-α) promoter, a PγK promoter, a MFG promoter, or a Rous sarcoma virus promoter.

Inducible promoter/enhancer elements include hormone-inducible and metal- inducible elements, and other promoters regulated by exogenously supplied compounds, including without limitation, the zinc-inducible metallothionein (MT) promoter: the dexamethasone (Dex)-inducible mouse mammary tumor virus (MMTV) promoter; the T7 polymerase promoter system (see WO 98/10088): the ecdysone insect promoter (No et al., Proc. Natl. Acad. Sci. USA 93:3346 (1996)); the tetracycline-repressible system (Gossen et al, Proc. Natl. Acad. Sci. USA 89:5547 (1992)); the tetracycline-inducible system (Gossen et al., Science 268:1766 (1995); see also Harvey et al., Curr. Opin. Chem. Biol. 2:512 (1998)); the RU486-inducible system (Wang et al., Nat. Biotech. 15:239 (1997); Wang et al., Gene Ther., 4:432 (1997)); and the rapamycin-inducible system (Magari et al., J. Clin. Invest. 100:2865 (1997)).

Other tissue-specific promoters or regulatory promoters include, but are not limited to, promoters that typically confer tissue-specificity in neurons. These include, but are not limited to, promoters for synapsin 1, tubulin α1, platelet-derived growth factor B-chain, tyrosine hydroxylase, neuron-specific enolase, and neurofilaments. Skeletal muscle cell promoters include, but are not limited to, promoters for β-actin, Pitx3, creatine kinase, and myosin light chain. Cardiac muscle cell promoters include, but are not limited to, promoters for cardiac actin, cardiac troponin T, troponin C, myosin light chain-2, and α-myosin heavy chain. Islet (beta) cell promoters include, but are not limited to, glucokinase, gastrin, insulin, and islet amyloid polypeptide.

Moreover, specific initiation signals are generally required for efficient translation of inserted RNA coding sequences. These translational control sequences, which can include the ATG initiation codon and adjacent sequences, can be of a variety of origins, both natural and synthetic.

The isolated nucleic acid encoding the double stranded RNA can be incorporated into an expression vector. Expression vectors compatible with various host cells are well known in the art and contain suitable elements for transcription and translation of nucleic acids. Typically, an expression vector contains an “expression cassette,” which includes, in the 5′ to 3′ direction, a promoter, a coding sequence encoding a double stranded RNA operatively associated with the promoter, and, optionally, a termination sequence including a stop signal for RNA polymerase and a polyadenylation signal for polyadenylase.

Non-limiting examples of animal and mammalian promoters known in the art include, but are not limited to, the SV40 early (SV40e) promoter region, the promoter contained in the 3′ long terminal repeat (LTR) of Rous sarcoma virus (RSV), the promoters of the ElA or major late promoter (MLP) genes of adenoviruses (Ad), the cytomegalovirus (CMV) early promoter, the herpes simplex virus (HSV) thymidine kinase (TK) promoter, baculovirus IE1 promoter, elongation factor 1 alpha (EF1) promoter, phosphoglycerate kinase (PGK) promoter, ubiquitin (Ubc) promoter, an albumin promoter, the regulatory sequences of the mouse metallothionein-L promoter and transcriptional control regions, the ubiquitous promoters (HPRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), and pathogenesis and/or disease- related promoters. In addition, any of these expression sequences of this invention can be modified by addition of enhancer and/or regulatory sequences and the like.

Enhancers that may be used in embodiments of the invention include but are not limited to: an SV40 enhancer, a cytomegalovirus (CMV) enhancer, an elongation factor I (EF1) enhancer, yeast enhancers, viral gene enhancers, and the like.

Termination control regions, i.e., terminator or polyadenylation sequences, may be derived from various genes native to the preferred hosts. In some embodiments of the invention, the termination control region may comprise or be derived from a synthetic sequence, a synthetic polyadenylation signal, an SV40 late polyadenylation signal, an SV40 polyadenylation signal, a bovine growth hormone (BGH) polyadenylation signal, viral terminator sequences, or the like.

It will be apparent to those skilled in the art that any suitable vector can be used to deliver the polynucleotide to a cell or subject. The vector can be delivered to cells in vivo. In other embodiments, the vector can be delivered to cells ex vivo, and then cells containing the vector are delivered to the subject. The choice of delivery vector can be made based on a number of factors known in the art, including age and species of the target host, in vitro versus in vivo delivery, level and persistence of expression desired, intended purpose (e.g., for therapy or screening), the target cell or organ, route of delivery, size of the isolated polynucleotide, safety concerns, and the like.

Suitable vectors include, but are not limited to, plasmid vectors, viral vectors (e.g., retrovirus, alphavirus: vaccinia virus; adenovirus, adeno-associated virus and other parvoviruses, lentivirus, poxvirus, or herpes simplex virus), lipid vectors, poly-lysine vectors, synthetic polyamino polymer vectors, and the like.

Any viral vector that is known in the art can be used in the present invention. Protocols for producing recombinant viral vectors and for using viral vectors for nucleic acid delivery can be found in Ausubel et al., Current Protocols in Molecular Biology (Green Publishing Associates, Inc. and John Wiley & Sons, Inc., New York) and other standard laboratory manuals (e.g., Vectors for Gene Therapy. In: Current Protocols in Human Genetics. John Wiley and Sons, Inc.: 1997).

Non-viral transfer methods can also be employed. Many non-viral methods of nucleic acid transfer rely on normal mechanisms used by mammalian cells for the uptake and intracellular transport of macromolecules. In particular embodiments, non-viral nucleic acid delivery systems rely on endocytic pathways for the uptake of the nucleic acid molecule by the targeted cell. Exemplary nucleic acid delivery systems of this type include liposomal derived systems, poly-lysine conjugates, and artificial viral envelopes.

In particular embodiments, plasmid vectors are used in the practice of the present invention. For example, naked plasmids can be introduced into muscle cells by injection into the tissue. Expression can extend over many months, although the number of positive cells is typically low (Wolff et al., Science 247:247 (1989)). Cationic lipids have been demonstrated to aid in introduction of nucleic acids into some cells in culture (Felgner and Ringold, Nature 337:387 (1989)). Injection of cationic lipid plasmid DNA complexes into the circulation of mice has been shown to result in expression of the DNA in lung (Brigham et al., Am. J. Med. Sci. 298:278 (1989)). One advantage of plasmid DNA is that it can be introduced into non- replicating cells.

In a representative embodiment, a nucleic acid molecule (e.g., a plasmid) can be entrapped in a lipid particle bearing positive charges on its surface and, optionally, tagged with antibodies against cell surface antigens of the target tissue (Mizuno et al., No Shinkei Geka 20:547 (1992); PCT publication WO 91/06309; Japanese patent application 1047381: and European patent publication EP-A-43075).

Liposomes that consist of amphiphilic cationic molecules are useful as non-viral vectors for nucleic acid delivery in vitro and in vivo (reviewed in Crystal, Science 270:404 (1995); Blaese et al., Cancer Gene Ther. 2:291 (1995); Behr et al., Bioconjugate Chem. 5:382 (1994); Remy et al., Bioconjugate Chem. 5:647 (1994); and Gao et al., Gene Therapy 2:710 (1995)). The positively charged liposomes are believed to complex with negatively charged nucleic acids via electrostatic interactions to form lipid:nucleic acid complexes. The lipid:nucleic acid complexes have several advantages as nucleic acid transfer vectors. Unlike viral vectors, the lipid:nucleic acid complexes can be used to transfer expression cassettes of essentially unlimited size. Since the complexes lack proteins, they can evoke fewer immunogenic and inflammatory responses. Moreover, they cannot replicate or recombine to form an infectious agent and have low integration frequency. A number of publications have demonstrated that amphiphilic cationic lipids can mediate nucleic acid delivery in vivo and in vitro (Felgner et al., Proc. Natl. Acad. Sci. USA 84:7413 (1987); Loeffler et al., Meth. Enzymol. 217:599 (1993); Felgner et al., J. Biol. Chem. 269:2550 (1994)).

Several groups have reported the use of amphiphilic cationic lipid:nucleic acid complexes for in vivo transfection both in animals and in humans (reviewed in Gao et al., Gene Therapy 2:710 (1995): Zhu et al., Science 261:209 (1993); and Thierry et al., Proc. Natl. Acad. Sci. USA 92:9742 (1995)). U.S. Pat. No. 6,410,049 describes a method of preparing cationic lipid:nucleic acid complexes that have a prolonged shelf life.

Nuclear localization signals can also be used to enhance the targeting of the double stranded RNA or expression vector into the proximity of the nucleus and/or its entry into the nucleus. Such nuclear localization signals can be a protein or a peptide such as the SV40) large Tag NLS or the nucleoplasmin NLS. These nuclear localization signals interact with a variety of nuclear transport factors such as the NLS receptor (karyopherin alpha) which then interacts with karyopherin beta.

Expression vectors can be designed for expression of double stranded RNA in prokaryotic or eukaryotic cells. For example, double stranded RNA can be expressed in bacterial cells such as E. coli, insect cells (e.g., the baculovirus expression system), yeast cells, plant cells or mammalian cells. Some suitable host cells are discussed further in Goeddel, Gene Expression Technology: Methods in Enzymology 185, Academic Press, San Diego, Calif. (1990). Examples of bacterial vectors include, but are not limited to, pQE70, pQE60, pQE-9) (Qiagen), pBS, pD10, phagescript, psiX174, pbluescript SK, pbsks, pNH8A, pNH16a, pNH18A, pNH46A (Stratagene); ptrc99a, pKK223-3, pKK233-3, pDR540), and pRIT5 (Pharmacia). Examples of vectors for expression in the yeast S. cerevisiae include pYepSecl (Baldari et al., EMBO J. 6:229 (1987)), pMFa (Kurjan and Herskowitz, Cell 30:933 (1982)), pJRY88 (Schultz et al., Gene 54:113 (1987)), and pYES2 (Invitrogen Corporation, San Diego, Calif.). Non-limiting examples of baculovirus vectors available for expression of nucleic acids to produce proteins in cultured insect cells (e.g., Sf 9 cells) include the pAc series (Smith et al., Mol. Cell. Biol. 3:2156 (1983)) and the pVL series (Lucklow and Summers Virology 170:31 (1989)).

Examples of mammalian expression vectors include pWLNEO, pSV2CAT, pOG44, pXT1, pSG (Stratagene) pSVK3, PBPV, pMSG, PSVL (Pharmacia), pCDM8 (Seed, Nature 329:840 (1987)) and pMT2PC (Kaufman et al., EMBO J. 6:187 (1987)). When used in mammalian cells, the expression vector's control functions are often provided by viral regulatory elements. For example, commonly used promoters are derived from polyoma, adenovirus 2, cytomegalovirus and Simian Virus 40.

Viral vectors have been used in a wide variety of gene delivery applications in cells, as well as living animal subjects. Viral vectors that can be used include, but are not limited to, retrovirus, lentivirus, adeno-associated virus, poxvirus, alphavirus, baculovirus, vaccinia virus, herpes virus, Epstein-Barr virus, adenovirus, geminivirus, and caulimovirus vectors. Non-limiting examples of non-viral vectors include plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (delivery to specific tissues, duration of expression, etc.).

In addition to the regulatory control sequences discussed above, the recombinant expression vector can contain additional nucleotide sequences. For example, the recombinant expression vector can encode a selectable marker gene to identify host cells that have incorporated the vector.

Vector DNA can be introduced into prokaryotic or eukaryotic cells via conventional transformation or transfection techniques. As used herein, the terms “transformation” and “transfection” refer to a variety of art-recognized techniques for introducing foreign nucleic acids (e.g., DNA and RNA) into a host cell, including, but are not limited to, calcium phosphate or calcium chloride co-precipitation, DEAE-dextran-mediated transfection, lipofection, electroporation, microinjection, DNA-loaded liposomes, lipofectamine-DNA complexes, cell sonication, gene bombardment using high velocity microprojectiles, and viral-mediated transfection. Suitable methods for transforming or transfecting host cells can be found in Sambrook et al., Molecular Cloning: A Laboratory Manual 2nd Ed. (Cold Spring Harbor, NY, 1989), and other laboratory manuals.

If stable integration is desired, often only a small fraction of cells (in particular, mammalian cells) integrate the foreign DNA into their genome. In order to identify and select integrants, a nucleic acid that encodes a selectable marker (e.g., resistance to antibiotics) can be introduced into the host cells along with the nucleic acid of interest. Preferred selectable markers include those that confer resistance to drugs, such as G418, hygromycin and methotrexate. Nucleic acids encoding a selectable marker can be introduced into a host cell on the same vector as that comprising the nucleic acid of interest or can be introduced on a separate vector. Cells stably transfected with the introduced nucleic acid can be identified by drug selection (e.g., cells that have incorporated the selectable marker gene will survive, while the other cells die).

In one embodiment, the double stranded RNA, inverted chimera siRNA molecule, or chemically-modified siRNA molecule of the invention is administered directly to the subject. Generally, the compounds of the invention will be suspended in a pharmaceutically- acceptable carrier (e.g., physiological saline) and administered orally, topically, or by intravenous infusion, or injected subcutaneously, intramuscularly, intracranially, intrathecally, intraperitoneally, intrarectally, intravaginally, intranasally, intragastrically, intratracheally, or intrapulmonarily. They are preferably delivered directly to the site of the disease or disorder, such as the lung, intestine, or pancreas. The dosage required depends on the choice of the route of administration: the nature of the formulation: the nature of the patient's illness: the subject's size, weight, surface area, age, and sex: other drugs being administered: and the judgment of the attending physician. Suitable dosages are in the range of 0.01-100.0 μg/kg. Wide variations in the needed dosage are to be expected in view of the differing efficiencies of various routes of administration. For example, oral administration would be expected to require higher dosages than administration by i.v. injection (e.g., 2-, 3-, 4-, 6-, 8-, 10-, 20-, 50-, 100-, 150-, or more fold). Variations in these dosage levels can be adjusted using standard empirical routines for optimization as is well understood in the art. Administrations can be single or multiple. Encapsulation of the inhibitor in a suitable delivery vehicle (e.g., polymeric microparticles or implantable devices) may increase the efficiency of delivery, particularly for oral delivery.

According to certain embodiments, the double stranded RNA, inverted chimera siRNA molecule, or chemically-modified siRNA molecule can be targeted to specific cells or tissues in vivo. Targeting delivery vehicles, including liposomes and viral vector systems are known in the art. For example, a liposome can be directed to a particular target cell or tissue by using a targeting agent, such as an antibody, soluble receptor or ligand, incorporated with the liposome, to target a particular cell or tissue to which the targeting molecule can bind. Targeting liposomes are described, for example, in Ho et al., Biochemistry 25:5500 (1986); Ho et al., J. Biol. Chem. 262:13979 (1987); Ho et al., J. Biol. Chem. 262: 13973 (1987); and U.S. Pat. No. 4,957,735 to Huang et al., each of which is incorporated herein by reference in its entirety). Enveloped viral vectors can be modified to deliver a nucleic acid molecule to a target cell by modifying or substituting an envelope protein such that the virus infects a specific cell type. In adenoviral vectors, the gene encoding the attachment fibers can be modified to encode a protein domain that binds to a cell-specific receptor. Herpesvirus vectors naturally target the cells of the central and peripheral nervous system. Alternatively, the route of administration can be used to target a specific cell or tissue. For example, intracoronary administration of an adenoviral vector has been shown to be effective for the delivery of a gene to cardiac myocytes (Maurice et al., J. Clin. Invest. 104:21 (1999)). Intravenous delivery of cholesterol-containing cationic liposomes has been shown to preferentially target pulmonary tissues (Liu et al., Nature Biotechnol. 15:167 (1997)), and effectively mediate transfer and expression of genes in vivo. Other examples of successful targeted in vivo delivery of nucleic acid molecules are known in the art. Finally, a recombinant nucleic acid molecule can be selectively (i.e., preferentially, substantially exclusively) expressed in a target cell by selecting a transcription control sequence, and preferably, a promoter, which is selectively induced in the target cell and remains substantially inactive in non-target cells.

The double stranded RNA, inverted chimera siRNA molecule, or chemically- modified siRNA molecule of the present invention can optionally be delivered in conjunction with other therapeutic agents. The additional therapeutic agents can be delivered concurrently with the double stranded RNA, inverted chimera siRNA molecule, or chemically-modified siRNA molecule of the invention. As used herein, the word “concurrently” means sufficiently close in time to produce a combined effect (that is, concurrently can be simultaneously, or it can be two or more events occurring within a short time period before or after each other). In one embodiment, the double stranded RNA, inverted chimera siRNA molecule, or chemically-modified siRNA molecule of the invention are administered in conjunction with agents useful for treating cancer, such as: 1) vinca alkaloids (e.g., vinblastine, vincristine): 2) epipodophyllotoxins (e.g., etoposide and teniposide): 3) antibiotics (e.g., dactinomycin (actinomycin D), daunorubicin (daunomycin: rubidomycin), doxorubicin, bleomycin, plicamycin (mithramycin), and mitomycin (mitomycin C)): 4) enzymes (e.g., L-asparaginase): 5) biological response modifiers (e.g., interferon-alfa): 6) platinum coordinating complexes (e.g., cisplatin and carboplatin): 7) anthracenediones (e.g., mitoxantrone): 8) substituted ureas (e.g., hydroxyurea): 9) methylhydrazine derivatives (e.g., procarbazine (N-methylhydrazine: MIH)): 10) adrenocortical suppressants (e.g., mitotane (o.p′-DDD) and aminoglutethimide): 11) adrenocorticosteroids (e.g., prednisone): 12) progestins (e.g., hydroxyprogesterone caproate, medroxyprogesterone acetate, and megestrol acetate): 13) estrogens (e.g., diethylstilbestrol and ethinyl estradiol): 14) antiestrogens (e.g., tamoxifen): 15) androgens (e.g., testosterone propionate and fluoxymesterone): 16) antiandrogens (e.g., flutamide): and 17) gonadotropin- releasing hormone analogs (e.g., leuprolide). In another embodiment, the compounds of the invention are administered in conjunction with anti-angiogenesis agents, such as antibodies to VEGF (e.g., bevacizumab (AVASTIN), ranibizumab (LUCENTIS)) and other promoters of angiogenesis (e.g., bFGF, angiopoietin-1), antibodies to alpha-v/beta-3 vascular integrin (e.g., VITAXIN), angiostatin, endostatin, dalteparin, ABT-510, CNGRC peptide TNF alpha conjugate, cyclophosphamide, combretastatin A4 phosphate, dimethylxanthenone acetic acid, docetaxel, lenalidomide, enzastaurin, paclitaxel, paclitaxel albumin-stabilized nanoparticle formulation (Abraxane), soy isoflavone (Genistein), tamoxifen citrate, thalidomide, ADH-1 (EXHERIN), AG-013736, AMG-706, AZD2171, sorafenib tosylate, BMS-582664, CHIR-265, pazopanib, PI-88, vatalanib, everolimus, suramin, sunitinib malate, XL184, ZD6474, ATN-161, cilenigtide, and celecoxib, or any combination thereof.

The term “cancer,” as used herein, refers to any benign or malignant abnormal growth of cells. Examples include, without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is selected from the group of tumor-forming cancers.

### Pharmaceutical Compositions

As a further aspect, the invention provides pharmaceutical formulations and methods of administering the same to achieve any of the therapeutic effects (e.g., treatment of cancer) discussed above. The pharmaceutical formulation may comprise any of the reagents discussed above in a pharmaceutically acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

The double stranded RNA, inverted chimera siRNA molecule, chemically-modified siRNA molecule, or nucleic acid construct of the invention can be formulated for administration in a pharmaceutical carrier in accordance with known techniques. See. e.g., Remington, The Science And Practice of Pharmacy (9^{th} Ed. 1995). In the manufacture of a pharmaceutical formulation according to the invention, the double stranded RNA, inverted chimera siRNA molecule, or chemically-modified siRNA molecule (including the physiologically acceptable salts thereof) is typically admixed with, inter alia, an acceptable carrier. The carrier can be a solid or a liquid, or both, and is preferably formulated with the double stranded RNA, inverted chimera siRNA molecule, or chemically-modified siRNA molecule as a unit-dose formulation, for example, a tablet, which can contain from 0.01 or 0).5% to 95% or 99% by weight of the double stranded RNA, inverted chimera siRNA molecule, or chemically-modified siRNA molecule. One or more double stranded RNAs, inverted chimera siRNA molecules, or chemically-modified siRNA molecules can be incorporated in the formulations of the invention, which can be prepared by any of the well-known techniques of pharmacy.

A further aspect of the invention is a method of treating subjects in vivo, comprising administering to a subject a pharmaceutical composition comprising a double stranded RNA, inverted chimera siRNA molecule, or chemically-modified siRNA molecule of the invention in a pharmaceutically acceptable carrier, wherein the pharmaceutical composition is administered in a therapeutically effective amount. Administration of the double stranded RNA, inverted chimera siRNA molecule, or chemically-modified siRNA molecule of the present invention to a human subject or an animal in need thereof can be by any means known in the art for administering compounds.

Non-limiting examples of formulations of the invention include those suitable for oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intracranial, intrathecal, and inhalation administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor). The most suitable route in any given case will depend on the nature and severity of the condition being treated and on the nature of the particular compound which is being used. In some embodiments, it may be desirable to deliver the formulation locally to avoid any side effects associated with systemic administration. For example, local administration can be accomplished by direct injection at the desired treatment site, by introduction intravenously at a site near a desired treatment site (e.g., into a vessel that feeds a treatment site). In some embodiments, the formulation can be delivered locally to ischemic tissue. In certain embodiments, the formulation can be a slow release formulation, e.g., in the form of a slow release depot.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Compounds can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth: and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect of the present invention, there is provided an injectable, stable, sterile composition comprising a compound of the invention, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is pharmaceutically acceptable can be employed in sufficient quantity to emulsify the compound or salt in an aqueous carrier. One such useful emulsifying agent is phosphatidyl choline.

Formulations suitable for rectal administration are preferably presented as unit dose suppositories. These can be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the compound. Suitable formulations comprise citrate or bis\tris buffer (pH 6) or ethanol/water and contain from 0.1 to 0.2M of the compound.

The compound can alternatively be formulated for nasal administration or otherwise administered to the lungs of a subject by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987): Gonda (1990) Critical Reviews in Therapeutic Drug Carrier Systems 6:273-313: and Raeburn et al., J. Pharmacol. Toxicol. Meth. 27:143 (1992). Aerosols of liquid particles comprising the compound can be produced by any suitable means, such as with a pressure-driven aerosol nebulizer or an ultrasonic nebulizer, as is known to those of skill in the art. See. e.g., U.S. Pat. No. 4,501,729. Aerosols of solid particles comprising the compound can likewise be produced with any solid particulate medicament aerosol generator, by techniques known in the pharmaceutical art.

Alternatively, one can administer the compound in a local rather than systemic manner, for example, in a depot or sustained-release formulation.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same can be incorporated into lipid vesicles. In such an instance, due to the water solubility of the compound or salt, the compound or salt will be substantially entrained within the hydrophilic center or core of the liposomes. The lipid layer employed can be of any conventional composition and can either contain cholesterol or can be cholesterol-free. When the compound or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations containing the compounds disclosed herein or salts thereof, can be lyophilized to produce a lyophilizate which can be reconstituted with a pharmaceutically acceptable carrier, such as water, to regenerate a liposomal suspension.

In the case of water-insoluble compounds, a pharmaceutical composition can be prepared containing the water-insoluble compound, such as for example, in an aqueous base emulsion. In such an instance, the composition will contain a sufficient amount of pharmaceutically acceptable emulsifying agent to emulsify the desired amount of the compound. Particularly useful emulsifying agents include phosphatidyl cholines and lecithin.

In particular embodiments, the compound is administered to the subject in a therapeutically effective amount, as that term is defined above. Dosages of pharmaceutically active compounds can be determined by methods known in the art, see, e.g, Remington's Pharmaceutical Sciences (Maack Publishing Co., Easton, Pa). The therapeutically effective dosage of any specific compound will vary somewhat from compound to compound, and patient to patient, and will depend upon the condition of the patient and the route of delivery. As a general proposition, a dosage from about 0.001 to about 50 mg/kg will have therapeutic efficacy, with all weights being calculated based upon the weight of the compound, including the cases where a salt is employed. Toxicity concerns at the higher level can restrict intravenous dosages to a lower level such as up to about 10 mg/kg, with all weights being calculated based upon the weight of the compound, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Particular dosages are about 1 μmol/kg to 50 μmol/kg, and more particularly to about 22 μmol/kg and to 33 μmol/kg of the compound for intravenous or oral administration, respectively.

In particular embodiments of the invention, more than one administration (e.g., two, three, four, or more administrations) can be employed over a variety of time intervals (e.g., hourly, daily, weekly, monthly, etc.) to achieve therapeutic effects.

The present invention finds use in veterinary and medical applications. Suitable subjects include both avians and mammals, with mammals being preferred. The term “avian” as used herein includes, but is not limited to, chickens, ducks, geese, quail, turkeys, and pheasants. The term “mammal” as used herein includes, but is not limited to, humans, bovines, ovines, caprines, equines, felines, canines, lagomorphs, etc. Human subjects include neonates, infants, juveniles, and adults. In other embodiments, the subject is an animal model of cancer. In certain embodiments, the subject has or is at risk for cancer.

The following examples are not intended to limit the scope of the claims to the invention, but are rather intended to be exemplary of certain embodiments. Any variations in the exemplified methods that occur to the skilled artisan are intended to fall within the scope of the present invention. As will be understood by one skilled in the art, there are several embodiments and elements for each aspect of the claimed invention, and all combinations of different elements are hereby anticipated, so the specific combinations exemplified herein are not to be construed as limitations in the scope of the invention as claimed. If specific elements are removed or added to the group of elements available in a combination, then the group of elements is to be construed as having incorporated such a change.

### EXAMPLE 1

### Synthetic siRNAs Show Decrease of c-MYC mRNA Transcripts and Protein Expression

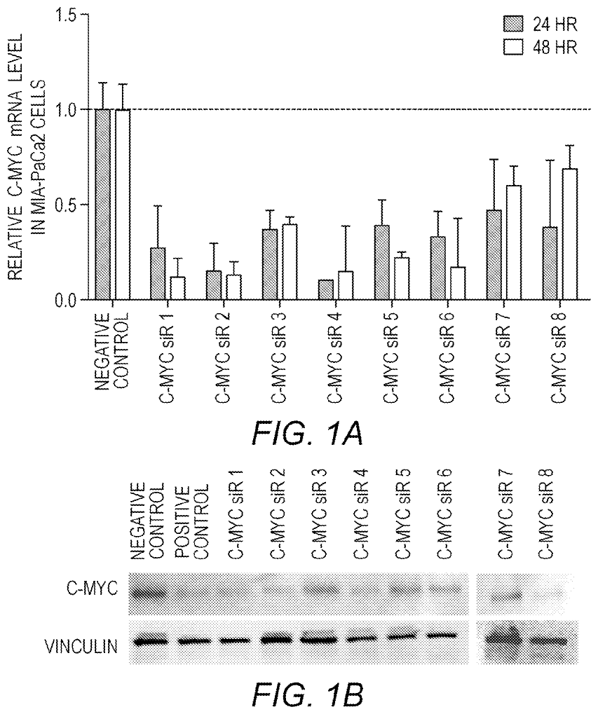

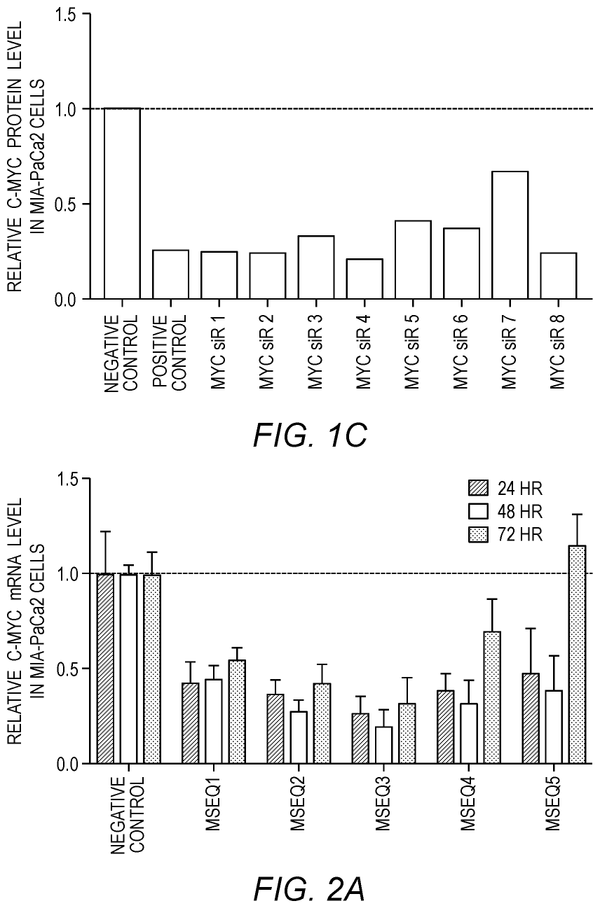

MIA-PaCa2 cells were treated with a control siRNA targeting snord90 and eight c-Myc targeting siRNAs at 20 nM for 24 and 48 hours (FIG. 1A). The control siRNA targeting snord90 and eight c-Myc targeting siRNAs are shown in Table 1. Relative quantification of c-Myc band intensity was then performed (FIG. 1C). MIA-PaCa2 cells were treated with a negative control siRNA (NC) targeting snord90, a positive control siRNA (PC) from previously published papers and eight c-Myc targeting siRNAs at 20 nM for 24 hours, then immunoblotted. Vinculin was used as an internal control (FIG. 1B). The positive control has previously been shown to suppress c-Myc expression (Vaseva et al., KRAS Suppression- Induced Degradation of MYC Is Antagonized by a MEK5-ERK5 Compensatory Mechanism. Cancer cell, 34(5), 807-822.e7, 2018). Several highly potent c-Myc siRNAs were identified as evidenced by inhibition of c-Myc mRNA and protein expression.

TABLE 1
siRNA sequences

| Oligo | S | Sense Strand Sequence (5′ to 3′) | | SEQ |
|---|---|---|---|---|
| Name | A | Antisense Strand Sequence (5′ to 3′) | Overhang | ID NO |
| cMyc1 | S | GAGGAUAUCUGGAAGAAAU | [dT][dT] | 10 |
| | A | AUUUCUUCCAGAUAUCCUC | [dT][dT] | 28 |
| cMyc2 | S | GAGAACAGUUGAAACACAA | [dT][dT] | 11 |
| | A | UUGUGUUUCAACUGUUCUC | [dT][dT] | 29 |
| cMyc3 | S | AACACAAACUUGAACAGCU | [dT][dT] | 12 |
| | A | AGCUGUUCAAGUUUGUGUU | [dT][dT] | 30 |
| cMyc4 | S | AAGAAGAUGAGGAAGAAAU | [dT][dT] | 13 |
| | A | AUUUCUUCCUCAUCUUCUU | [dT][dT] | 31 |
| cMyc5 | S | CACAGCCCACUGGUCCUCAAGA | [dT][dT] | 14 |
| | A | UCUUGAGGACCAGUGGGCUGUG | [dT][dT] | 32 |
| cMyc6 | S | AAGAGGCGAACACACAACGUC | [dT][dT] | 15 |
| | A | GACGUUGUGUGUUCGCCUCUU | [dT][dT] | 33 |
| cMyc7 | S | CAGAUCAGCAACAACCG | [dT][dT] | 16 |
| | A | CGGUUGUUGCUGAUCUG | [dT][dT] | 34 |
| cMyc8 | S | GAGACCUUCAUCAAAAACAUCAUCA | [dT][dT] | 17 |
| | A | UGAUGAUGUUUUUGAUGAAGGUCUC | [dT][dT] | 35 |
| Positive | S | GAGCUAAAACGGAGCUUUU | [dT][dT] | 18 |
| Control | A | AAAAGCUCCGUUUUAGCUC | [dT][dT] | 36 |

### EXAMPLE 2

### Synthetic Chemically Modified siRNAs Substantially Reduce c-Myc Gene Transcripts

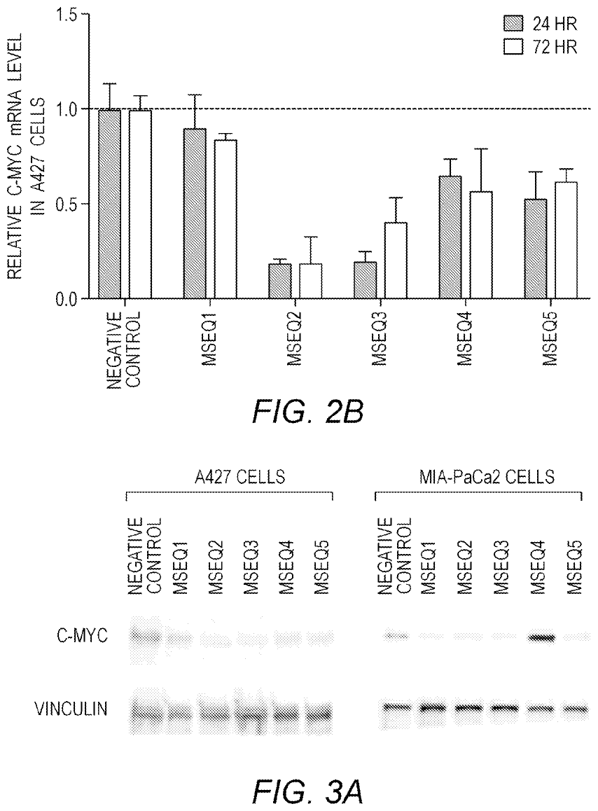

MIA-PaCa2 cells were treated with a control siRNA with 2′Ome modifications and five chemically modified c-Myc targeting siRNAs (SEQ ID NOS:37 and 38, SEQ ID NOS:39 and 40, SEQ ID NOS:41 and 42, SEQ ID NOS:43 and 44, or SEQ ID NOS:45 and 46) at 20nM for 24, 48, and 72 hours (FIG. 2A). A427 cells were treated with a control siRNA with 2 Ome modifications and five chemically modified c-Myc targeting siRNAs at 20 nM for 24 and 72 hours (FIG. 2B). These siRNAs were fully modified (FM) to minimize nuclease degradation and immune stimulation. While these types of modifications often attenuate the silencing activity of siRNAs, the inventors developed several fully modified siRNA sequences that retain all or nearly all their silencing activity compared to unmodified siRNAs. Table 2 lists the fully modified siRNA sequences that were prepared. The Hi2F pattern consists of roughly a 50/50 mixture of 2′-fluoro (2′F) and 2′-O-methyl (2′OMe) ribose modifications. The Hi2Ome pattern consists of a majority of 2 OMe ribose modifications and a minimum of 2′F modifications in order to improve in vivo stability and avoid nuclease degradation. These RT-qPCR data demonstrate that the fully modified c-Myc targeting siRNAs maintain their potent silencing activity in several cancer cell lines.

TABLE 2
Fully modified siRNA sequences

| | | | SEQ ID |
|---|---|---|---|
| Name | Strand | Sequence (5′-3′) | NO |
| cMYC | S | [2flG]*[mC]*[2flG][mA][2flG][mG][2flA][mU][2flA][2flU][2flC] | 37 |
| siRNA 1 | | [mU][2flG][mG][2flA][mA][2flG][mA][2flA][mA][2flU] | |
| Hi2F | AS | [mA]*[2flU]*[mU][2flU][mC][2flU][mU][2flC][mC][2flA][mG] | 38 |
| (Mseq1) | | [mA][mU][2flA][mU][2flC][mC][2flU][mC][2flG][mC]*[mU]*[mG] | |
| cMYC | S | [2flA]*[mC]*[2flG][mA][2flG][mA][2flA][mC][2flA][2flG][2flU] | 39 |
| SIRNA 2 | | [mU][2flG][mA][2flA][mA][2flC][mA][2flC][mA][2flA] | |
| Hi2F | AS | [mU]*[2flU]*[mG][2flU][mG][2flU][mU][2flU][mC][2flA][mA][mC] | 40 |
| (Mseq2) | | [mU][2flG][mU][2flU][mC][2flU][mC][2flG][mU]*[mC]*[mG] | |
| cMYC | S | [2flA]*[mC]*[2flA][mA][2flG][mA][2flA][mG][2flA][2flU][2flG] | 41 |
| siRNA 3 | | [mA][2flG][mG][2flA][mA][2flG][mA][2flA][mA][2flU] | |
| Hi2F | AS | [mA]*[2flU]*[mU][2flU][mC][2flU][mU][2flC][mC][2flU][mC][mA] | 42 |
| (Mseq3) | | [mU][2flC][mU][2flU][mC][2flU][mU][2flG][mU]*[mU]*[mC] | |
| cMYC | S | [2flA]*[mA]*[2flG][mA][2flG][mG][2flC][mG][2flA][2flA][2flC] | 43 |
| siRNA 4 | | [mA][2flC][mA][2flC][mA][2flA][mC][2flG][mU][2flC] | |
| Hi2F | AS | [mG]*[2flA]*[mC][2flG][mU][2flU][mG][2flU][mG][2flU][mG][mU] | 44 |
| (Mseq4) | | [mU][2flC][mG][2flC][mC][2flU][mC][2flU][mU]*[mG]*[mA] | |
| cMYC | S | [2flG]*[mA]*[2flG][mA][2flG][mC][2flU][mA][2flA][2flA][2flA] | 45 |
| siRNA 5 | | [mC][2flG][mG][2flA][mG][2flC][mU][2flU][mU][2flU] | |
| Hi2F | AS | [mA]*[2flA]*[mA][2flA][mG][2flC][mU][2flC][mC][2flG][mU][mU] | 46 |
| (Mseq5) | | [mU][2flU][mA][2flG][mC][2flU][mC][2flU][mC]*[mG]*[mU] | |
| cMYC | S | [mA]*[mC]*[mG][mA][mG][mA][2flA][mC][2flA][2flG][2flU][mU] | 51 |
| siRNA 2 | | [mG][mA][mA][mA][mC][mA][mC]*[mA]*[mA] | |
| Hi2OMe | AS | [mU]*[2flU]*[mG][mU][mG][2flU][mU][mU][mC][mA][mA][mC] | 52 |
| (Mseq2) | | [mU][2flG][mU][2flU][mC][mU][mC][mG][mU]*[mC]*[mG] | |
| cMYC | S | [mA]*[mC]*[mA][mA][mG][mA][2flA][mG][2flA][2flU][2flG][mA] | 53 |
| siRNA 3 | | [mG][mG][mA][mA][mG][mA][mA]*[mA]*[mU] | |
| Hi2OMe | AS | [mA]*[2flU]*[mU][mU][mC][2flU][mU][mC][mC][mU][mC][mA] | 54 |
| (Mseq3) | | [mU][2flC][mU][2flU][mC][mU][mU][mG][mU]*[mU]*[mC] | |
| KRAS | S | [mG]*[mU]*[mC][mU][mC][mU][2flU][mG][2flG][2flA][2flU][mA] | 47 |
| Seq2 | | [mU][mU][mC][mU][mC][mG][mA] | |
| Hi2OMe | AS | [mU]*[2flC]*[mG][mA][mG][2flA][mA][mU][mA][mU][mC][mC] | 48 |
| (Kseq2) | | [mA][2flA][mG][2flA][mG][mA][mC]*[mA]*[mG] | |
| KRAS | S | [mC]*[mA]*[mG][mC][mU][mA][2flA][mU][2flU][2flC][2flA][mG] | 49 |
| Seq3 | | [mA][mA][mU][mC][mA][mU][mU] | |
| Hi2OMe | AS | [mA]*[2flA]*[mU][mG][mA][2flU][mU][mC][mU][mG][mA][mA] | 50 |
| (Kseq3) | | [mU][2flU][mA][2flG][mC][mU][mG]*[mU]*[mA] | |
| V4-G12V | S | [mG]*[mU]*[mG][mG][mU][mA][2flG][mU][2flU][2flG][2flG][mA] | 55 |
| KRAS | | [mG][mC][mU][mG][mU][mU][mG]*[mG]*[mC] | |
| Hi2OMe | AS | [mG]*[2flC]*[mC][mA][mA][2flC][mA][mG][mC][mU][mC][mC] | 56 |
| (V4-G12V) | | [mA][2flA][mC][2flU][mA][mC][mC][mA][mC]*[mA]*[mA] | |
| V1-G12V | S | [2F1G]*[mU]*[2flA][mG][2flU][mU][2flG][mG][2flA][2flC] | 105 |
| KRAS | | [mU][2flG][mU][2flU][mG][2flG][mC][2flG][mU][2flA] | |
| Hi2F | AS | [mU]*[2flA]*[mC][2flG][mC][2flC][mA][2flA][mC][2flA][mG][mC] | |
| (V1-G12V) | | [mU][2flC][mC][2flA][mA][2flC][mU][2flA][mC]*[mC]*[mA] | 106 |
| COV1 siR | S | [mU]*[mA]*[mA][mC][mA][mA][2flU][mG][2flU][2flU][2flG][mC] | 57 |
| | | [mU][mU][mU][mU][mC][mA][mA]*[mA]*[mA] | |
| | AS | [m]*[2flU]*[mU][mU][mG][2flA][mA][mA][mA][mG][mC][mA] | 58 |
| | | [mA][2flC][mA][2flU][mU][mG][mU][mU][mA]*[mG]*[mU] | |
| COV2 siR | S | [mA]*[mC]*[mU][mG][mU][mA][2flC][mA][2flG][2flU][2flC][mU] | 59 |
| | | [mA][mA][mA][mA][mU][mG][mU]*[mC]*[mA] | |
| | AS | [mU]*[2flG]*[mA][mC][mA][2flU][mU][mU][mU][mA][mG][mA] | 60 |
| | | [mC][2flU][mG][2flU][mA][mC][mA][mG][mU]*[mG]*[mG] | |
| D2- | S | [mG]*[mU]*[mA][mG][mU][mU][mG][mG][mA][mG][mC][mU] | 61 |
| G13D- | | [mG][mG][mG][mG][mA][mC][mG][mU][mA] | |
| 20Me | AS | [mU]*[mA]*[mC][mG][mU][mC][mA][mC][mC][mA][mG][mC] | 62 |
| (Neg Ctrl) | | [mU][mC][mC][mA][mA][mC][mU][mA][mC]*[mC]*[mA] | |

S-sense strand
AS-antisense strand
m-2′-O-methyl on sugar moieties
2fl-2′-fluoro on sugar moieties
*-phosphorothioate in between nucleotides

### EXAMPLE 3

### Synthetic Chemically Modified siRNAs Decrease c-MYC Protein Expression

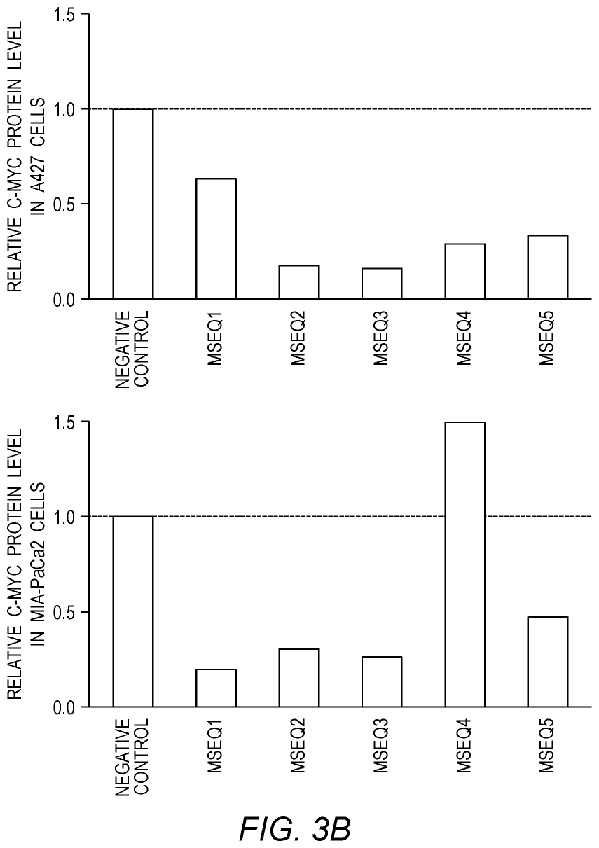

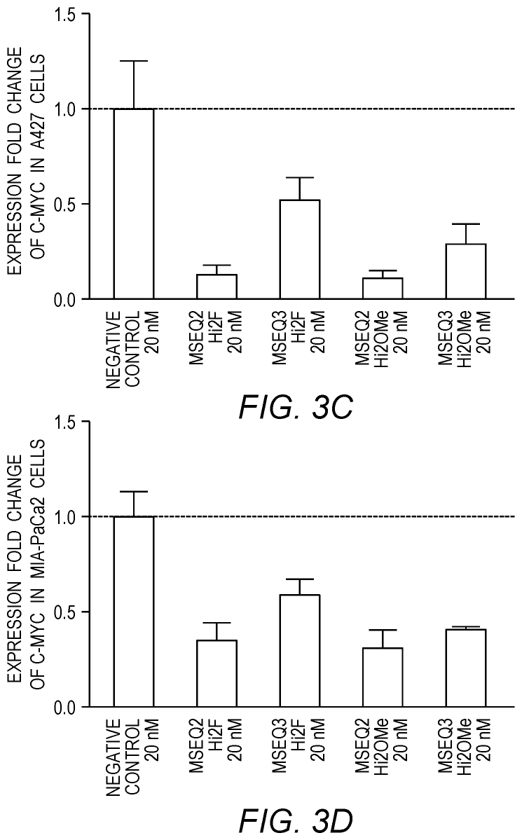

MIA-PaCa2 and A427 cells were treated with a control siRNA with 2′Ome modifications and chemically modified c-Myc targeting siRNAs (SEQ ID NOS:37 and 38, SEQ ID NOS: 39 and 40, SEQ ID NOS:41 and 42, SEQ ID NOS:43 and 44, or SEQ ID NOS:45 and 46) at 20nM for 48 hours and immunoblotted, where vinculin was used as an internal control (FIG. 3A). Relative quantification of the c-Myc band intensity was then plotted in FIG. 3B. siRNAs with two different chemical modification patterns (Hi2F and Hi2Ome (SEQ ID NOS:39 and 40, SEQ ID NOS:41 and 42, SEQ ID NOS:51 and 52, SEQ ID NOS:53 and 54)) were tested in A427 and MIA-PaCA2 cells and shown to be substantiallly equally effective (FIGS. 3C and 3D). These protein expression data demonstrate that the fully modified c-Myc targeting siRNAs maintain their potent silencing activity in several cancer cell lines.

### EXAMPLE 4

### Synthetic Chemically Modified siRNAs Decrease in In Vitro Spheroid Formation

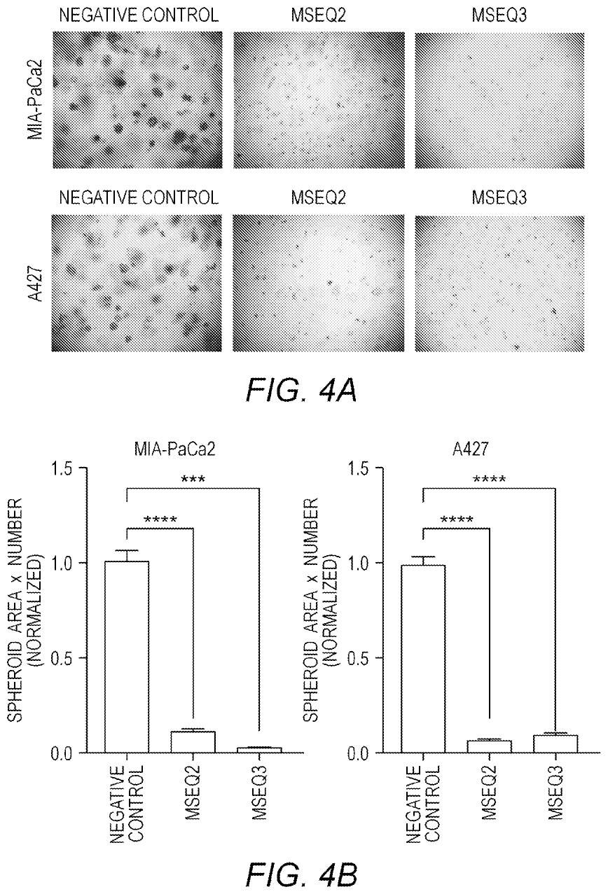

MIA-PaCa2 and A427 cells were treated with a control siRNA with 2′Ome modifications and two chemically modified c-Myc targeting siRNAs (either SEQ ID NOS:39 and 40 or SEQ ID NOS:41 and 42) at 20nM for 24 hours, then grown in Matrigel for five days. Representative phase contrast images were taken with a confocal microscope using a 5× objective (FIG. 4A). Relative quantification of the average area of spheroids from the images in FIG. 4A are shown in FIG. 4B. Statistics for the relative quantitation were: ****p=<0.0001, *** p=0.0002. These data demonstrate that the fully modified c-Myc targeting siRNAs have potent tumor growth inhibitory effects in several cancer cell lines.

### EXAMPLE 5

### Dual-targeting c-MYC and KRAS with Chemically Modified siRNAs Results in

Decreased Spheroid Area and Number in MIA-PaCa2 Cells in Matrigel

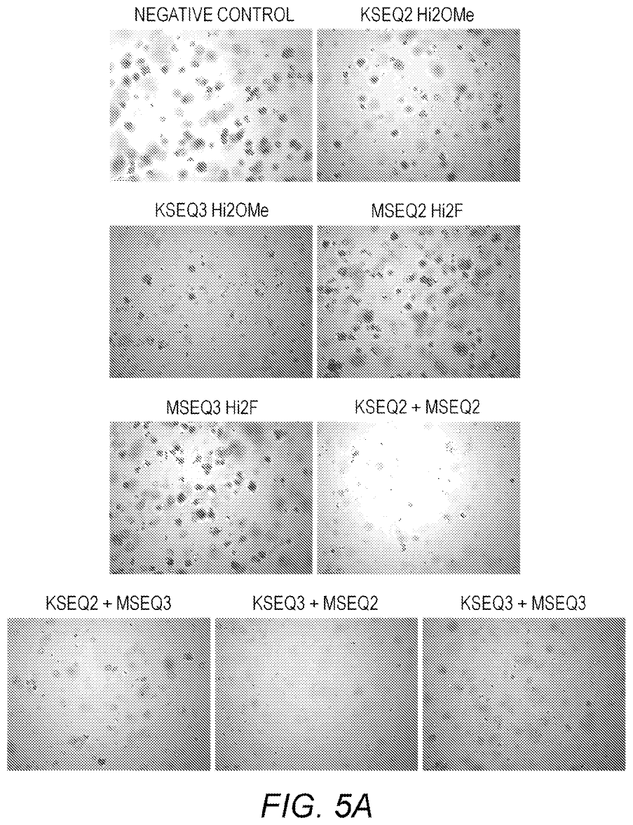

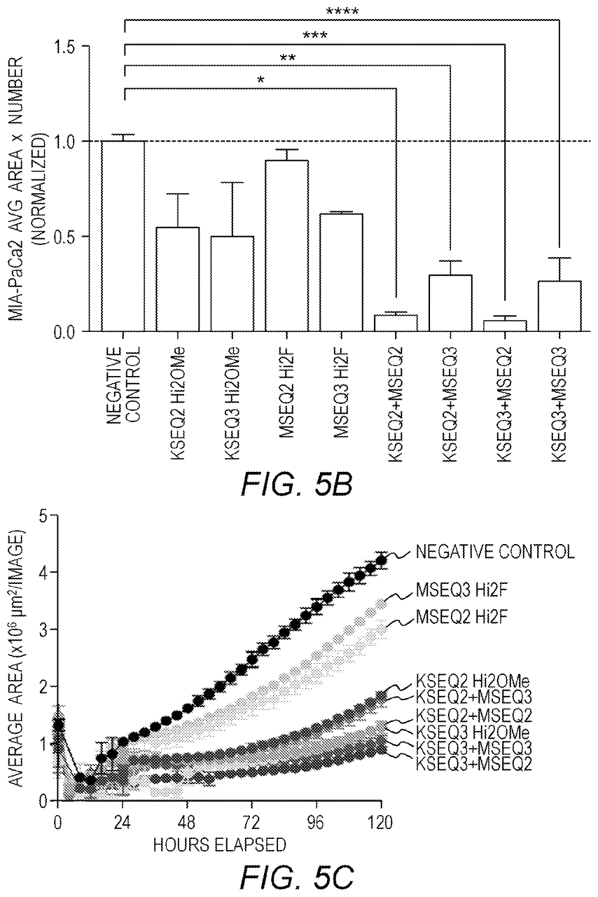

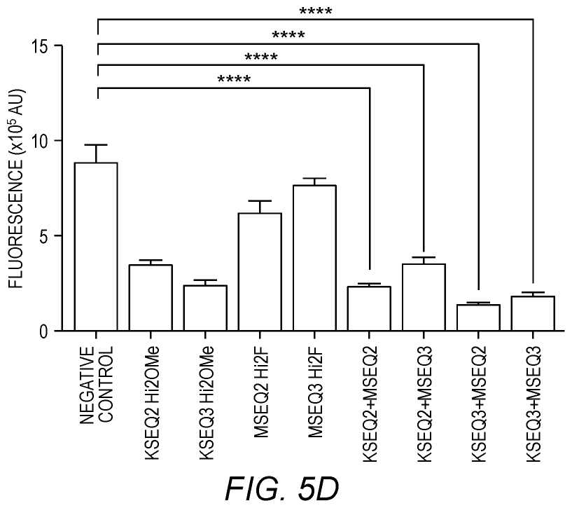

MIA-PaCa2 cells were treated with a control siRNA with 2′Ome modifications and two chemically modified c-Myc (either SEQ ID NOS:39 and 40 or SEQ ID NOS:41 and 42) and KRAS (either SEQ ID NOS:47 and 48 or SEQ ID NOS:49 and 50) targeting siRNAs at 5nM for 24 hours. then grown in Matrigel for five days. Representative phase contrast images were taken with a confocal microscope using a 5x objective (FIG. 5A). Relative quantification of spheroid area and number from confocal images was carried out using Organoseg software is shown in FIG. 5B. Statistics for the relative quantitation were: **p=0.0022 to 0.0027. *p=0.0113 to 0.0147.

MIA-PaCa2 cells were treated with a control siRNA with 2 Ome modifications and two chemically modified c-Myc (either SEQ ID NOS:39 and 40 or SEQ ID NOS:41 and 42) and KRAS (either SEQ ID NOS:47 and 48 or SEQ ID NOS:49 and 50) targeting siRNAs at 5nM for 24 hours. then seeded in a 96-well round bottom plate and imaged for five days with the Incucyte live cell analysis system (FIG. 5C). Next. post-imaging was carried out with the Incucyte Cells, which were mixed with CellTiter Glo 3D (Promega) and fluorescence was measured with a plate reader to determine metabolic activity (FIG. 5D). Thes data demonstrate that while individually targeting mutant KRAS or c-Myc by fully modified siRNAs have anti-tumor growth effects. the combination of KRAS and c-Myc siRNA molecules are even more effective. Thes data support the dual targeting of mutant KRAS and c-Myc in cancer based on inhibiting spheroid formation and proliferation within the spheroid.

### EXAMPLE 6

### Dual-targeting c-MYC and KRAS with Chemically Modified siRNAs Results in Decreased Spheroid Area and Number in A427 Cells in Matrigel

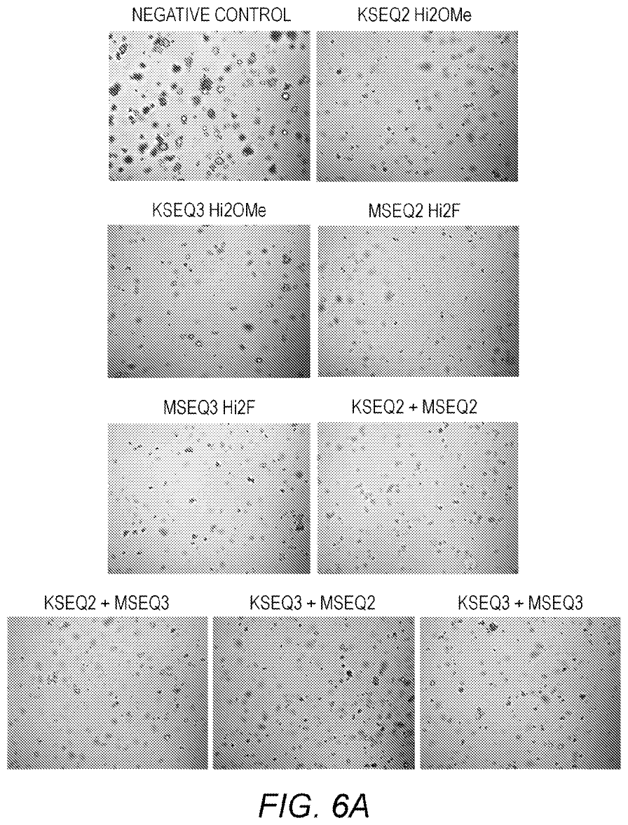

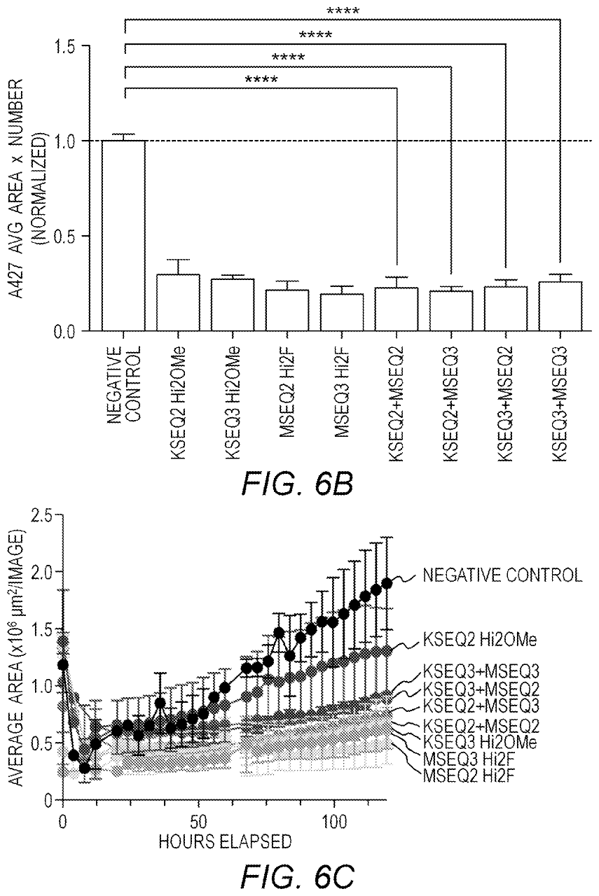

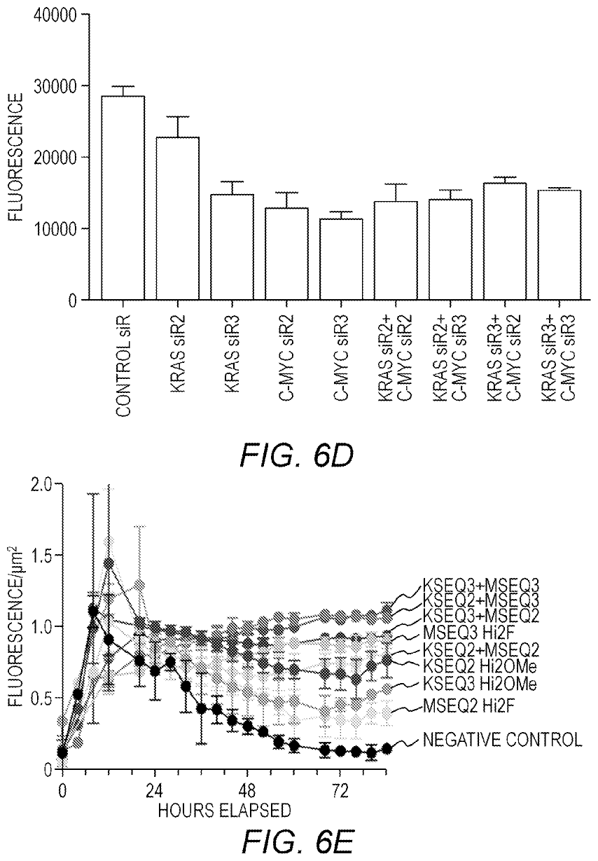

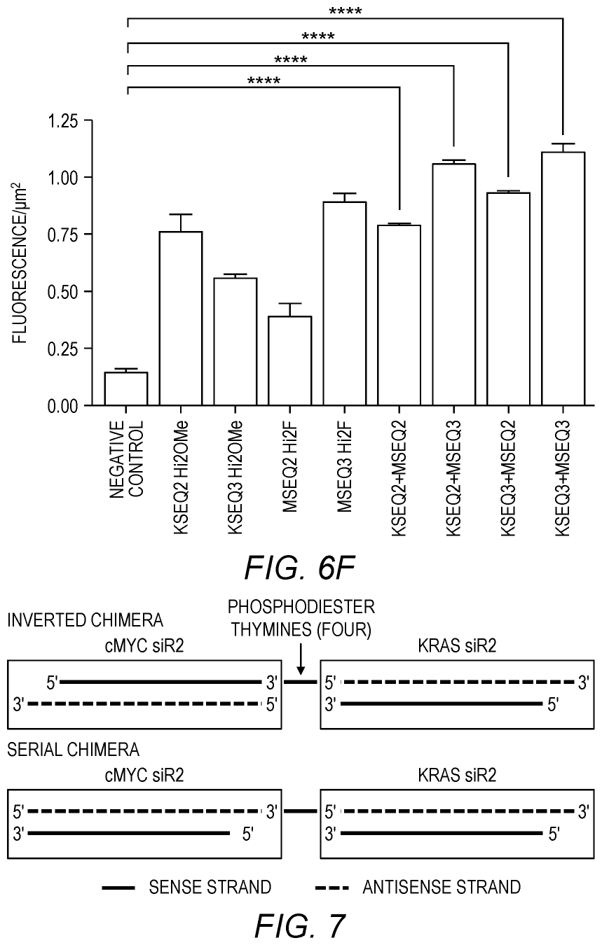

A427 cells were treated with a control siRNA with 2′Ome modifications and two chemically modified c-Myc (either SEQ ID NOS:39 and 40 or SEQ ID NOS:41 and 42) and KRAS (either SEQ ID NOS:47 and 48 or SEQ ID NOS:49 and 50) targeting siRNAs at 10 nM for 24 hours. then grown in Matrigel for five days. Representative phase contrast images were taken with a confocal microscope using a 5× objective (FIG. 6A). Relative quantification of spheroid area and number from confocal images was carried out using Organoseg software (FIG. 6B). Statistics for the relative quantitation were: ****p=<0.0001.

A427 cells were treated with a control siRNA with 2′Ome modifications and two chemically modified c-Myc (either SEQ ID NOS:39 and 40 or SEQ ID NOS:41 and 42) and KRAS (either SEQ ID NOS:47 and 48 or SEQ ID NOS:49 and 50) targeting siRNAs at 10 nM for 24 hours, then seeded in a 96-well round bottom plate and imaged for five days with the Incucyte live cell analysis system (FIG. 6C). Next. post-imaging was carried out with the Incucyte Cells, which were mixed with CellTiter Glo 3D (Promega) and fluorescence was measured with a plate reader to determine metabolic activity (FIG. 6D).

The dual-targeting of c-MYC and KRAS with chemically modified siRNAs resulted in apoptosis in A427 cells. A427 cells were treated with a control siRNA with 2′Ome modifications and two chemically modified c-Myc (either SEQ ID NOS:39 and 40 or SEQ ID NOS:41 and 42) and KRAS (either SEQ ID NOS:47 and 48 or SEQ ID NOS:49 and 50) targeting siRNAs at 10nM for 24 hours, then seeded in a 96-well round bottom plate with CellTox Green and imaged for five days with the Incucyte live cell analysis system. Graphs show data up to 84 hours (FIGS. 6E and 6F). Statistics were: ****p=<0.0001, ***p=0.0003. These data demonstrate that while individually targeting mutant KRAS or c-Myc by fully modified siRNAs have anti-tumor growth effects, the combination of KRAS and c- Myc siRNA molecules are even more effective. These data support the dual targeting of mutant KRAS and c-Myc in cancer based on induction of apoptosis within the spheroid.

### EXAMPLE 7

Phosphodiester-Linked Chemically Modified siRNAs Combining KRAS and cMyc-Targeting siRNAs (“Chimera”) is Biologically, Highly Active and Decreases c-Myc and KRAS mRNA Transcripts in MIA-PaCa2 Cells

MIA-PaCa2 cells were treated with a control siRNA with 2′Ome modifications (SEQ ID NOS:61 and 62), one chemically modified c-Myc-targeting siRNA (SEQ ID NOS:39 and 40) and one chemically modified KRAS-targeting siRNA (SEQ ID NOS:47 and 48), either alone or in combination, and one “chimera” siRNA (SEQ ID NOS:63-65) at 5 nM, 10 nM, and 20 nM for 48 or 72 hours (FIG. 8). A schematic showing the phosphodiester-linked chemically modified siR combining KRAS and cMyc-targeting siRs (“chimera”) is shown in FIG. 7, which reveals a conceptual design in which a sense strand of one siRNA is contiguously synthesized to include a phosphodiester nucleotide bridge and then an anti-sense strand of another siRNA. Then the complementary strands to each respective siRNA are duplexed to form the multivalent chimeric siRNA. The chimera can simplify the delivery process of either multiple individual siRNAs or siRNAs that target multiple targets, and also ensures equimolar targeting of both gene transcripts. This data demonstrates that multivalent chimeric siRNAs can each be functional within the cell and potently target multiple different genes at once.

TABLE 3
chimeric siRNA sequences

| | | SEQ |
|---|---|---|
| | | ID |
| Name | Sequence (5′-3′) | NO |
| M2/K2 Inverted | [2flA]*[mC]*[2flG][mA][2flG][mA][2flA][mC][2flA][2flG][2flU][mU] | 63 |
| Chimera V1 (MYC | [2flG][mA][2flA][mA][2flC][mA][2flC]*[mA]*[2flA]TTTT[mU]*[2flC]* | |
| sense-TTTT-KRAS anti- | [mG][mA][mG][2flA][mA][mU][mA][mU][mC][mC][mA][2flA][mG][2flA] | |
| sense) | [mG][mA][mC]*[mA]*[mG] | |
| cMYC siRNA 2 Hi2F | [mU]*[2flU]*[mG][2flU][mG][2flU][mU][2flU][mC][2flA][mA][mC][mU] | 64 |
| (Mseq2) anti-sense | [2flG][mU][2flU][mC][2flU][mC][2flG][mU]*[mC]*[mG] | |
| KRAS Seq2 Hi2OMe | [mG]*[mU]*[mC][mU][mC][mU][2flU][mG][2flG][2flA][2flU][mA][mU] | 65 |
| (Kseq2) sense | [mU][mC][mU][mC][mG\|mA] | |
| M2/K2 Serial Chimera | [mu]*[2flu]*[mg][2flu][mg][2flu][mu][2flu][mc][2fla][ma][mc][mu] | 66 |
| V1 (MYC anti-sense- | [2flg][mu][2flu][mc][2flu][mc][2flg][mu]*[mc]*[mg]TTTT[mU]*[2flC] | |
| TTTT-KRAS anti-sense) | *[mG][mA][mG][2flA][mA][mU][mA][mU][mC][mC][mA][2flA][mG][2flA] | |
| | [mG][mA][mC]*[mA]*[mG] | |
| cMYC siRNA 2 Hi2F | [2flA]*[mC]*[2flG][mA][2flG][mA][2flA][mC][2flA][2flG][2flU][mU] | 67 |
| (Mseq2) sense | [2flG][mA][2flA][mA][2flC][mA][2flC]*[mA]*[2flA] | |
| KRAS Seq2 Hi2OMe | [mG]*[mU]*[mC][mU][mC][mU][2flU][mG][2flG][2flA][2flU][mA][mU] | 68 |
| (Kseq2) sense | [mU][mC][mU][mC]*[mG]*[mA] | |
| M2/K2 Inverted | [mA]*[mC]*[mG][mA][mG][mA][2flA][mC][2flA][2flG][2flU][mU][mG] | 69 |
| Chimera V2 (MYC | [mA][mA][mA][mC][mA][mC]*[mA]*[mA]TTTT[mU]*[2flC]*[mG][mA] | |
| sense-TTTT-KRAS anti- | [mG][2flA][mA][mU][mA][mU][mC][mC][mA][2flA][mG][2flA][mG][mA] | |
| sense) | [mC]*[mA]*[mG] | |
| cMYC siRNA 2 | [mU]*[2flU]*[mG][mU][mG][2flU][mU][mU][mC][mA][mA][mC][mU] | 70 |
| Hi2OMe (Mseq2) anti- | [2flG][mU][2flU][mC][mU][mC][mG][mU]*[mC]*[mG] | |
| sense | | |
| KRAS Seq2 Hi2OMe | [mG]*[mU]*[mC][mU][mC][mU][2flU][mG][2flG][2flA][2flU][mA][mU] | 71 |
| (Kseq2) sense | [mU][mC][mU][mC]*[mG]*[mA] | |
| K2/M2 Inverted | [mG]*[mU]*[mC][mU][mC][mU][2flU][mG][2flG][2flA][2flU][mA][mU] | 72 |
| Chimera V2 (KRAS | [mU][mC][mU][mC]*[mG]*[mA]TTTT[mU]*[2flU]*[mG][mU][mG][2flU] | |
| sense-TTTT-MYC anti- | [mU][mU][mC][mA][mA][mC][mU][2flG][mU][2flU][mC][mU][mC][mG] | |
| sense) | [mU]*[mC]*[mG] | |
| KRAS Seq2 Hi2OMe | [mU]*[2flC]*[mG][mA][mG][2flA][mA][mU][mA][mU][mC][mC][mA] | 73 |
| (Kseq2) anti-sense | [2flA][mG\|2flA][mG][mA][mC]*[mA]*[mG] | |
| cMYC siRNA 2 | [mA]*[mC]*[mG][mA][mG][mA][2flA][mC][2flA][2flG][2flU][mU][mG] | 74 |
| Hi2OMe (Mseq2) sense | [mA][mA][mA][mC][mA][mC]*[mA]*[mA] | |
| M2/V4-G12V Inverted | [mA]*[mC]*[mG][mA][mG][mA][2flA][mC][2flA][2flG][2flU][mU][mG] | 75 |
| Chimera (MYC sense- | [mA][mA][mA][mC][mA][mA]*[mA]*[mA]TTTT[mG]*[2flC]*[mC][mA] | |
| TTTT-KRAS (G12V- | [mA][2flC][mA][mG][mC][mU][mC][mC][mA][2flA][mC][2flU][mA][mC] | |
| specific) anti-sense) | [mC][mA][mC]*[mA]*[mA] | |
| cMYC siRNA 2 | [mU]*[2flU]*[mG][mU][mG][2flU][mU][mU][mC][mA][mA][mC][mU] | 76 |
| Hi2OMe (Mseq2) anti- | [2flG][mU][2flU][mC][mU][mC][mG][mU]*[mC]*[mG] | |
| sense | | |
| V4-G12V Hi2OMe (V4- | [mG]*[mU]*[mG][mG][mU][mA][2flG][mU][2flU][2flG][2flG][mA][mG] | 77 |
| G12V) sense | [mC][mU][mG][mU][mU][mG]*[mG]*[mC] | |
| M2/D2-G13D Inverted | [mA]*[mC]*[mG][mA][mG][mA][2flA][mC][2flA][2flG][2flU][mU][mG] | 78 |
| Chimera (MYC sense- | [mA][mA][mA][mC][mA][mC]*[mA]*[mA]TTTT[mU]*[2flA]*[mC][mG] | |
| TTTT-KRAS (G13D- | [mG][2flC][mA][mC][mC][mA][mG][mC][mU][2flC][mC][2flA][mA][mA] | |
| specific) anti-sense) | [mU]*[mA]*[mC] | |
| cMYC siRNA 2 | [mU]*[2flU]*[mG][mU][mG][2flU][mU][mU][mC][mA][mA][mC][mU] | 79 |
| Hi2OMe (Mseq2) anti- | [2flG][mU][2flU][mC][mU][mC][mG][mU]*[mC]*[mG] | |
| sense | | |
| D2-G13D Hi2OMe (D2- | [mA]*[mG]*[mU][mU][mG][mG][2flA][mG][2flC][2flU][2flG][mG][mU] | 80 |
| G13D) sense | [mG][mA][mC][mG]*[mU]*[mA] | |
| K3/K2 Inverted Chimera | [mC]*[mA]*[mG][mC][mU][mA][2flA][mU][2flU][2flC][2flA][mG][mA] | 81 |
| (KRAS seq3 sense- | [mA][mU][mC][mA]*[mU]*[mU]TTTT[mU]*[2flC]*[mG][mA][mG][2flA] | |
| TTTT-KRAS seq2 anti- | [mA][mU][mA][mU][mC][mC][mA][2flA][mG][2flA][mG][mA][mC]* | |
| sense) | [mA]*[mG] | |
| KRAS Seq3 Hi2OMe | [mA]*[2flA]*[mU][mG][mA][2flU][mU][mC][mU][mG][mA][mA][mU] | 82 |
| (Kseq3) anti-sense | [2flU][mA][2flG][mC][mU][mG]*[mU]*[mA] | |
| KRAS Seq2 Hi2OMe | [mG]*[mU]*[mC][mU][mC][mU][2flU][mG][2flG][2flA][2flU][mA][mU] | 83 |
| (Kseq2) sense | [mU][mC][mU][mC]*[mG]*[mA] | |
| M2/M3 Inverted | [mA]*[mC]*[mG][mA][mG][mA][2flA][mC][2flA][2flG][2flU][mU][mG] | 84 |
| Chimera (MYC seq2 | [mA][mA][mA][mC][mA][mC]*[mA]*[mA]TTTT[mA]*[2flU]*[mU][mU] | |
| sense-TTTT-MYC seq3 | [mC][2flU][mU][mC][mC][mU][mC][mA][mU][2flC][mU][2flU][mC][mU] | |
| anti-sense) | [mU][mG][mU]*[mU]*[mC] | |
| cMYC siRNA 2 | [mU]*[2flU]*[mG][mU][mG][2flU][mU][mU][mC][mA][mA][mC][mU] | 85 |
| Hi2OMe (Mseq2) anti- | [2flG][mU][2flU][mC][mU][mC][mG][mU]*[mC]*[mG] | |
| sense | | |
| cMYC siRNA 3 | [mA]*[mC]*[mA][mA][mG][mA][2flA][mG][2flA][2fU][2flG][mA][mG] | 86 |
| Hi2OMe (Mseq3) sense | [mG][mA][mA][mG][mA][mA]*[mA]*[mU] | |
| M3/M2 Inverted | [mA]*[mC]*[mA][mA][mG][mA][2flA][mG][2flA][2flU][2flG][mA][mG] | 87 |
| Chimera (MYC seq3 | [mG][mA][mA][mG][mA][mA]*[mA]*[mU]TTTT[mU]*[2flU]*[mG][mU] | |
| sense-TTTT-MYC seq2 | [mG][2flU][mU][mU][mC][mA][mA][mC][mU][2flG][mU][2flU][mC][mU] | |
| anti-sense) | [mC\|[mG][mU]*[mC]*[mG] | |
| cMYC siRNA 3 | [mA]*[2flU]*[mU][mU][mC][2flU][mU][mC][mC][mU][mC][mA][mU] | 88 |
| Hi2OMe (Mseq3) anti- | [2flC][mU][2flU][mC][mU][mU][mG][mU]*[mU]*[mC] | |
| sense | | |
| cMYC siRNA 2 | [mA]*[mC]*[mG][mA][mG][mA][2flA][mC][2flA][2flG][2flU][mU][mG] | 89 |
| Hi2OMe (Mseq2) sense | [mA][mA][mA][mC][mA][mC]*[mA]*[mA] | |
| M2/IRF4-10 (MYC seq2 | [mA]*[mC]*[mG][mA][mG][mA][2flA][mC][2flA][2flG][2flU][mU][mG] | 90 |
| sense-TTTT-IRF4 seq10 | [mA][mA][mA][mC][mA][mC]*[mA]*[mA]TTTT[mA]*[2flU]*[mU][mA] | |
| anti-sense) | [mC][2flU][mU][mA][mG][mA][mC][mA][mU][2flU][mA][2flC][mU][mA] | |
| | [mG][mA][mU]*[mU]*[mG] | |
| cMYC siRNA 2 | [mU]*[2flU]*[mG][mU][mG][2flU][mU][mU][mC][mA][mA][mC][mU] | 91 |
| Hi2OMe (Mseq2) anti- | [2flG][mU][2flU][mC][mU][mC][mG][mU]*[mC]*[mG] | |
| sense | | |
| IRF4 siRNA 10 Hi2OMe | [mA]*[mU]*[mC][mU][mA][mG][2flU][mA][2flA][2flU][2flG][mU][mC] | 92 |
| (IRseq10) sense | [mU][mA][mA][mG][mU][mA]*[mA]*[mU] | |
| Inv COV1-2 (COV1 siR | [mU]*[mA]*[mA][mC][mA][mA][2flU][mG][2flU][2flU][2flG][mC][mU] | 93 |
| sense-TTTT-COV2 siR | [mU][mU][mU][mC][mA][mA]*[mA]*[mA]TTTT[mU]*[2flG]*[mA][mC] | |
| anti-sense) | [mA][2flU][mU][mU][mU][mA][mG][mA][mC][2flU][mG][2flU][mA][mC] | |
| | [mA][mG][mU]*[mG]*[mG] | |
| COV1 siR anti-sense | [mU]*[2flU]*[mU][mU][mG][2flA][mA][mA][mA][mG][mC][mA][mA] | 94 |
| | [2flC][mA][2flU][mU][mG][mU][mU][mA]*[mG]*[mU] | |
| COV2 siR sense | [mA]*[mC]*[mU][mG][mU][mA][2flC][mA][2flG][2flU][2flC][mU][mA] | 95 |
| | [mA[mA][mA][mU][mG][mU]*[mC]*[mA] | |
| Serial COV1-2 (COV1 | [mU]*[2flU]*[mU][mU][mG][2flA][mA][mA][mA][mG][mC][mA][mA] | 96 |
| siR anti-sense-TTTT- | [2flc][mA][2flU][mU][mG][mU][mU][mA]*[mG]*[mU]TTTT[mU]*[2flG]* | |
| COV2 siR anti-sense) | [mA][mC][mA][2flU][mU][mU][mU][mA][mG][mA][mC][2flU][mG][2flU] | |
| | [mA]\|mC\|mA][mG]\|mU]*[mG]*[mG] | |
| COV1 siR sense | [mU]*[mA]*[mA][mC][mA][mA][2flU][mG][2flU][2flU][2flG][mC][mU] | 97 |
| | [mU][mU][mU][mC][mA][mA]*[mA]*[mA] | |
| COV2 siR sense | [mA]*[mC]*[mU][mG][mU][mA][2flC][mA][2flG][2flU][2flC][mU][mA] | 98 |
| | [mA[mA][mA][mU][mG][mU]*[mC]*[mA] | |
| Inv COV2-1 (COV2 siR | [mA]*[mC*[mU][mG][mU][mA][2flC][mA][2flG][2flU][2flC][mU][mA] | 99 |
| sense-TTTT-COV1 siR | [mA][mA][mA][mU][mG][mU]*[mC]*[mA]TTTT[mU]*[2flU]*[mU][mU] | |
| anti-sense) | [mG][2flA][mA][mA][mA][mG][mC][mA][mA][2flC][mA][2flU][mU][mG] | |
| | [mU][mU][mA]*[mG]*[mU] | |
| COV2 siR anti-sense | [mU]*[2flG]*[mA][mC][mA][2flU][mU][mU][mU][mA][mG][mA][mC] | 100 |
| | [2flU][mG][2flU][mA][mC][mA][mG][mU]*[mG]*[mG] | |
| COV1 siR sense | [mU]*[mA]*[mA][mC][mA][mA][2flU][mG][2flU][2flU][2flG][mC][mU] | 101 |
| | [mU][mU][mU][mC][mA][mA]*[mA]*[mA] | |
| Serial COV2-1 (COV2 | [mU]*[2flG]*[mA][mC][mA][2flU][mU][mU][mU][mA][mG][mA][mC] | 102 |
| siR anti-sense-TTTT- | [2flU][mG][2flU][mA][mC][mA][mG][mU]*[mG]*[mG]TTTT[mU]*[2fUu] | |
| COV1 siR anti-sense) | *[mU][mU][mG][2flA][mA][mA][mA][mG][mC][mA][mA][2flC][mA] | |
| | [2flU][mU][mG\|mU][mU][mA]*[mG]*[mU] | |
| COV2 siR sense | [mA]*[mC]*[mU][mG][mU][mA][2flC][mA][2flG][2f]U][2flC][mU][mA] | 103 |
| | [mA][mA][mA][mU][mG][mU]*[mC]*[mA] | |
| COV1 siR sense | [mU]*[mA]*[mA][mC][mA][mA][2flU][mG][2flU][2flU][2flG][mC][mU] | 104 |
| | [mU][mU][mU][mC][mA][mA]*[mA]*[mA] | |

### EXAMPLE 8

### Inverted Chimera is more Potent at Decreasing c-Myc and KRAS mRNA Transcripts and Protein Levels than Serial Chimera in A427 Cells

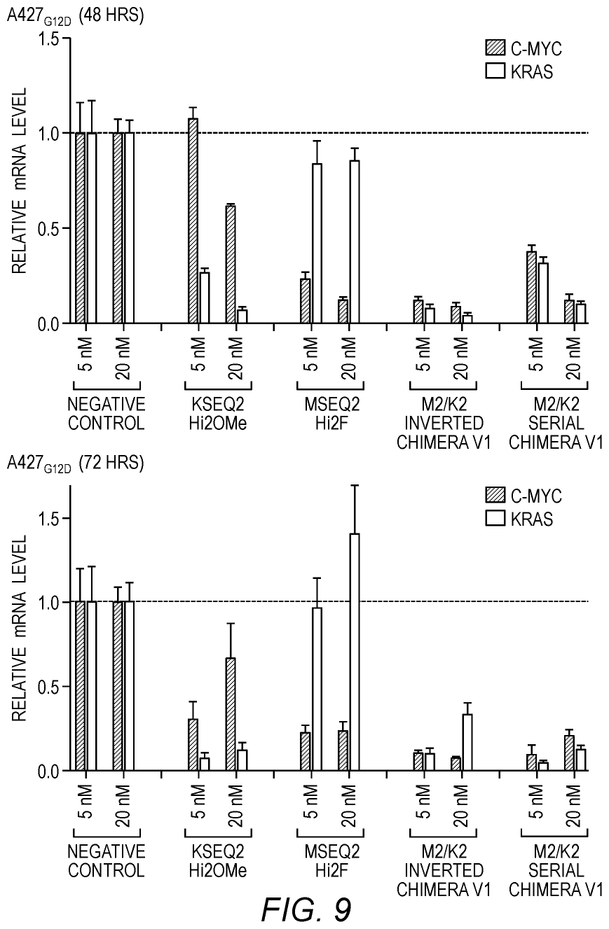

A427 cells were treated with a control siRNA with 2′Ome modifications (SEQ ID NOS:61 and 62), one chemically modified (Hi2F) c-Myc-targeting siRNA (SEQ ID NOS:39 and 40), one chemically modified (Hi2OMe) KRAS-targeting siRNA (SEQ ID NOS:47 and 48), one M2/K2 “inverted chimera” (SEQ ID NOS:63-65), or one M2/K2 “serial chimera” siRNA (SEQ ID NOS:66-68) at 5nM and 20nM for 48 and 72 hrs (FIG. 9).

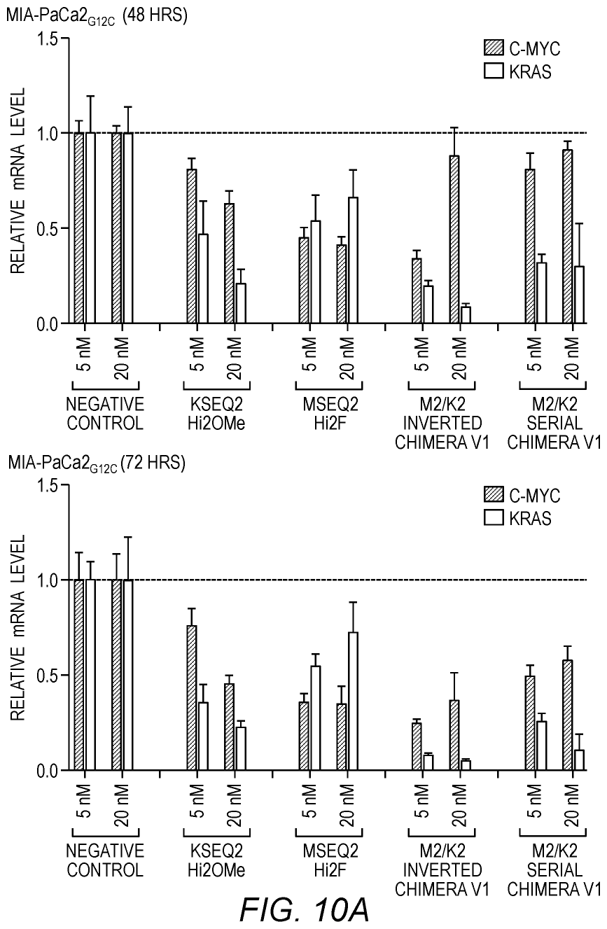

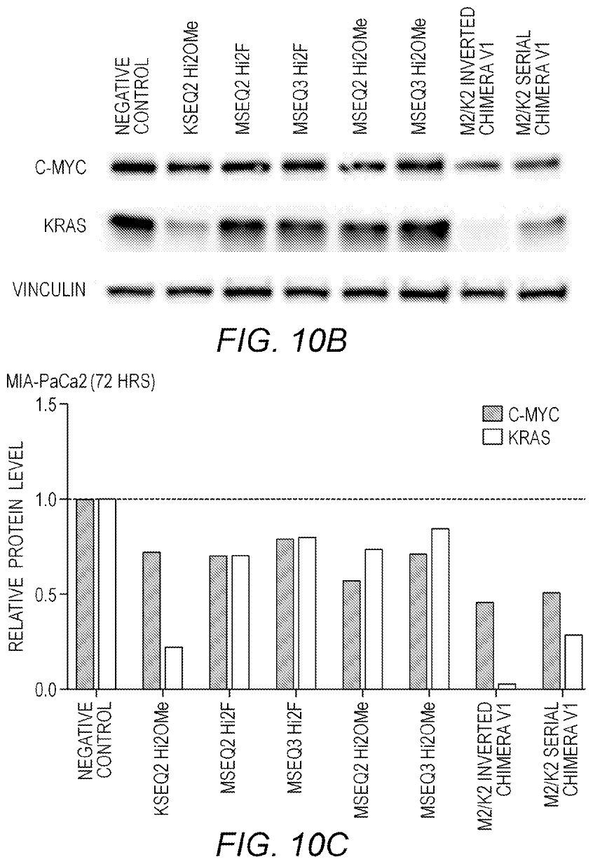

MIA-PaCa2 cells were treated with a control siRNA with 2′Ome modifications (SEQ ID NOS:61 and 62), one or two chemically modified (Hi2F) c-Myc-targeting siRNAs (SEQ ID NOS:39 and 40, SEQ ID NOS:41 and 42, SEQ ID NOS:51 and 52 SEQ ID NOS:53 and 54), one chemically modified (Hi2OMe) KRAS-targeting siRNA (SEQ ID NOS:47 and 48), one M2/K2 “inverted chimera” (SEQ ID NOS:63-65), or one M2/K2 “serial chimera” siRNA (SEQ ID NOS:66-68) at 5 nM and 20 nM for 48 and 72 hrs (FIGS. 10A-10C). Together, these data demonstrate in 2 different cancer types (A427=lung adenocarcinoma, MIA-PaCa2=pancreatic adenocarcinoma) with unique KRAS mutations, that the inverted chimeras are unexpectedly more potent than the serial chimeric designs, and that the potency of the inverted designs exceeds that of the individual siRNAs (e.g. KRAS=KSeq2 or Myc =MSeq2). These data demonstrate the potency of the inverted designs is true at the transcript (RT-qPCR) and protein (Western Blot) level.

### EXAMPLE 9

### Stability of Inverted and Serial Chimeras

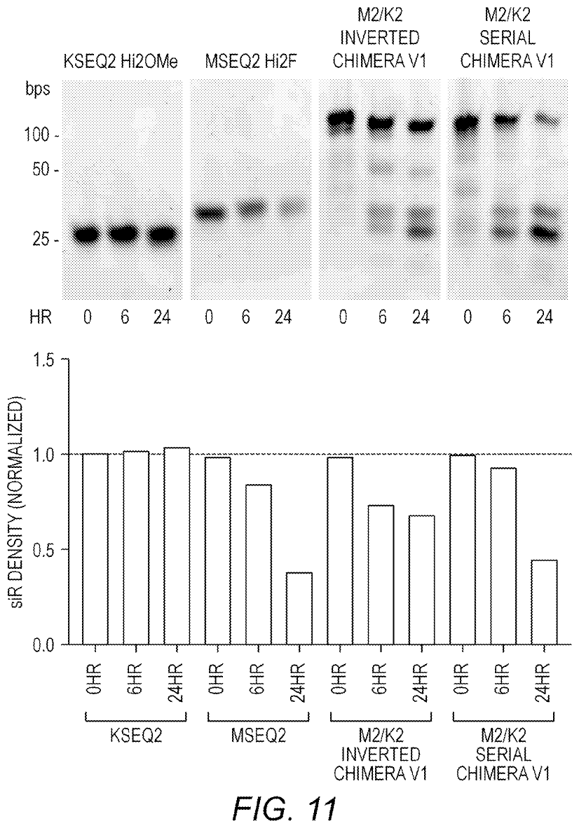

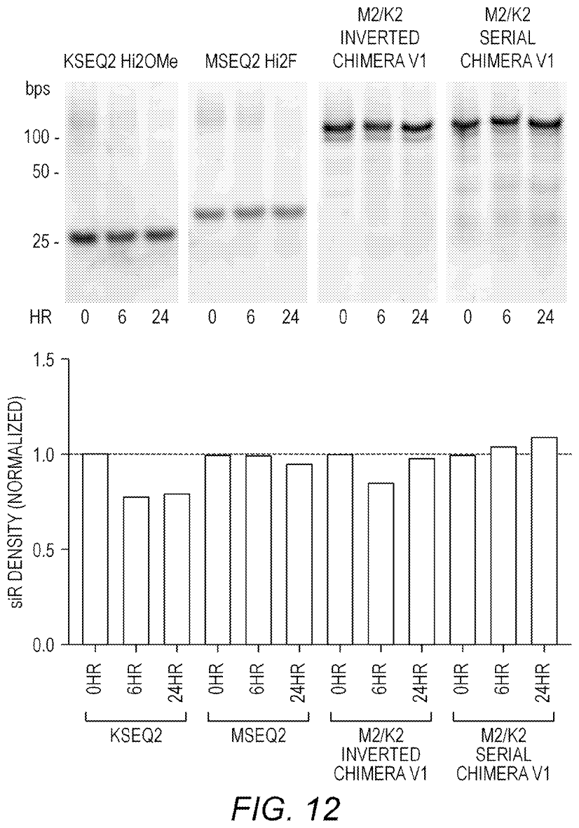

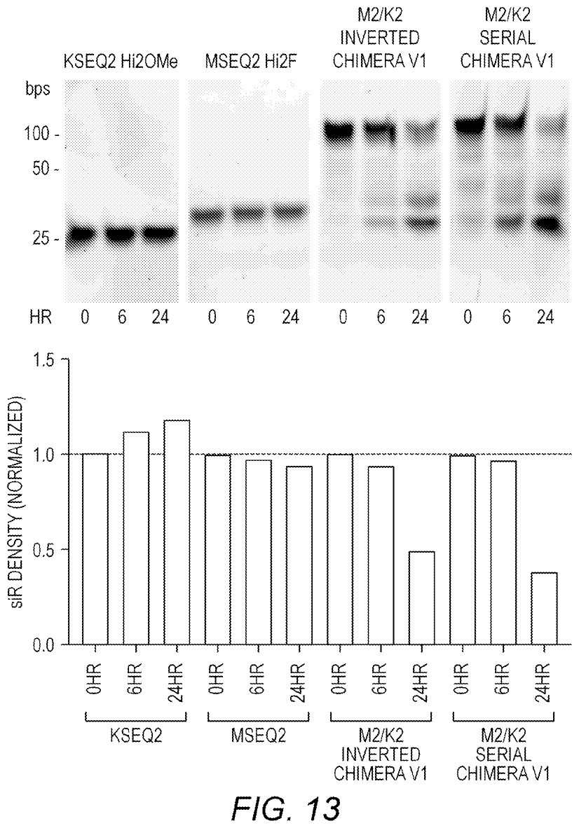

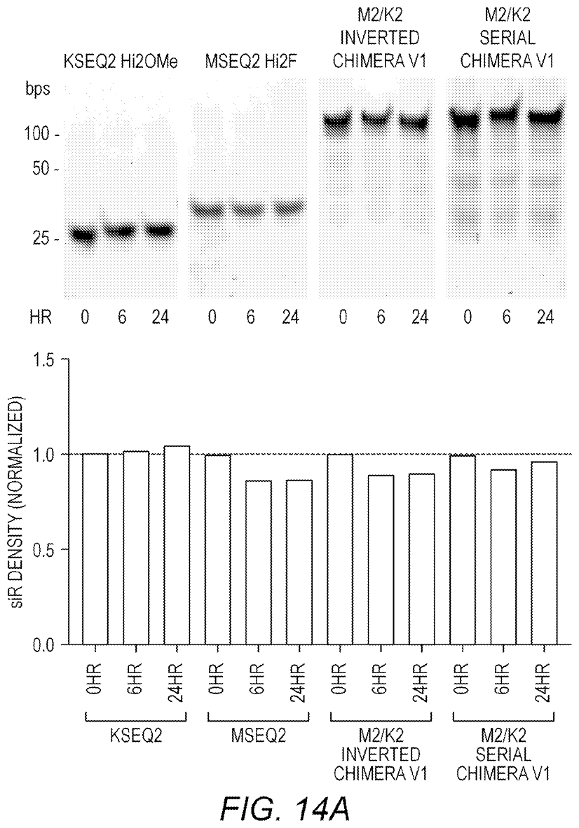

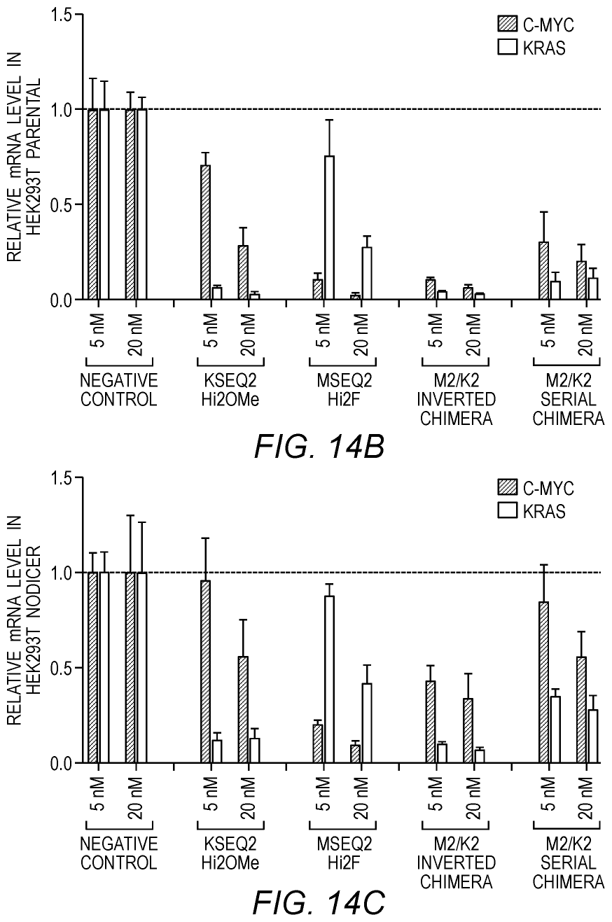

The stability of inverted and serial chimeras in serum was tested (FIG. 11). Inverted chimera was shown to be more stable. In contrast, inverted and serial chimeras were both stable in cytosolic conditions (FIG. 12). When tested in endosomal conditions, both inverted and serial chimeras began to dissociate over time (FIG. 13). Both inverted and serial chimeras were demonstrated to be resistant to cleavage by Dicer (FIGS. 14A-14C). These data demonstrate that the metabolism of the inverted and serial chimeras predominately occurs within the endosomal compartment, and very little degradation takes place in the serum conditions. Additionally, the chimeras are very stable in the cytosol and are not substrates for cleavage by Dicer. Based on this information, it is expected that these chimeric molecules will be stable in circulation, however upon receptor-mediated endocytosis, they will be cleaved at the DNA bridge. The data indicate that approximately half of the chimeras are cleaved in the endosomal compartment by approximately 24 hours after entry.

### EXAMPLE 10

### Increased Potency of Inverted Chimeras

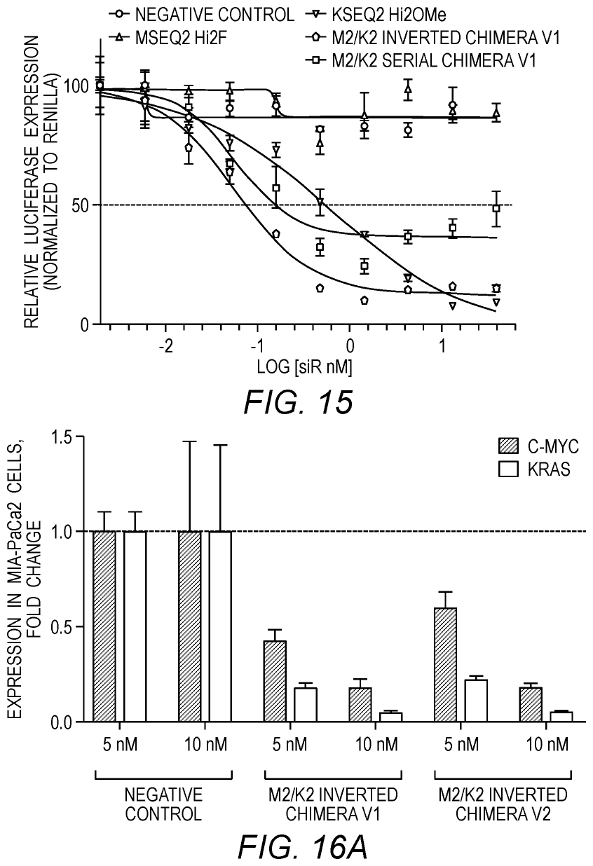

An A431 isogenic cell line that has endogenous KRAS removed by CRISPR/Cas9, and stably integrates KRAS-Firefly luciferase and untagged Renilla luciferase was treated with a control siRNA with 2′Ome modifications, one chemically modified (Hi2F) c-Myc-targeting siRNA, one chemically modified (Hi2OMe) KRAS-targeting siRNA, one M2/K2 “inverted chimera”, or one M2/K2 “serial chimera” siRNA for 96 hrs at doses ranging from 39 to 0.002 nM. Firefly luciferase data was normalized with Renilla luciferase (FIG. 15). The data show that the inverted chimera was unexpectedly far more potent than the serial chimera, as well as the individualKRAS siRNA (KSeq2 Hi2OMe).

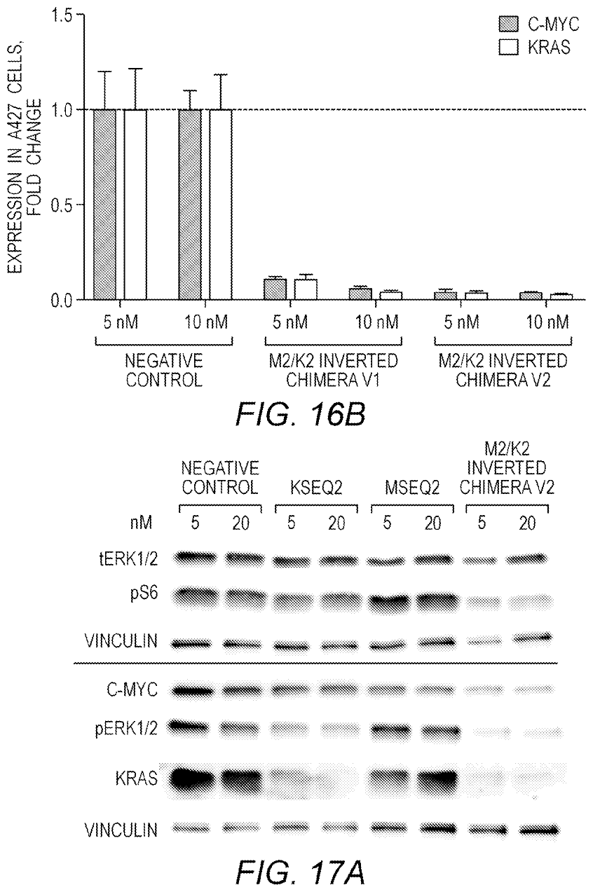

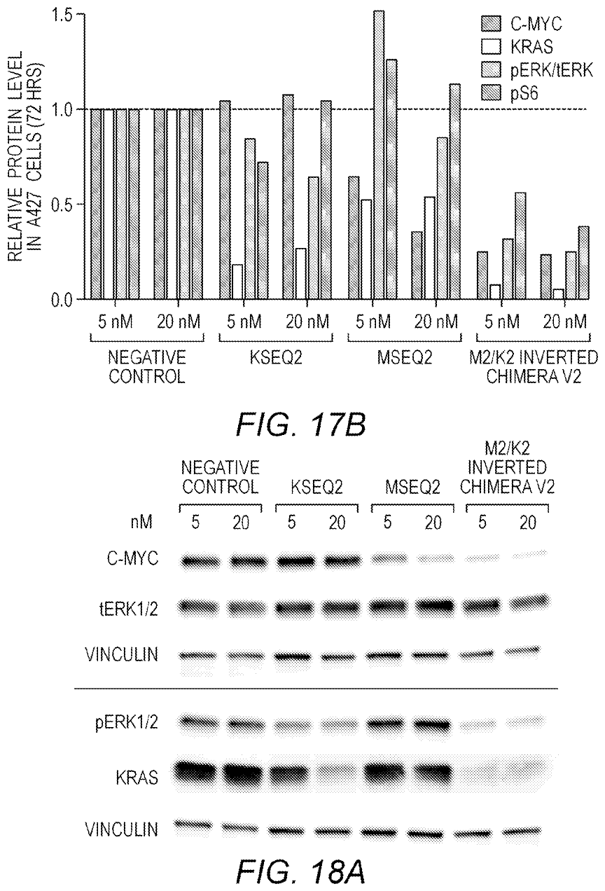

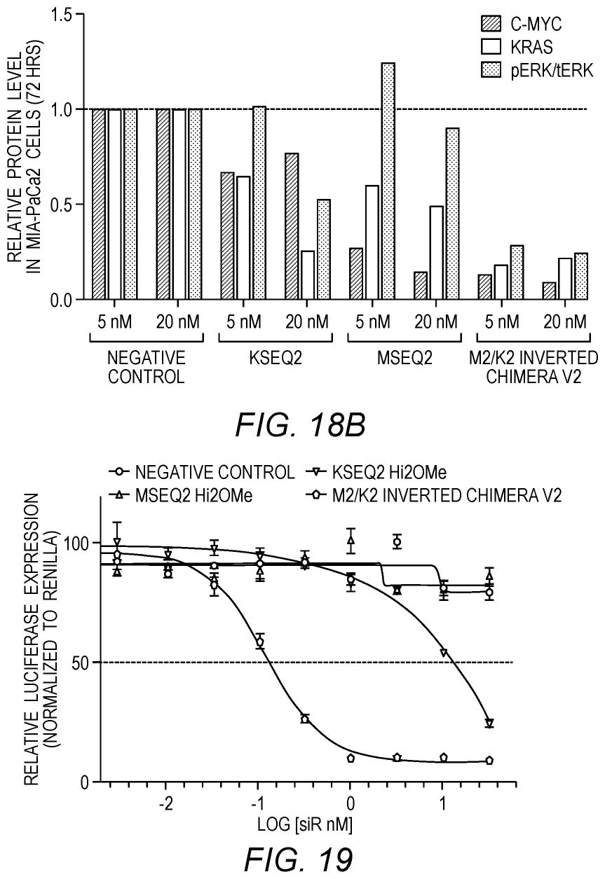

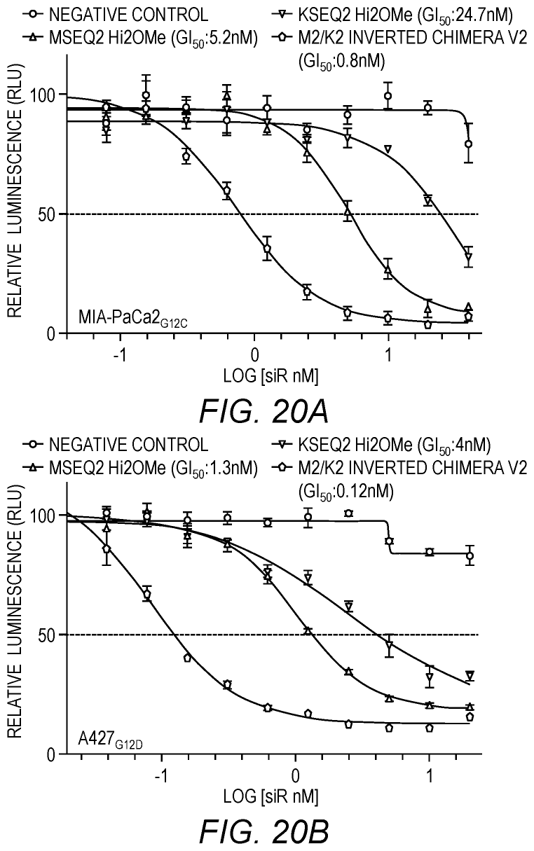

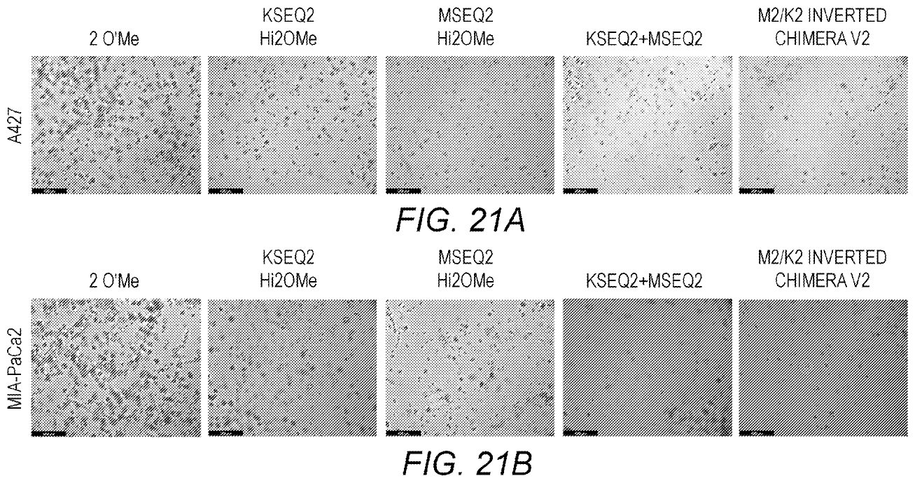

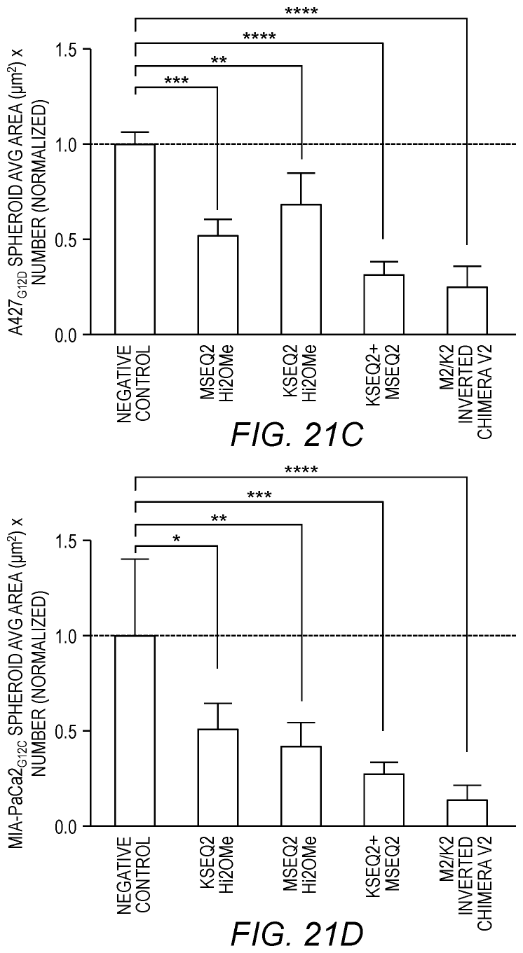

Two different versions of a c-Myc/KRAS inverted chimera (VI (SEQ ID NOS:63- 65) and V2 (SEQ ID NOS:69-71)) were equally potent against MIA-PaCA2 and A427 cells (FIGS. 16A-16B). These Hi20Me (V2) designed chimeras are expected to be more stable in vivo due to increased resistance to nuclease degradation. These data demonstrate that the V2 version of the inverted chimera is more effective than single siRNAs at reducing c-Myc and KRAS protein in A427 cells (FIGS. 17A-17B) and MIA-PaCa2 cells (FIGS. 18A-18B). These data demonstrate that downstream MAP kinase signaling and MYC protein expression are substantially attenuated by the inverted chimeric designs, and are far superior to the individual contributions seen by the individual siRNA components. Additionally, the V2 inverted chimera showed unexpectedly high potency against KRAS-luciferase protein compared to single siRNAs (FIG. 19), resulting in a substantial leftward shift in potency. Further, the V2 inverted chimera synergistically decreased cell viability in both MIA-PaCA2 and A427 cells (FIGS. 20A-20B), demonstrating an approximate 10-fold improvement in potency compared with the individual siRNAs. The V2 inverted chimera was able to decrease spheroid viaiblity in both MIA-PaCA2 and A427 cells (FIGS. 21A-21D). These data demonstrate that more optimized (V2) designs are highly effective at co-targeting 2 unique targets at once, and are surprisingly more effective than the individual siRNA components of the chimeric designs, and also consistently outperform the potency of the serial chimeric designs. The improvements in potency and ability to co-target 2 different genes results in marked improvements in inhibitory activity, at times greater than a 10-fold improvement in potency compared with the individual siRNAs.

### EXAMPLE 11

### Development and Validation of Specific KRAS Mutation (G12V) siRNAs Using Isogenic Cell Lines Expressing KRAS WT or KRAS G12V Reporters

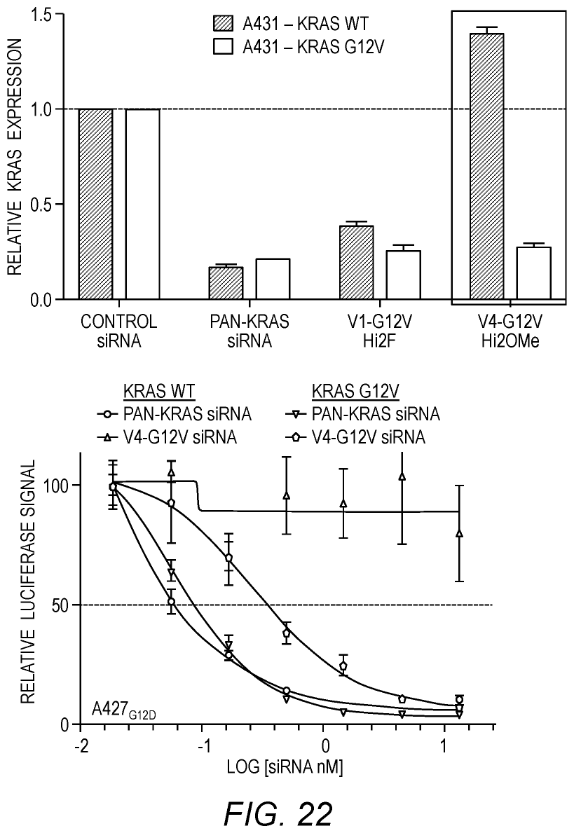

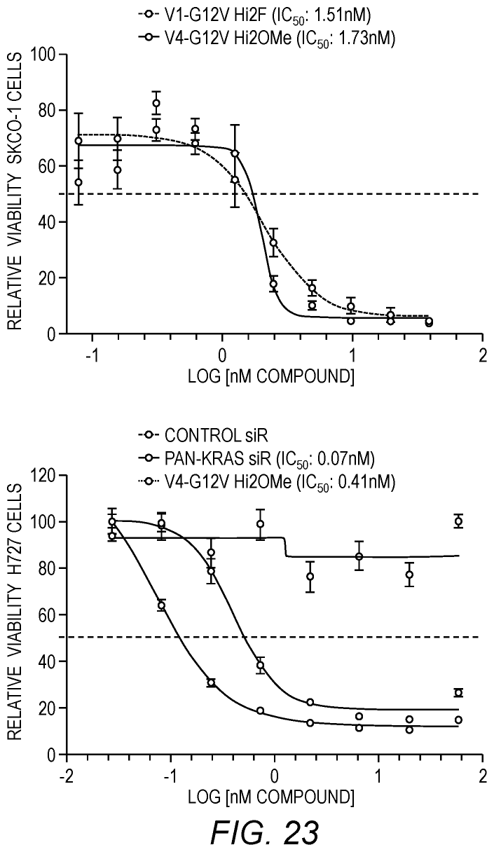

To test the specificity of KRAS siRNAs, the siRNAs were tested against A431 isogenic cells expressing wild-type KRAS or G12V KRAS (FIG. 22). The VI-G12V Hi2F or V4-G12V Hi20Me siRNAs provided potent silencing of KRAS G12V but either partially (V1-G12V) or fully (V4-G12V) spared KRAS WT targeting based on RT-qPCR. Compared to a pan-KRAS siRNA, using a luciferase readout, it was found that V4-G12V Hi20Me potently targets KRAS G12V but fully spares targeting of KRAS WT on a protein level. Similarly, potent anti-proliferative effects were seen with SKCOI (colon) and H727 (lung) cell lines with KRAS G12V mutations using a viability assay (FIG. 23). These data demonstrate that both V1-G12V and V4-G12V are capable of sparing KRAS WT while potently targeting KRAS G12V. As a result, the data demonstrate both of these compositions have potent anti-proliferative effects on KRAS G12V mutant cancer cell lines.

### EXAMPLE 12

### Potency of Inverted Chimeras

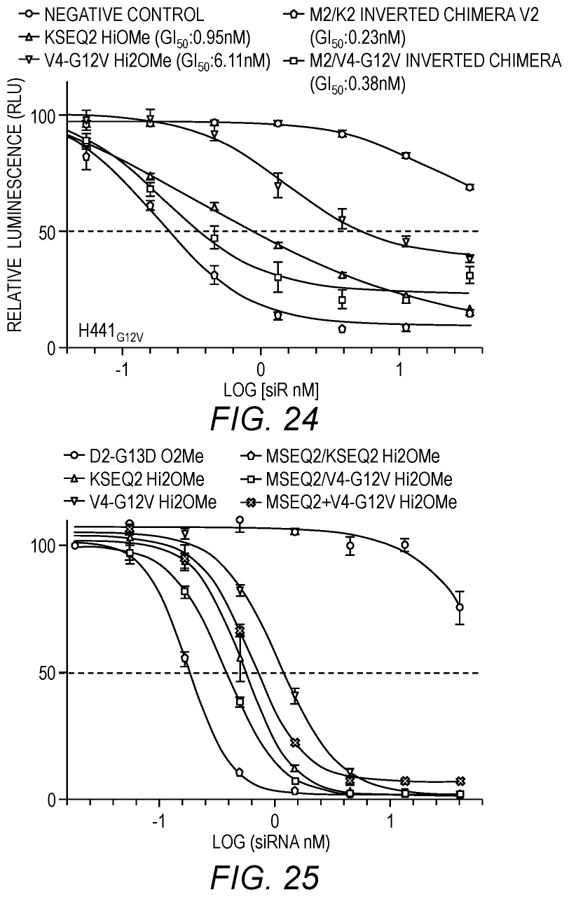

An inverted chimera (M2/V4-G12V) targeting a specific KRAS mutation (G12V) and c-Myc (SEQ ID NOS:75-77)) synergistically decreased cell viability relative to single siRNAs in H441 cells (FIG. 24) and SKCO1 cells (FIG. 25). These data demonstrate that a mutation-specific siRNA (V4-G12V against KRAS G12V mutations) is also unexpectedly potent in the inverted chimeric design, and is more potent against each target than the individual siRNA components.

An inverted chimera targeting IRF4 and c-Myc (SEQ ID NOS:90-92))

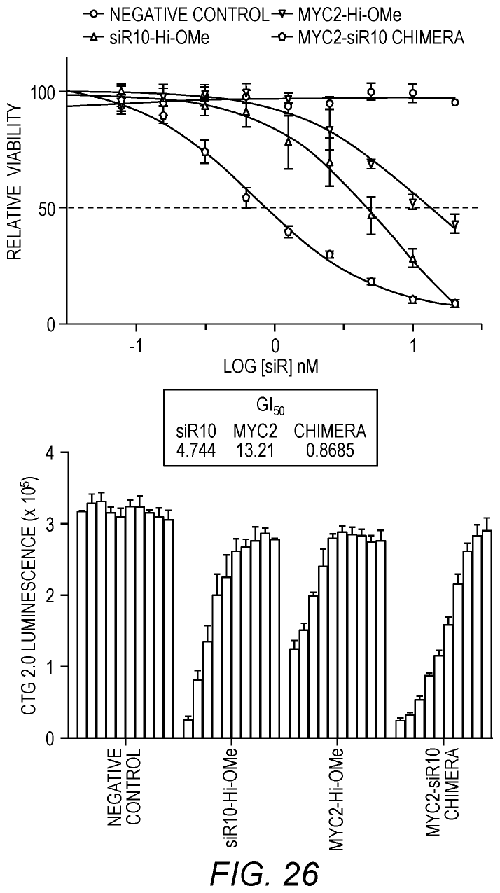

synergistically decreased cell viability relative to single siRNAs in RPMI-8226 myeloma cell line based on a >5-fold improvement in potency than either individual siRNA. These data indicate that the superior effectiveness of inverted chimeras is not target gene or disease type dependent (FIG. 26), and that these designs can be applied to any 2 targets of interest and across different pathophysiologic conditions.

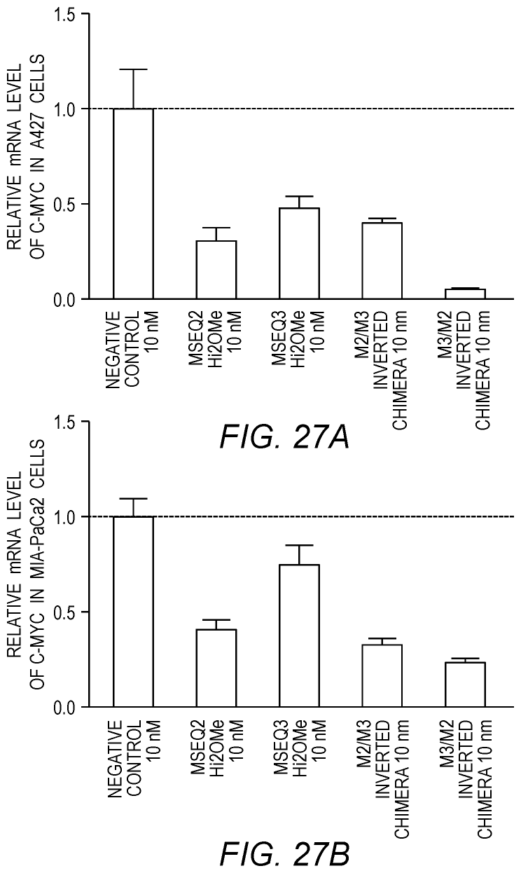

Notably, inverted chimeras comprising two siRNAs targeting the same gene (c-Myc) (SEQ ID NOS:84-86, SEQ ID NOS:87-89)) were more effective at mRNA reduction than targeting the gene with individual siRNAs against the same c-Myc target (FIGS. 27A-27B).

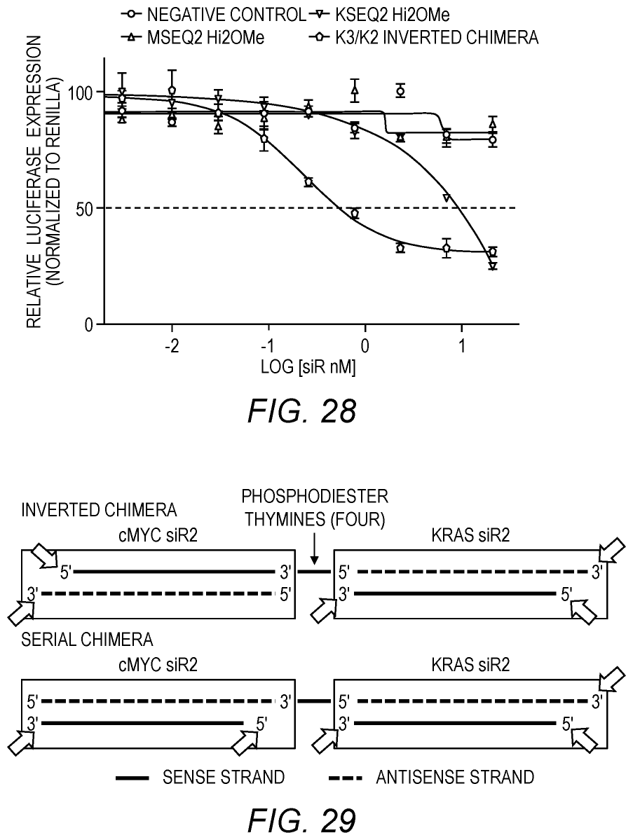

Simialrly, an inverted chimera targeting the same gene (KRAS) was more effective at reducing KRAS-luciferase protein than targeting the gene with individual siRNAs (FIG. 28). Together, these data demonstrate on an mRNA transcript and protein level that inverted chimeras can also be used to more potently silence the same target gene by using siRNAs that silence at unique locations.

All publications, patents, and patent applications are herein incorporated by reference to the same extent as if each individual publication, patent, or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

1. An inverted chimera siRNA molecule comprising a first and second siRNA in opposite orientation, the inverted chimera siRNA molecule comprising:
a) a first RNA molecule comprising a first strand of the first siRNA and a first strand of the second siRNA connected by a linker;
b) a second RNA molecule that is substantially complementary to the first strand of the first siRNA; and
c) a third RNA molecule that is substantially complementary to the first strand of the second siRNA.

2. The inverted chimera siRNA molecule of claim 1, wherein the first RNA molecule comprises the sense strand of the first siRNA and the antisense strand of the second siRNA.

3. The inverted chimera siRNA molecule of claim 1, which is about 30 to about 60 nucleotides in length, such as about 35 to about 55 nucleotides in length, such as about 40 to about 50 nucleotides in length.

4. The inverted chimera siRNA molecule of claim 1, wherein each siRNA independently has a length of about 15 to about 30 nucleotides, such as about 17 to about 25 nucleotides, such as about 19 to about 23 nucleotides.

5. The inverted chimera siRNA molecule of claim 1, wherein the linker is a metabolically-vulnerable linker.

6. The inverted chimera siRNA molecule of claim 1, wherein the linker is about 2 to about 10 nucleotides in length, such as about 3 to about 8 nucleotides in length, such as about 4 to about 6 nucleotides in length.

7. The inverted chimera siRNA molecule of claim 1, wherein the linker comprises phosphodiester thymines, phosphodiester adenines, or a phosphodiester TCA.

8. The inverted chimera siRNA molecule of claim 1 any one of claims 1-7, wherein the molecule comprises at least one chemical modification.

9-11. (canceled)

12. The inverted chimera siRNA molecule of claim 1, wherein the first and second siRNAs bind to the same target gene.

13. The inverted chimera siRNA molecule of claim 1, wherein the first and second siRNAs bind to different target genes.

14. The inverted chimera siRNA molecule of claim 13, wherein one of the first and second siRNAs binds to c-Myc.

15. The inverted chimera siRNA molecule of claim 13, wherein one of the first and second siRNAs binds to c-Myc and the other binds to KRAS.

16. The inverted chimera siRNA molecule of claim 1, further comprising one or more additional siRNAs, each separated by a linker.

17. (canceled)

18. The inverted chimera siRNA molecule of claim 1, wherein the molecule comprises one of the following sets of sequences:
SEQ ID NOS:63-65
SEQ ID NOS:66-68
SEQ ID NOS:69-71
SEQ ID NOS:71-74
SEQ ID NOS:75-77
SEQ ID NOS:78-80
SEQ ID NOS:81-83
SEQ ID NOS:84-86
SEQ ID NOS:87-89
SEQ ID NOS:90-92
SEQ ID NOS:93-95
SEQ ID NOS:96-98
SEQ ID NOS:99-101
SEQ ID NOS: 102-104
or a sequence at least 90% identical thereto.

19-22. (canceled)

23. A pharmaceutical composition comprising the inverted chimera siRNA molecule of claim 1 and a pharmaceutically acceptable carrier.

24. (canceled)

25. A method of inhibiting expression of a target gene in a cell, the method comprising contacting the cell with the inverted chimera siRNA molecule of claim 1, thereby inhibiting expression of the target gene in the cell.

26. A method of treating a disorder associated with a target gene in a subject in need thereof, the method comprising delivering to the subject the inverted chimera siRNA molecule of claim 1, thereby treating the disorder in the subject.

27. A method of treating cancer in a subject in need thereof, wherein the cancer over-expresses a human c-Myc gene, the method comprising delivering to the subject the inverted chimera siRNA molecule of claim 14, thereby treating cancer in the subject.

28. (canceled)

29. A siRNA molecule targeted to a human c-Myc mRNA, wherein the siRNA molecule comprises at least one chemical modification, and wherein the siRNA molecule comprises one of the following pairs of sequences:
sense strand of SEQ ID NO:1 and an antisense strand of SEQ ID NO:19;
sense strand of SEQ ID NO:2 and an antisense strand of SEQ ID NO:20;
sense strand of SEQ ID NO:4 and an antisense strand of SEQ ID NO:22;
sense strand of SEQ ID NO:6 and an antisense strand of SEQ ID NO:24;
sense strand of SEQ ID NO:9 and an antisense strand of SEQ ID NO:27;
or a sequence at least 90% identical thereto.

30-42. (canceled)

43. A siRNA molecule comprising a KRAS siRNA molecule targeted to naturally-occurring human KRAS encoding wild-type KRAS or a mutation selected from G12C, G12D, G12V, or G13D and a c-Myc siRNA molecule targeted to a human c-Myc mRNA, wherein the siRNA molecules comprise at least one chemical modification, and wherein the c-Myc siRNA molecule comprises one of the following pairs of sequences:
sense strand of SEQ ID NO:1 and an antisense strand of SEQ ID NO:19;
sense strand of SEQ ID NO:2 and an antisense strand of SEQ ID NO:20;
sense strand of SEQ ID NO:4 and an antisense strand of SEQ ID NO:22;
sense strand of SEQ ID NO:6 and an antisense strand of SEQ ID NO:24;
sense strand of SEQ ID NO:9 and an antisense strand of SEQ ID NO:27;
or a sequence at least 90% identical thereto.

44-57. (canceled)

58. A siRNA multivalent chimeric molecule comprising:
a KRAS siRNA targeted to naturally-occurring human KRAS encoding wild-type KRAS or a mutation selected from G12C, G12D, G12V, or G13D;
a c-Myc siRNA molecule targeted to human c-Myc mRNA; and
a phosphodiester linker region connecting the KRAS siRNA and the c-Myc siRNA.

59-70. (canceled)


SEQUENCE LISTING (106 sequences)

SEQ ID NO: 1 - RNA, 19 nt, Artificial Sequence
  Synthetic oligonucleotide
gaggauaucuggaagaaau

SEQ ID NO: 2 - RNA, 19 nt, Artificial Sequence
  Synthetic oligonucleotide
gagaacaguugaaacacaa

SEQ ID NO: 3 - RNA, 19 nt, Artificial Sequence
  Synthetic oligonucleotide
aacacaaacuugaacagcu

SEQ ID NO: 4 - RNA, 19 nt, Artificial Sequence
  Synthetic oligonucleotide
aagaagaugaggaagaaau

SEQ ID NO: 5 - RNA, 22 nt, Artificial Sequence
  Synthetic oligonucleotide
cacagcccacugguccucaaga

SEQ ID NO: 6 - RNA, 21 nt, Artificial Sequence
  Synthetic oligonucleotide
aagaggcgaacacacaacguc

SEQ ID NO: 7 - RNA, 17 nt, Artificial Sequence
  Synthetic oligonucleotide
cagaucagcaacaaccg

SEQ ID NO: 8 - RNA, 25 nt, Artificial Sequence
  Synthetic oligonucleotide
gagaccuucaucaaaaacaucauca

SEQ ID NO: 9 - RNA, 19 nt, Artificial Sequence
  Synthetic oligonucleotide
gagcuaaaacggagcuuuu

SEQ ID NO: 10 - DNA, 21 bp, Artificial Sequence
  Synthetic oligonucleotide
gaggauaucuggaagaaautt

SEQ ID NO: 11 - DNA, 21 bp, Artificial Sequence
  Synthetic oligonucleotide
gagaacaguugaaacacaatt

SEQ ID NO: 12 - DNA, 21 bp, Artificial Sequence
  Synthetic oligonucleotide
aacacaaacuugaacagcutt

SEQ ID NO: 13 - DNA, 21 bp, Artificial Sequence
  Synthetic oligonucleotide
aagaagaugaggaagaaautt

SEQ ID NO: 14 - DNA, 24 bp, Artificial Sequence
  Synthetic oligonucleotide
cacagcccacugguccucaagatt

SEQ ID NO: 15 - DNA, 23 bp, Artificial Sequence
  Synthetic oligonucleotide
aagaggcgaacacacaacguctt

SEQ ID NO: 16 - DNA, 19 bp, Artificial Sequence
  Synthetic oligonucleotide
cagaucagcaacaaccgtt

SEQ ID NO: 17 - DNA, 27 bp, Artificial Sequence
  Synthetic oligonucleotide
gagaccuucaucaaaaacaucaucatt

SEQ ID NO: 18 - DNA, 21 bp, Artificial Sequence
  Synthetic oligonucleotide
gagcuaaaacggagcuuuutt

SEQ ID NO: 19 - RNA, 19 nt, Artificial Sequence
  Synthetic oligonucleotide
auuucuuccagauauccuc

SEQ ID NO: 20 - RNA, 19 nt, Artificial Sequence
  Synthetic oligonucleotide
uuguguuucaacuguucuc

SEQ ID NO: 21 - RNA, 19 nt, Artificial Sequence
  Synthetic oligonucleotide
agcuguucaaguuuguguu

SEQ ID NO: 22 - RNA, 19 nt, Artificial Sequence
  Synthetic oligonucleotide
auuucuuccucaucuucuu

SEQ ID NO: 23 - RNA, 22 nt, Artificial Sequence
  Synthetic oligonucleotide
ucuugaggaccagugggcugug

SEQ ID NO: 24 - RNA, 21 nt, Artificial Sequence
  Synthetic oligonucleotide
gacguuguguguucgccucuu

SEQ ID NO: 25 - RNA, 17 nt, Artificial Sequence
  Synthetic oligonucleotide
cgguuguugcugaucug

SEQ ID NO: 26 - RNA, 25 nt, Artificial Sequence
  Synthetic oligonucleotide
ugaugauguuuuugaugaaggucuc

SEQ ID NO: 27 - RNA, 19 nt, Artificial Sequence
  Synthetic oligonucleotide
aaaagcuccguuuuagcuc

SEQ ID NO: 28 - DNA, 21 bp, Artificial Sequence
  Synthetic oligonucleotide
auuucuuccagauauccuctt

SEQ ID NO: 29 - DNA, 21 bp, Artificial Sequence
  Synthetic oligonucleotide
uuguguuucaacuguucuctt

SEQ ID NO: 30 - DNA, 21 bp, Artificial Sequence
  Synthetic oligonucleotide
agcuguucaaguuuguguutt

SEQ ID NO: 31 - DNA, 21 bp, Artificial Sequence
  Synthetic oligonucleotide
auuucuuccucaucuucuutt

SEQ ID NO: 32 - DNA, 24 bp, Artificial Sequence
  Synthetic oligonucleotide
ucuugaggaccagugggcugugtt

SEQ ID NO: 33 - DNA, 23 bp, Artificial Sequence
  Synthetic oligonucleotide
gacguuguguguucgccucuutt

SEQ ID NO: 34 - DNA, 19 bp, Artificial Sequence
  Synthetic oligonucleotide
cgguuguugcugaucugtt

SEQ ID NO: 35 - DNA, 27 bp, Artificial Sequence
  Synthetic oligonucleotide
ugaugauguuuuugaugaaggucuctt

SEQ ID NO: 36 - DNA, 21 bp, Artificial Sequence
  Synthetic oligonucleotide
aaaagcuccguuuuagcuctt

SEQ ID NO: 37 - RNA, 21 nt, Artificial Sequence
  Synthetic oligonucleotide
  modified_base (1)..(1): 2-Fluoroguanosine, phosphorothioate
  modified_base (2)..(2): 2′-O-methylcytidine, phosphorothioate
  modified_base (3)..(3): 2-Fluoroguanosine
  modified_base (4)..(4): 2′-O-methyladenosine
  modified_base (5)..(5): 2′-Fluoroguanosine
  modified_base (6)..(6): 2′-O-methylguanosine
  modified_base (7)..(7): 2′-Fluoroadenosine
  modified_base (8)..(8): 2′-O-methyluridine
  modified_base (9)..(9): 2′-Fluoroadenosine
  modified_base (10)..(10): 2′-Fluorouridine
  modified_base (11)..(11): 2′-Fluorocytidine
  modified_base (12)..(12): 2′-O-methyluridine
  modified_base (13)..(13): 2′-Fluoroguanosine
  modified_base (14)..(14): 2′-O-methylguanosine
  modified_base (15)..(15): 2′-Fluoroadenosine
  modified_base (16)..(16): 2′-O-methyladenosine
  modified_base (17)..(17): 2′-Fluoroguanosine
  modified_base (18)..(18): 2′-O-methyladenosine
  modified_base (19)..(19): 2′-Fluoroadenosine
  modified_base (20)..(20): 2′-O-methyladenosine
  modified_base (21)..(21): 2′-Fluorouridine
gcgaggauaucuggaagaaau

SEQ ID NO: 38 - RNA, 23 nt, Artificial sequence
  Synthetic oligonucleotide
  modified_base (1)..(1): 2′-O-methyladenosine, phosphorothioate
  modified_base (2)..(2): 2′-Fluorouridine, phosphorothioate
  modified_base (3)..(3): 2′-O-methyluridine
  modified_base (4)..(4): 2′-Fluorouridine
  modified_base (5)..(5): 2′-O-methylcytidine
  modified_base (6)..(6): 2′-Fluorouridine
  modified_base (7)..(7): 2′-O-methyluridine
  modified_base (8)..(8): 2′-Fluorocytidine
  modified_base (9)..(9): 2′-O-methylcytidine
  modified_base (10)..(10): 2′-Fluoroadenosine
  modified_base (11)..(11): 2′-O-methylguanosine
  modified_base (12)..(12): 2′-O-methyladenosine
  modified_base (13)..(13): 2′-O-methyluridine
  modified_base (14)..(14): 2′-Fluoroadenosine
  modified_base (15)..(15): 2′-O-methyluridine
  modified_base (16)..(16): 2′-Fluorocytidine
  modified_base (17)..(17): 2′-O-methylcytidine
  modified_base (18)..(18): 2′-Fluorouridine
  modified_base (19)..(19): 2′-O-methylcytidine
  modified_base (20)..(20): 2′-Fluoroguanosine
  modified_base (21)..(21): 2′-O-methylcytidine, phosphorothioate
  modified_base (22)..(22): 2′-O-methyluridine, phosphorothioate
  modified_base (23)..(23): 2′-O-methylguanosine
auuucuuccagauauccucgcug

SEQ ID NO: 39 - RNA, 21 nt, Artificial sequence
  Synthetic oligonucleotide
  modified_base (1)..(1): 2′-Fluoroadenosine, phosphorothioate
  modified_base (2)..(2): 2′-O-methylcytidine, phosphorothioate
  modified_base (3)..(3): 2′-Fluoroguanosine
  modified_base (4)..(4): 2′-O-methyladenosine
  modified_base (5)..(5): 2′-Fluoroguanosine
  modified_base (6)..(6): 2′-O-methyladenosine
  modified_base (7)..(7): 2′-Fluoroadenosine
  modified_base (8)..(8): 2′-O-methylcytidine
  modified_base (9)..(9): 2′-Fluoroadenosine
  modified_base (10)..(10): 2′-Fluoroguanosine
  modified_base (11)..(11): 2′-Fluorouridine
  modified_base (12)..(12): 2′-O-methyluridine
  modified_base (13)..(13): 2′-Fluoroguanosine
  modified_base (14)..(14): 2′-O-methyladenosine
  modified_base (15)..(15): 2′-Fluoroadenosine
  modified_base (16)..(16): 2′-O-methyladenosine
  modified_base (17)..(17): 2′-Fluorocytidine
  modified_base (18)..(18): 2′-O-methyladenosine
  modified_base (19)..(19): 2′-Fluorocytidine
  modified_base (20)..(20): 2′-O-methyladenosine
  modified_base (21)..(21): 2′-Fluoroadenosine
acgagaacaguugaaacacaa

SEQ ID NO: 40 - RNA, 23 nt, Artificial sequence
  Synthetic oligonucleotide
  modified_base (1)..(1): 2′-O-methyluridine, phosphorothioate
  modified_base (2)..(2): 2′-Fluorouridine, phosphorothioate
  modified_base (3)..(3): 2′-O-methylguanosine
  modified_base (4)..(4): 2′-Fluorouridine
  modified_base (5)..(5): 2′-O-methylguanosine
  modified_base (6)..(6): 2′-Fluorouridine
  modified_base (7)..(7): 2′-O-methyluridine
  modified_base (8)..(8): 2′-Fluorouridine
  modified_base (9)..(9): 2′-O-methylcytidine
  modified_base (10)..(10): 2′-Fluoroadenosine
  modified_base (11)..(11): 2′-O-methyladenosine
  modified_base (12)..(12): 2′-O-methylcytidine
  modified_base (13)..(13): 2′-O-methyluridine
  modified_base (14)..(14): 2′-Fluoroguanosine
  modified_base (15)..(15): 2′-O-methyluridine
  modified_base (16)..(16): 2′-Fluorouridine
  modified_base (17)..(17): 2′-O-methylcytidine
  modified_base (18)..(18): 2′-Fluorouridine
  modified_base (19)..(19): 2′-O-methylcytidine
  modified_base (20)..(20): 2′-Fluoroguanosine
  modified_base (21)..(21): 2′-O-methyluridine, phosphorothioate
  modified_base (22)..(22): 2′-O-methylcytidine, phosphorothioate
  modified_base (23)..(23): 2′-O-methylguanosine
uuguguuucaacuguucucgucg

SEQ ID NO: 41 - RNA, 21 nt, Artificial sequence
  Synthetic oligonucleotide
  modified_base (1)..(1): 2′-Fluoroadenosine, phosphorothioate
  modified_base (2)..(2): 2′-O-methylcytidine, phosphorothioate
  modified_base (3)..(3): 2′-Fluoroadenosine
  modified_base (4)..(4): 2′-O-methyladenosine
  modified_base (5)..(5): 2′-Fluoroguanosine
  modified_base (6)..(6): 2′-O-methyladenosine
  modified_base (7)..(7): 2′-Fluoroadenosine
  modified_base (8)..(8): 2′-O-methylguanosine
  modified_base (9)..(9): 2′-Fluoroadenosine
  modified_base (10)..(10): 2′-Fluorouridine
  modified_base (11)..(11): 2′-Fluoroguanosine
  modified_base (12)..(12): 2′-O-methyladenosine
  modified_base (13)..(13): 2′-Fluoroguanosine
  modified_base (14)..(14): 2′-O-methylguanosine
  modified_base (15)..(15): 2′-Fluoroadenosine
  modified_base (16)..(16): 2′-O-methyladenosine
  modified_base (17)..(17): 2′-Fluoroguanosine
  modified_base (18)..(18): 2′-O-methyladenosine
  modified_base (19)..(19): 2′-Fluoroadenosine
  modified_base (20)..(20): 2′-O-methyladenosine
  modified_base (21)..(21): 2′-Fluorouridine
acaagaagaugaggaagaaau

SEQ ID NO: 42 - RNA, 23 nt, Artificial Sequence
  Synthetic oligonucleotide
  modified_base (1)..(1): 2′-O-methyladenosine, phosphorothioate
  modified_base (2)..(2): 2′-Fluorouridine, phosphorothioate
  modified_base (3)..(3): 2′-O-methyluridine
  modified_base (4)..(4): 2′-Fluorouridine
  modified_base (5)..(5): 2′-O-methylcytidine
  modified_base (6)..(6): 2′-Fluorouridine
  modified_base (7)..(7): 2′-O-methyluridine
  modified_base (8)..(8): 2′-Fluorocytidine
  modified_base (9)..(9): 2′-O-methylcytidine
  modified_base (10)..(10): 2′-Fluorouridine
  modified_base (11)..(11): 2′-O-methylcytidine
  modified_base (12)..(12): 2′-O-methyladenosine
  modified_base (13)..(13): 2′-O-methyluridine
  modified_base (14)..(14): 2′-Fluorocytidine
  modified_base (15)..(15): 2′-O-methyluridine
  modified_base (16)..(16): 2′-Fluorouridine
  modified_base (17)..(17): 2′-O-methylcytidine
  modified_base (18)..(18): 2′-Fluorouridine
  modified_base (19)..(19): 2′-O-methyluridine
  modified_base (20)..(20): 2′-Fluoroguanosine
  modified_base (21)..(22): 2′-O-methyluridine, phosphorothioate
  modified_base (23)..(23): 2′-O-methylcytidine
auuucuuccucaucuucuuguuc

SEQ ID NO: 43 - RNA, 21 nt, Artificial sequence
  Synthetic oligonucleotide
  modified_base (1)..(1): 2′-Fluoroadenosine, phosphorothioate
  modified_base (2)..(2): 2′-O-methyladenosine, phosphorothioate
  modified_base (3)..(3): 2′-Fluoroguanosine
  modified_base (4)..(4): 2′-O-methyladenosine
  modified_base (5)..(5): 2′-Fluoroguanosine
  modified_base (6)..(6): 2′-O-methylguanosine
  modified_base (7)..(7): 2′-Fluorocytidine
  modified_base (8)..(8): 2′-O-methylguanosine
  modified_base (9)..(10): 2′-Fluoroadenosine
  modified_base (11)..(11): 2′-Fluorocytidine
  modified_base (12)..(12): 2′-O-methyladenosine
  modified_base (13)..(13): 2′-Fluorocytidine
  modified_base (14)..(14): 2′-O-methyladenosine
  modified_base (15)..(15): 2′-Fluorocytidine
  modified_base (16)..(16): 2′-O-methyladenosine
  modified_base (17)..(17): 2′-Fluoroadenosine
  modified_base (18)..(18): 2′-O-methylcytidine
  modified_base (19)..(19): 2′-Fluoroguanosine
  modified_base (20)..(20): 2′-O-methyluridine
  modified_base (21)..(21): 2′-Fluorocytidine
aagaggcgaacacacaacguc

SEQ ID NO: 44 - RNA, 23 nt, Artificial Sequence
  Synthetic oligonucleotide
  modified_base (1)..(1): 2′-O-methylguanosine, phosphorothioate
  modified_base (2)..(2): 2′-Fluoroadenosine, phosphorothioate
  modified_base (3)..(3): 2′-O-methylcytidine
  modified_base (4)..(4): 2′-Fluoroguanosine
  modified_base (5)..(5): 2′-O-methyluridine
  modified_base (6)..(6): 2′-Fluorouridine
  modified_base (7)..(7): 2′-O-methylguanosine
  modified_base (8)..(8): 2′-Fluorouridine
  modified_base (9)..(9): 2′-O-methylguanosine
  modified_base (10)..(10): 2′-Fluorouridine
  modified_base (11)..(11): 2′-O-methylguanosine
  modified_base (12)..(13): 2′-O-methyluridine
  modified_base (14)..(14): 2′-Fluorocytidine
  modified_base (15)..(15): 2′-O-methylguanosine
  modified_base (16)..(16): 2′-Fluorocytidine
  modified_base (17)..(17): 2′-O-methylcytidine
  modified_base (18)..(18): 2′-Fluorouridine
  modified_base (19)..(19): 2′-O-methylcytidine
  modified_base (20)..(20): 2′-Fluorouridine
  modified_base (21)..(21): 2′-O-methyluridine, phosphorothioate
  modified_base (22)..(22): 2′-O-methylguanosine, phosphorothioate
  modified_base (23)..(23): 2′-O-methyladenosine
gacguuguguguucgccucuuga

SEQ ID NO: 45 - RNA, 21 nt, Artificial Sequence
  Synthetic oligonucleotide
  modified_base (1)..(1): 2′-Fluoroguanosine, phosphorothioate
  modified_base (2)..(2): 2′-O-methyladenosine, phosphorothioate
  modified_base (3)..(3): 2′-Fluoroguanosine
  modified_base (4)..(4): 2′-O-methyladenosine
  modified_base (5)..(5): 2′-Fluoroguanosine
  modified_base (6)..(6): 2′-O-methylcytidine
  modified_base (7)..(7): 2′-Fluorouridine
  modified_base (8)..(8): 2′-O-methyladenosine
  modified_base (9)..(11): 2′-Fluoroadenosine
  modified_base (12)..(12): 2′-O-methylcytidine
  modified_base (13)..(13): 2′-Fluoroguanosine
  modified_base (14)..(14): 2′-O-methylguanosine
  modified_base (15)..(15): 2′-Fluoroadenosine
  modified_base (16)..(16): 2′-O-methylguanosine
  modified_base (17)..(17): 2′-Fluorocytidine
  modified_base (18)..(18): 2′-O-methyluridine
  modified_base (19)..(19): 2′-Fluorouridine
  modified_base (20)..(20): 2′-O-methyluridine
  modified_base (21)..(21): 2′-Fluorouridine
gagagcuaaaacggagcuuuu

SEQ ID NO: 46 - RNA, 23 nt, Artificial Sequence
  Synthetic oligonucleotide
  modified_base (1)..(1): 2′-O-methyladenosine, phosphorothioate
  modified_base (2)..(2): 2′-Fluoroadenosine, phosphorothioate
  modified_base (3)..(3): 2′-O-methyladenosine
  modified_base (4)..(4): 2′-Fluoroadenosine
  modified_base (5)..(5): 2′-O-methylguanosine
  modified_base (6)..(6): 2′-Fluorocytidine
  modified_base (7)..(7): 2′-O-methyluridine
  modified_base (8)..(8): 2′-Fluorocytidine
  modified_base (9)..(9): 2′-O-methylcytidine
  modified_base (10)..(10): 2′-Fluoroguanosine
  modified_base (11)..(13): 2′-O-methyluridine
  modified_base (14)..(14): 2′-Fluorouridine
  modified_base (15)..(15): 2′-O-methyladenosine
  modified_base (16)..(16): 2′-Fluoroguanosine
  modified_base (17)..(17): 2′-O-methylcytidine
  modified_base (18)..(18): 2′-Fluorouridine
  modified_base (19)..(19): 2′-O-methylcytidine
  modified_base (20)..(20): 2′-Fluorouridine
  modified_base (21)..(21): 2′-O-methylcytidine, phosphorothioate
  modified_base (22)..(22): 2′-O-methylguanosine, phosphorothioate
  modified_base (23)..(23): 2′-O-methyluridine
aaaagcuccguuuuagcucucgu

SEQ ID NO: 47 - RNA, 19 nt, Artificial sequence
  Synthetic oligonucleotide
  modified_base (1)..(1): 2′-O-methylguanosine, phosphorothioate
  modified_base (2)..(2): 2′-O-methyluridine, phosphorothioate
  modified_base (3)..(3): 2′-O-methylcytidine
  modified_base (4)..(4): 2′-O-methyluridine
  modified_base (5)..(5): 2′-O-methylcytidine
  modified_base (6)..(6): 2′-O-methyluridine
  modified_base (7)..(7): 2′-Fluorouridine
  modified_base (8)..(8): 2′-O-methylguanosine
  modified_base (9)..(9): 2′-Fluoroguanosine
  modified_base (10)..(10): 2′-Fluoroadenosine
  modified_base (11)..(11): 2′-Fluorouridine
  modified_base (12)..(12): 2′-O-methyladenosine
  modified_base (13)..(14): 2′-O-methyluridine
  modified_base (15)..(15): 2′-O-methylcytidine
  modified_base (16)..(16): 2′-O-methyluridine
  modified_base (17)..(17): 2′-O-methylcytidine
  modified_base (18)..(18): 2′-O-methylguanosine
  modified_base (19)..(19): 2′-O-methyladenosine
gucucuuggauauucucga

SEQ ID NO: 48 - RNA, 21 nt, Artificial Sequence
  Synthetic oligonucleotide
  modified_base (1)..(1): 2′-O-methyluridine, phosphorothioate
  modified_base (2)..(2): 2′-Fluorocytidine, phosphorothioate
  modified_base (3)..(3): 2′-O-methylguanosine
  modified_base (4)..(4): 2′-O-methyladenosine
  modified_base (5)..(5): 2′-O-methylguanosine
  modified_base (6)..(6): 2′-Fluoroadenosine
  modified_base (7)..(7): 2′-O-methyladenosine
  modified_base (8)..(8): 2′-O-methyluridine
  modified_base (9)..(9): 2′-O-methyladenosine
  modified_base (10)..(10): 2′-O-methyluridine
  modified_base (11)..(12): 2′-O-methylcytidine
  modified_base (13)..(13): 2′-O-methyladenosine
  modified_base (14)..(14): 2′-Fluoroadenosine
  modified_base (15)..(15): 2′-O-methylguanosine
  modified_base (16)..(16): 2′-Fluoroadenosine
  modified_base (17)..(17): 2′-O-methylguanosine
  modified_base (18)..(18): 2′-O-methyladenosine
  modified_base (19)..(19): 2′-O-methylcytidine, phosphorothioate
  modified_base (20)..(20): 2′-O-methyladenosine, phosphorothioate
  modified_base (21)..(21): 2′-O-methylguanosine
ucgagaauauccaagagacag

SEQ ID NO: 49 - RNA, 19 nt, Artificial Sequence
  Synthetic oligonucleotide
  modified_base (1)..(1): 2′-O-methylcytidine, phosphorothioate
  modified_base (2)..(2): 2′-O-methyladenosine, phosphorothioate
  modified_base (3)..(3): 2′-O-methylguanosine
  modified_base (4)..(4): 2′-O-methylcytidine
  modified_base (5)..(5): 2′-O-methyluridine
  modified_base (6)..(6): 2′-O-methyladenosine
  modified_base (7)..(7): 2′-Fluoroadenosine
  modified_base (8)..(8): 2′-O-methyluridine
  modified_base (9)..(9): 2′-Fluorouridine
  modified_base (10)..(10): 2′-Fluorocytidine
  modified_base (11)..(11): 2′-Fluoroadenosine
  modified_base (12)..(12): 2′-O-methylguanosine
  modified_base (13)..(14): 2′-O-methyladenosine
  modified_base (15)..(15): 2′-O-methyluridine
  modified_base (16)..(16): 2′-O-methylcytidine
  modified_base (17)..(17): 2′-O-methyladenosine
  modified_base (18)..(19): 2′-O-methyluridine
cagcuaauucagaaucauu

SEQ ID NO: 50 - RNA, 21 nt, Artificial Sequence
  Synthetic oligonucleotide
  modified_base (1)..(1): 2′-O-methyladenosine, phosphorothioate
  modified_base (2)..(2): 2′-Fluoroadenosine, phosphorothioate
  modified_base (3)..(3): 2′-O-methyluridine
  modified_base (4)..(4): 2′-O-methylguanosine
  modified_base (5)..(5): 2′-O-methyladenosine
  modified_base (6)..(6): 2′-Fluorouridine
  modified_base (7)..(7): 2′-O-methyluridine
  modified_base (8)..(8): 2′-O-methylcytidine
  modified_base (9)..(9): 2′-O-methyluridine
  modified_base (10)..(10): 2′-O-methylguanosine
  modified_base (11)..(12): 2′-O-methyladenosine
  modified_base (13)..(13): 2′-O-methyluridine
  modified_base (14)..(14): 2′-Fluorouridine
  modified_base (15)..(15): 2′-O-methyladenosine
  modified_base (16)..(16): 2′-Fluoroguanosine
  modified_base (17)..(17): 2′-O-methylcytidine
  modified_base (18)..(18): 2′-O-methyluridine
  modified_base (19)..(19): 2′-O-methylguanosine, phosphorothioate
  modified_base (20)..(20): 2′-O-methyluridine, phosphorothioate
  modified_base (21)..(21): 2′-O-methyladenosine
aaugauucugaauuagcugua

SEQ ID NO: 51 - RNA, 21 nt, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(3): 2′-O-methylguanosine
  modified_base (4)..(4): 2′-O-methyladenosine
  modified_base (5)..(5): 2′-O-methylguanosine
  modified_base (6)..(6): 2′-O-methyladenosine
  modified_base (7)..(7): 2′-Fluoroadenosine
  modified_base (8)..(8): 2′-O-methylcytidine
  modified_base (9)..(9): 2′-Fluoroadenosine
  modified_base (10)..(10): 2′-Fluoroguanosine
  modified_base (11)..(11): 2′-Fluorouridine
  modified_base (12)..(12): 2′-O-methyluridine
  modified_base (13)..(13): 2′-O-methylguanosine
  modified_base (14)..(16): 2′-O-methyladenosine
  modified_base (17)..(17): 2′-O-methylcytidine
  modified_base (18)..(18): 2′-O-methyladenosine
  modified_base (19)..(19): 2′-O-methylcytidine, phosphorothioate
  modified_base (20)..(20): 2′-O-methyladenosine, phosphorothioate
  modified_base (21)..(21): 2′-O-methyladenosine
acgagaacaguugaaacacaa

SEQ ID NO: 52 - RNA, 23 nt, Artificial Sequence
  Synthetic oligonucleotide
  modified_base (1)..(1): 2′-O-methyluridine, phosphorothioate
  modified_base (2)..(2): 2′-Fluorouridine, phosphorothioate
  modified_base (3)..(3): 2′-O-methylguanosine
  modified_base (4)..(4): 2′-O-methyluridine
  modified_base (5)..(5): 2′-O-methylguanosine
  modified_base (6)..(6): 2′-Fluorouridine
  modified_base (7)..(8): 2′-O-methyluridine
  modified_base (9)..(9): 2′-O-methylcytidine
  modified_base (10)..(11): 2′-O-methyladenosine
  modified_base (12)..(12): 2′-O-methylcytidine
  modified_base (13)..(13): 2′-O-methyluridine
  modified_base (14)..(14): 2′-Fluoroguanosine
  modified_base (15)..(15): 2′-O-methyluridine
  modified_base (16)..(16): 2′-Fluorouridine
  modified_base (17)..(17): 2′-O-methylcytidine
  modified_base (18)..(18): 2′-O-methyluridine
  modified_base (19)..(19): 2′-O-methylcytidine
  modified_base (20)..(20): 2′-O-methylguanosine
  modified_base (21)..(21): 2′-O-methyluridine, phosphorothioate
  modified_base (22)..(22): 2′-O-methylcytidine, phosphorothioate
  modified_base (23)..(23): 2′-O-methylguanosine
uuguguuucaacuguucucgucg

SEQ ID NO: 53 - RNA, 21 nt, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(4): 2′-O-methyladenosine
  modified_base (5)..(5): 2′-O-methylguanosine
  modified_base (6)..(6): 2′-O-methyladenosine
  modified_base (7)..(7): 2′-Fluoroadenosine
  modified_base (8)..(8): 2′-O-methylguanosine
  modified_base (9)..(9): 2′-Fluoroadenosine
  modified_base (10)..(10): 2′-Fluorouridine
  modified_base (11)..(11): 2′-Fluoroguanosine
  modified_base (12)..(12): 2′-O-methyladenosine
  modified_base (13)..(14): 2′-O-methylguanosine
  modified_base (15)..(16): 2′-O-methyladenosine
  modified_base (17)..(17): 2′-O-methylguanosine
  modified_base (18)..(18): 2′-O-methyladenosine
  modified_base (19)..(20): 2′-O-methyladenosine, phosphorothioate
  modified_base (21)..(21): 2′-O-methyluridine
acaagaagaugaggaagaaau

SEQ ID NO: 54 - RNA, 23 nt, Artificial Sequence
  Synthetic oligonucleotide
  modified_base (1)..(1): 2′-O-methyladenosine, phosphorothioate
  modified_base (2)..(2): 2′-Fluorouridine, phosphorothioate
  modified_base (3)..(4): 2′-O-methyluridine
  modified_base (5)..(5): 2′-O-methylcytidine
  modified_base (6)..(6): 2′-Fluorouridine
  modified_base (7)..(7): 2′-O-methyluridine
  modified_base (8)..(9): 2′-O-methylcytidine
  modified_base (9)..(9): 2′-O-methyluridine
  modified_base (10)..(10): 2′-O-methyluridine
  modified_base (11)..(11): 2′-O-methylcytidine
  modified_base (12)..(12): 2′-O-methyladenosine
  modified_base (13)..(13): 2′-O-methyluridine
  modified_base (14)..(14): 2′-Fluorocytidine
  modified_base (15)..(15): 2′-O-methyluridine
  modified_base (16)..(16): 2′-Fluorouridine
  modified_base (17)..(17): 2′-O-methylcytidine
  modified_base (18)..(19): 2′-O-methyluridine
  modified_base (20)..(20): 2′-O-methylguanosine
  modified_base (21)..(22): 2′-O-methyluridine, phosphorothioate
  modified_base (23)..(23): 2′-O-methylcytidine
auuucuuccucaucuucuuguuc

SEQ ID NO: 55 - RNA, 21 nt, Artificial Sequence
  Synthetic oligonucleotide
  modified_base (1)..(1): 2′-O-methylguanosine, phosphorothioate
  modified_base (2)..(2): 2′-O-methyluridine, phosphorothioate
  modified_base (3)..(4): 2′-O-methylguanosine
  modified_base (5)..(5): 2′-O-methyluridine
  modified_base (6)..(6): 2′-O-methylguanosine
  modified_base (7)..(7): 2′-Fluoroguanosine
  modified_base (8)..(8): 2′-O-methyluridine
  modified_base (9)..(9): 2′-Fluorouridine
  modified_base (10)..(11): 2′-Fluoroguanosine
  modified_base (12)..(12): 2′-O-methyladenosine
  modified_base (13)..(13): 2′-O-methylguanosine
  modified_base (14)..(14): 2′-O-methylcytidine
  modified_base (15)..(15): 2′-O-methyluridine
  modified_base (16)..(16): 2′-O-methylguanosine
  modified_base (17)..(18): 2′-O-methyluridine
  modified_base (19)..(20): 2′-O-methylguanosine, phosphorothioate
  modified_base (21)..(21): 2′-O-methylcytidine
gugguaguuggagcuguuggc

SEQ ID NO: 56 - RNA, 23 nt, Artificial Sequence
  Synthetic oligonucleotide
  modified_base (1)..(1): 2′-O-methylguanosine, phosphorothioate
  modified_base (2)..(2): 2′-Fluorocytidine, phosphorothioate
  modified_base (3)..(3): 2′-O-methylcytidine
  modified_base (4)..(5): 2′-O-methyladenosine
  modified_base (6)..(6): 2′-Fluorocytidine
  modified_base (7)..(7): 2′-O-methyladenosine
  modified_base (8)..(8): 2′-O-methylguanosine
  modified_base (9)..(9): 2′-O-methylcytidine
  modified_base (10)..(10): 2′-O-methyluridine
  modified_base (11)..(12): 2′-O-methylcytidine
  modified_base (13)..(13): 2′-O-methyladenosine
  modified_base (14)..(14): 2′-Fluoroadenosine
  modified_base (15)..(15): 2′-O-methylcytidine
  modified_base (16)..(16): 2′-Fluorouridine
  modified_base (17)..(17): 2′-O-methyladenosine
  modified_base (18)..(19): 2′-O-methylcytidine
  modified_base (20)..(20): 2′-O-methyladenosine
  modified_base (21)..(21): 2′-O-methylcytidine, phosphorothioate
  modified_base (22)..(22): 2′-O-methyladenosine, phosphorothioate
  modified_base (23)..(23): 2′-O-methyladenosine
gccaacagcuccaacuaccacaa

SEQ ID NO: 57 - RNA, 21 nt, Artificial Sequence
  Synthetic oligonucleotide
  modified_base (1)..(1): 2′-O-methyluridine, phosphorothioate
  modified_base (2)..(2): 2′-O-methyladenosine, phosphorothioate
  modified_base (3)..(3): 2′-O-methyladenosine
  modified_base (4)..(4): 2′-O-methylcytidine
  modified_base (5)..(6): 2′-O-methyladenosine
  modified_base (7)..(7): 2′-Fluorouridine
  modified_base (8)..(8): 2′-O-methylguanosine
  modified_base (9)..(10): 2′-Fluorouridine
  modified_base (11)..(11): 2′-Fluoroguanosine
  modified_base (12)..(12): 2′-O-methylcytidine
  modified_base (13)..(16): 2′-O-methyluridine
  modified_base (17)..(17): 2′-O-methylcytidine
  modified_base (18)..(18): 2′-O-methyladenosine
  modified_base (19)..(20): 2′-O-methyladenosine, phosphorothioate
  modified_base (21)..(21): 2′-O-methyladenosine
uaacaauguugcuuuucaaaa

SEQ ID NO: 58 - RNA, 23 nt, Artificial Sequence
  Synthetic oligonucleotide
  modified_base (1)..(1): 2′-O-methyluridine, phosphorothioate
  modified_base (2)..(2): 2′-Fluorouridine, phosphorothioate
  modified_base (3)..(4): 2′-O-methyluridine
  modified_base (5)..(5): 2′-O-methylguanosine
  modified_base (6)..(6): 2′-Fluoroadenosine
  modified_base (7)..(9): 2′-O-methyladenosine
  modified_base (10)..(10): 2′-O-methylguanosine
  modified_base (11)..(11): 2′-O-methylcytidine
  modified_base (12)..(13): 2′-O-methyladenosine
  modified_base (14)..(14): 2′-Fluorocytidine
  modified_base (15)..(15): 2′-O-methyladenosine
  modified_base (16)..(16): 2′-Fluorouridine
  modified_base (17)..(17): 2′-O-methyluridine
  modified_base (18)..(18): 2′-O-methylguanosine
  modified_base (19)..(20): 2′-O-methyluridine
  modified_base (21)..(21): 2′-O-methyladenosine, phosphorothioate
  modified_base (22)..(22): 2′-O-methylguanosine, phosphorothioate
  modified_base (23)..(23): 2′-O-methyluridine
uuuugaaaagcaacauuguuagu

SEQ ID NO: 59 - RNA, 21 nt, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(3): 2′-O-methyluridine
  modified_base (4)..(4): 2′-O-methylguanosine
  modified_base (5)..(5): 2′-O-methyluridine
  modified_base (6)..(6): 2′-O-methyladenosine
  modified_base (7)..(7): 2′-Fluorocytidine
  modified_base (8)..(8): 2′-O-methyladenosine
  modified_base (9)..(9): 2′-Fluoroguanosine
  modified_base (10)..(10): 2′-Fluorouridine
  modified_base (11)..(11): 2′-Fluorocytidine
  modified_base (12)..(12): 2′-O-methyluridine
  modified_base (13)..(16): 2′-O-methyladenosine
  modified_base (17)..(17): 2′-O-methyluridine
  modified_base (18)..(18): 2′-O-methylguanosine
  modified_base (19)..(19): 2′-O-methyluridine, phosphorothioate
  modified_base (20)..(20): 2′-O-methylcytidine, phosphorothioate
  modified_base (21)..(21): 2′-O-methyladenosine
acuguacagucuaaaauguca

SEQ ID NO: 60 - RNA, 23 nt, Artificial Sequence
  Synthetic oligonucleotide
  modified_base (1)..(1): 2′-O-methyluridine, phosphorothioate
  modified_base (2)..(2): 2′-Fluoroguanosine, phosphorothioate
  modified_base (3)..(3): 2′-O-methyladenosine
  modified_base (4)..(4): 2′-O-methylcytidine
  modified_base (5)..(5): 2′-O-methyladenosine
  modified_base (6)..(6): 2′-Fluorouridine
  modified_base (7)..(9): 2′-O-methyluridine
  modified_base (10)..(10): 2′-O-methyladenosine
  modified_base (11)..(11): 2′-O-methylguanosine
  modified_base (12)..(12): 2′-O-methyladenosine
  modified_base (13)..(13): 2′-O-methylcytidine
  modified_base (14)..(14): 2′-Fluorouridine
  modified_base (15)..(15): 2′-O-methylguanosine
  modified_base (16)..(16): 2′-Fluorouridine
  modified_base (17)..(17): 2′-O-methyladenosine
  modified_base (18)..(18): 2′-O-methylcytidine
  modified_base (19)..(19): 2′-O-methyladenosine
  modified_base (20)..(20): 2′-O-methylguanosine
  modified_base (21)..(21): 2′-O-methyluridine, phosphorothioate
  modified_base (22)..(22): 2′-O-methylguanosine, phosphorothioate
  modified_base (23)..(23): 2′-O-methylguanosine
ugacauuuuagacuguacagugg

SEQ ID NO: 61 - RNA, 21 nt, Artificial Sequence
  Synthetic oligonucleotide
  modified_base (1)..(1): 2′-O-methylguanosine, phosphorothioate
  modified_base (2)..(2): 2′-O-methyluridine, phosphorothioate
  modified_base (3)..(3): 2′-O-methyladenosine
  modified_base (4)..(4): 2′-O-methylguanosine
  modified_base (5)..(6): 2′-O-methyluridine
  modified_base (7)..(8): 2′-O-methylguanosine
  modified_base (9)..(9): 2′-O-methyladenosine
  modified_base (10)..(10): 2′-O-methylguanosine
  modified_base (11)..(11): 2′-O-methylcytidine
  modified_base (12)..(12): 2′-O-methyluridine
  modified_base (13)..(16): 2′-O-methylguanosine
  modified_base (17)..(17): 2′-O-methyladenosine
  modified_base (18)..(18): 2′-O-methylcytidine
  modified_base (19)..(19): 2′-O-methylguanosine
  modified_base (20)..(20): 2′-O-methyluridine
  modified_base (21)..(21): 2′-O-methyladenosine
guaguuggagcuggggacgua

SEQ ID NO: 62 - RNA, 23 nt, Artificial Sequence
  Synthetic oligonucleotide
  modified_base (1)..(1): 2′-O-methyluridine, phosphorothioate
  modified_base (2)..(2): 2′-O-methyladenosine, phosphorothioate
  modified_base (3)..(3): 2′-O-methylcytidine
  modified_base (4)..(4): 2′-O-methylguanosine
  modified_base (5)..(5): 2′-O-methyluridine
  modified_base (6)..(6): 2′-O-methylcytidine
  modified_base (7)..(7): 2′-O-methyladenosine
  modified_base (8)..(9): 2′-O-methylcytidine
  modified_base (10)..(10): 2′-O-methyladenosine
  modified_base (11)..(11): 2′-O-methylguanosine
  modified_base (12)..(12): 2′-O-methylcytidine
  modified_base (13)..(13): 2′-O-methyluridine
  modified_base (14)..(15): 2′-O-methylcytidine
  modified_base (16)..(17): 2′-O-methyladenosine
  modified_base (18)..(18): 2′-O-methylcytidine
  modified_base (19)..(19): 2′-O-methyluridine
  modified_base (20)..(20): 2′-O-methyladenosine
  modified_base (21)..(22): 2′-O-methylcytidine, phosphorothioate
  modified_base (23)..(23): 2′-O-methyladenosine
uacgucaccagcuccaacuacca

SEQ ID NO: 63 - DNA, 46 bp, Artificial Sequence
  Synthetic oligonucleotide
  modified_base (1)..(1): 2′-Fluoroadenosine, phosphorothioate
  modified_base (2)..(2): 2′-O-methylcytidine, phosphorothioate
  modified_base (3)..(3): 2′-Fluoroguanosine
  modified_base (4)..(4): 2′-O-methyladenosine
  modified_base (5)..(5): 2′-Fluoroguanosine
  modified_base (6)..(6): 2′-O-methyladenosine
  modified_base (7)..(7): 2′-Fluoroadenosine
  modified_base (8)..(8): 2′-O-methylcytidine
  modified_base (9)..(9): 2′-Fluoroadenosine
  modified_base (10)..(10): 2′-Fluoroguanosine
  modified_base (11)..(11): 2′-Fluorouridine
  modified_base (12)..(12): 2′-O-methyluridine
  modified_base (13)..(13): 2′-Fluoroguanosine
  modified_base (14)..(14): 2′-O-methyladenosine
  modified_base (15)..(15): 2′-Fluoroadenosine
  modified_base (16)..(16): 2′-O-methyladenosine
  modified_base (17)..(17): 2′-Fluorocytidine
  modified_base (18)..(18): 2′-O-methyladenosine
  modified_base (19)..(19): 2′-Fluorocytidine, phosphorothioate
  modified_base (20)..(20): 2′-O-methyladenosine, phosphorothioate
  modified_base (21)..(21): 2′-Fluoroadenosine
  modified_base (26)..(26): 2′-O-methyluridine, phosphorothioate
  modified_base (27)..(27): 2′-Fluorocytidine, phosphorothioate
  modified_base (28)..(28): 2′-O-methylguanosine
  modified_base (29)..(29): 2′-O-methyladenosine
  modified_base (30)..(30): 2′-O-methylguanosine
  modified_base (31)..(31): 2′-Fluoroadenosine
  modified_base (32)..(32): 2′-O-methyladenosine
  modified_base (33)..(33): 2′-O-methyluridine
  modified_base (34)..(34): 2′-O-methyladenosine
  modified_base (35)..(35): 2′-O-methyluridine
  modified_base (36)..(37): 2′-O-methylcytidine
  modified_base (38)..(38): 2′-O-methyladenosine
  modified_base (39)..(39): 2′-Fluoroadenosine
  modified_base (40)..(40): 2′-O-methylguanosine
  modified_base (41)..(41): 2′-Fluoroadenosine
  modified_base (42)..(42): 2′-O-methylguanosine
  modified_base (43)..(43): 2′-O-methyladenosine
  modified_base (44)..(44): 2′-O-methylcytidine, phosphorothioate
  modified_base (46)..(46): 2′-O-methylguanosine
acgagaacaguugaaacacaattttucgagaauauccaagagacag

SEQ ID NO: 64 - RNA, 23 nt, Artificial Sequence
  Synthetic oligonucleotide
  modified_base (1)..(1): 2′-O-methyluridine, phosphorothioate
  modified_base (2)..(2): 2′-Fluorouridine, phosphorothioate
  modified_base (3)..(3): 2′-O-methylguanosine
  modified_base (4)..(4): 2′-Fluorouridine
  modified_base (5)..(5): 2′-O-methylguanosine
  modified_base (6)..(6): 2′-Fluorouridine
  modified_base (7)..(7): 2′-O-methyluridine
  modified_base (8)..(8): 2′-Fluorouridine
  modified_base (9)..(9): 2′-O-methylcytidine
  modified_base (10)..(10): 2′-Fluoroadenosine
  modified_base (11)..(11): 2′-O-methyladenosine
  modified_base (12)..(12): 2′-O-methylcytidine
  modified_base (13)..(13): 2′-O-methyluridine
  modified_base (14)..(14): 2′-Fluoroguanosine
  modified_base (15)..(15): 2′-O-methyluridine
  modified_base (16)..(16): 2′-Fluorouridine
  modified_base (17)..(17): 2′-O-methylcytidine
  modified_base (18)..(18): 2′-Fluorouridine
  modified_base (19)..(19): 2′-O-methylcytidine
  modified_base (20)..(20): 2′-Fluoroguanosine
  modified_base (21)..(21): 2′-O-methyluridine, phosphorothioate
  modified_base (22)..(22): 2′-O-methylcytidine, phosphorothioate
  modified_base (23)..(23): 2′-O-methylguanosine
uuguguuucaacuguucucgucg

SEQ ID NO: 65 - RNA, 19 nt, Artificial Sequence
  Synthetic oligonucleotide
  modified_base (1)..(1): 2′-O-methylguanosine, phosphorothioate
  modified_base (2)..(2): 2′-O-methyluridine, phosphorothioate
  modified_base (3)..(3): 2′-O-methylcytidine
  modified_base (4)..(4): 2′-O-methyluridine
  modified_base (5)..(5): 2′-O-methylcytidine
  modified_base (6)..(6): 2′-O-methyluridine
  modified_base (7)..(7): 2′-Fluorouridine
  modified_base (8)..(8): 2′-O-methylguanosine
  modified_base (9)..(9): 2′-Fluoroguanosine
  modified_base (10)..(10): 2′-Fluoroadenosine
  modified_base (11)..(11): 2′-Fluorouridine
  modified_base (12)..(12): 2′-O-methyladenosine
  modified_base (13)..(14): 2′-O-methyluridine
  modified_base (15)..(15): 2′-O-methylcytidine
  modified_base (16)..(16): 2′-O-methyluridine
  modified_base (17)..(17): 2′-O-methylcytidine
  modified_base (18)..(18): 2′-O-methylguanosine
  modified_base (19)..(19): 2′-O-methyladenosine
gucucuuggauauucucga

SEQ ID NO: 66 - DNA, 48 bp, Artificial Sequence
  Synthetic oligonucleotide
  modified_base (1)..(1): 2′-O-methyluridine, phosphorothioate
  modified_base (2)..(2): 2′-Fluorouridine, phosphorothioate
  modified_base (3)..(3): 2′-O-methylguanosine
  modified_base (4)..(4): 2′-Fluorouridine
  modified_base (5)..(5): 2′-O-methylguanosine
  modified_base (6)..(6): 2′-Fluorouridine
  modified_base (7)..(7): 2′-O-methyluridine
  modified_base (8)..(8): 2′-Fluorouridine
  modified_base (9)..(9): 2′-O-methylcytidine
  modified_base (10)..(10): 2′-Fluoroadenosine
  modified_base (11)..(11): 2′-O-methyladenosine
  modified_base (12)..(12): 2′-O-methylcytidine
  modified_base (13)..(13): 2′-O-methyluridine
  modified_base (14)..(14): 2′-Fluoroguanosine
  modified_base (15)..(15): 2′-O-methyluridine
  modified_base (16)..(16): 2′-Fluorouridine
  modified_base (17)..(17): 2′-O-methylcytidine
  modified_base (18)..(18): 2′-Fluorouridine
  modified_base (19)..(19): 2′-O-methylcytidine
  modified_base (20)..(20): 2′-Fluoroguanosine
  modified_base (21)..(21): 2′-O-methyluridine, phosphorothioate
  modified_base (22)..(22): 2′-O-methylcytidine, phosphorothioate
  modified_base (23)..(23): 2′-O-methylguanosine
  modified_base (28)..(28): 2′-O-methyluridine, phosphorothioate
  modified_base (29)..(29): 2′-Fluorocytidine, phosphorothioate
  modified_base (30)..(30): 2′-O-methylguanosine
  modified_base (31)..(31): 2′-O-methyladenosine
  modified_base (32)..(32): 2′-O-methylguanosine
  modified_base (33)..(33): 2′-Fluoroadenosine
  modified_base (34)..(34): 2′-Fluoroadenosine
  modified_base (35)..(35): 2′-O-methyluridine
  modified_base (36)..(36): 2′-O-methyladenosine
  modified_base (37)..(37): 2′-O-methyluridine
  modified_base (38)..(39): 2′-O-methylcytidine
  modified_base (40)..(40): 2′-O-methyladenosine
  modified_base (41)..(41): 2′-Fluoroadenosine
  modified_base (42)..(42): 2′-O-methylguanosine
  modified_base (43)..(43): 2′-Fluoroadenosine
  modified_base (44)..(44): 2′-O-methylguanosine
  modified_base (45)..(45): 2′-O-methyladenosine
  modified_base (46)..(46): 2′-O-methylcytidine, phosphorothioate
  modified_base (47)..(47): 2′-O-methyladenosine, phosphorothioate
  modified_base (48)..(48): 2′-O-methylguanosine
uuguguuucaacuguucucgucgttttucgagaauauccaagagacag

SEQ ID NO: 67 - RNA, 21 nt, Artificial Sequence
  Synthetic oligonucleotide
  modified_base (1)..(1): 2′-Fluoroadenosine, phosphorothioate
  modified_base (2)..(2): 2′-O-methylcytidine, phosphorothioate
  modified_base (3)..(3): 2′-Fluoroguanosine
  modified_base (4)..(4): 2′-O-methyladenosine
  modified_base (5)..(5): 2′-Fluoroguanosine
  modified_base (6)..(6): 2′-O-methyladenosine
  modified_base (7)..(7): 2′-Fluoroadenosine
  modified_base (8)..(8): 2′-O-methylcytidine
  modified_base (9)..(9): 2′-Fluoroadenosine
  modified_base (10)..(10): 2′-Fluoroguanosine
  modified_base (11)..(11): 2′-Fluorouridine
  modified_base (12)..(12): 2′-O-methyluridine
  modified_base (13)..(13): 2′-Fluoroguanosine
  modified_base (14)..(14): 2′-O-methyladenosine
  modified_base (15)..(15): 2′-Fluoroadenosine
  modified_base (16)..(16): 2′-O-methyladenosine
  modified_base (17)..(17): 2′-Fluorocytidine
  modified_base (18)..(18): 2′-O-methyladenosine
  modified_base (19)..(19): 2′-Fluorocytidine, phosphorothioate
  modified_base (20)..(20): 2′-O-methyladenosine, phosphorothioate
  modified_base (21)..(21): 2′-Fluoroadenosine
acgagaacaguugaaacacaa

SEQ ID NO: 68 - RNA, 19 nt, Artificial Sequence
  Synthetic oligonucleotide
  modified_base (1)..(1): 2′-O-methylguanosine, phosphorothioate
  modified_base (2)..(2): 2′-O-methyluridine, phosphorothioate
  modified_base (3)..(3): 2′-O-methylcytidine
  modified_base (4)..(4): 2′-O-methyluridine
  modified_base (5)..(5): 2′-O-methylcytidine
  modified_base (6)..(6): 2′-O-methyluridine
  modified_base (7)..(7): 2′-Fluorouridine
  modified_base (8)..(8): 2′-O-methylguanosine
  modified_base (9)..(9): 2′-Fluoroguanosine
  modified_base (10)..(10): 2′-Fluoroadenosine
  modified_base (11)..(11): 2′-Fluorouridine
  modified_base (12)..(12): 2′-O-methyladenosine
  modified_base (13)..(14): 2′-O-methyluridine
  modified_base (15)..(15): 2′-O-methylcytidine
  modified_base (16)..(16): 2′-O-methyluridine
  modified_base (17)..(17): 2′-O-methylcytidine, phosphorothioate
  modified_base (18)..(18): 2′-O-methylguanosine, phosphorothioate
  modified_base (19)..(19): 2′-O-methyladenosine
gucucuuggauauucucga

SEQ ID NO: 69 - DNA, 46 bp, Artificial Sequence; identical to SEQ ID NO: 63
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(3): 2′-O-methylguanosine
  modified_base (4)..(4): 2′-O-methyladenosine
  modified_base (5)..(5): 2′-O-methylguanosine
  modified_base (6)..(6): 2′-O-methyladenosine
  modified_base (7)..(7): 2′-Fluoroadenosine
  modified_base (8)..(8): 2′-O-methylcytidine
  modified_base (9)..(9): 2′-Fluoroadenosine
  modified_base (10)..(10): 2′-Fluoroguanosine
  modified_base (11)..(11): 2′-Fluorouridine
  modified_base (12)..(12): 2′-O-methyluridine
  modified_base (13)..(13): 2′-O-methylguanosine
  modified_base (14)..(16): 2′-O-methyladenosine
  modified_base (17)..(17): 2′-O-methylcytidine
  modified_base (18)..(18): 2′-O-methyladenosine
  modified_base (19)..(19): 2′-O-methylcytidine, phosphorothioate
  modified_base (20)..(20): 2′-O-methyladenosine, phosphorothioate
  modified_base (21)..(21): 2′-O-methyladenosine
  modified_base (26)..(26): 2′-O-methyluridine, phosphorothioate
  modified_base (27)..(27): 2′-Fluorocytidine, phosphorothioate
  modified_base (28)..(28): 2′-O-methylguanosine
  modified_base (29)..(29): 2′-O-methyladenosine
  modified_base (30)..(30): 2′-O-methylguanosine
  modified_base (31)..(31): 2′-Fluoroadenosine
  modified_base (32)..(32): 2′-O-methyladenosine
  modified_base (33)..(33): 2′-O-methyluridine
  modified_base (34)..(34): 2′-O-methyladenosine
  modified_base (35)..(35): 2′-O-methyluridine
  modified_base (36)..(37): 2′-O-methylcytidine
  modified_base (38)..(38): 2′-O-methyladenosine
  modified_base (39)..(39): 2′-Fluoroadenosine
  modified_base (40)..(40): 2′-O-methylguanosine
  modified_base (41)..(41): 2′-Fluoroadenosine
  modified_base (42)..(42): 2′-O-methylguanosine
  modified_base (43)..(43): 2′-O-methyladenosine
  modified_base (44)..(44): 2′-O-methylcytidine, phosphorothioate
  modified_base (45)..(45): 2′-O-methyladenosine, phosphorothioate
  modified_base (46)..(46): 2′-O-methylguanosine

SEQ ID NO: 70 - RNA, 23 nt, Artificial Sequence
  Synthetic oligonucleotide
  modified_base (1)..(1): 2′-O-methyluridine, phosphorothioate
  modified_base (2)..(2): 2′-Fluorouridine, phosphorothioate
  modified_base (3)..(3): 2′-O-methylguanosine
  modified_base (4)..(4): 2′-O-methyluridine
  modified_base (5)..(5): 2′-O-methylguanosine
  modified_base (6)..(6): 2′-Fluorouridine
  modified_base (7)..(8): 2′-O-methyluridine
  modified_base (9)..(9): 2′-O-methylcytidine
  modified_base (10)..(11): 2′-O-methyladenosine
  modified_base (12)..(12): 2′-O-methylcytidine
  modified_base (13)..(13): 2′-O-methyluridine
  modified_base (14)..(14): 2′-Fluoroguanosine
  modified_base (15)..(15): 2′-O-methyluridine
  modified_base (16)..(16): 2′-Fluorouridine
  modified_base (17)..(17): 2′-O-methylcytidine
  modified_base (18)..(18): 2′-O-methyluridine
  modified_base (19)..(19): 2′-O-methylcytidine
  modified_base (20)..(20): 2′-O-methylguanosine
  modified_base (21)..(21): 2′-O-methyluridine, phosphorothioate
  modified_base (22)..(22): 2′-O-methylcytidine, phosphorothioate
  modified_base (23)..(23): 2′-O-methylguanosine
uuguguuucaacuguucucgucg

SEQ ID NO: 71 - RNA, 19 nt, Artificial Sequence
  Synthetic oligonucleotide
  modified_base (1)..(1): 2′-O-methylguanosine, phosphorothioate
  modified_base (2)..(2): 2′-O-methyluridine, phosphorothioate
  modified_base (3)..(3): 2′-O-methylcytidine
  modified_base (4)..(4): 2′-O-methyluridine
  modified_base (5)..(5): 2′-O-methylcytidine
  modified_base (6)..(6): 2′-O-methyluridine
  modified_base (7)..(7): 2′-Fluorouridine
  modified_base (8)..(8): 2′-O-methylguanosine
  modified_base (9)..(9): 2′-Fluoroguanosine
  modified_base (10)..(10): 2′-Fluoroadenosine
  modified_base (11)..(11): 2′-Fluorouridine
  modified_base (12)..(12): 2′-O-methyladenosine
  modified_base (13)..(14): 2′-O-methyluridine
  modified_base (15)..(15): 2′-O-methylcytidine
  modified_base (16)..(16): 2′-O-methyluridine
  modified_base (17)..(17): 2′-O-methylcytidine, phosphorothioate
  modified_base (18)..(18): 2′-O-methylguanosine, phosphorothioate
  modified_base (19)..(19): 2′-O-methyladenosine
gucucuuggauauucucga

SEQ ID NO: 72 - DNA, 46 bp, Artificial Sequence
  Synthetic oligonucleotide
  modified_base (1)..(1): 2′-O-methylguanosine, phosphorothioate
  modified_base (2)..(2): 2′-O-methyluridine, phosphorothioate
  modified_base (3)..(3): 2′-O-methylcytidine
  modified_base (4)..(4): 2′-O-methyluridine
  modified_base (5)..(5): 2′-O-methylcytidine
  modified_base (6)..(6): 2′-O-methyluridine
  modified_base (7)..(7): 2′-Fluorouridine
  modified_base (8)..(8): 2′-O-methylguanosine
  modified_base (9)..(9): 2′-Fluoroguanosine
  modified_base (10)..(10): 2′-Fluoroadenosine
  modified_base (11)..(11): 2′-Fluorouridine
  modified_base (12)..(12): 2′-O-methyladenosine
  modified_base (13)..(14): 2′-O-methyluridine
  modified_base (15)..(15): 2′-O-methylcytidine
  modified_base (16)..(16): 2′-O-methyluridine
  modified_base (17)..(17): 2′-O-methylcytidine, phosphorothioate
  modified_base (18)..(18): 2′-O-methylguanosine, phosphorothioate
  modified_base (19)..(19): 2′-O-methyladenosine
  modified_base (24)..(24): 2′-O-methyluridine, phosphorothioate
  modified_base (25)..(25): 2′-Fluorouridine, phosphorothioate
  modified_base (26)..(26): 2′-O-methylguanosine
  modified_base (27)..(27): 2′-O-methyluridine
  modified_base (28)..(28): 2′-O-methylguanosine
  modified_base (29)..(29): 2′-Fluorouridine
  modified_base (30)..(31): 2′-O-methyluridine
  modified_base (32)..(32): 2′-O-methylcytidine
  modified_base (33)..(34): 2′-O-methyladenosine
  modified_base (35)..(35): 2′-O-methylcytidine
  modified_base (36)..(36): 2′-O-methyluridine
  modified_base (37)..(37): 2′-Fluoroguanosine
  modified_base (38)..(38): 2′-O-methyluridine
  modified_base (39)..(39): 2′-Fluorouridine
  modified_base (40)..(40): 2′-O-methylcytidine
  modified_base (41)..(41): 2′-O-methyluridine
  modified_base (42)..(42): 2′-O-methylcytidine
  modified_base (43)..(43): 2′-O-methylguanosine
  modified_base (44)..(44): 2′-O-methyluridine, phosphorothioate
  modified_base (45)..(45): 2′-O-methylcytidine, phosphorothioate
  modified_base (46)..(46): 2′-O-methylguanosine
gucucuuggauauucucgattttuuguguuucaacuguucucgucg

SEQ ID NO: 73 - RNA, 21 nt, Artificial Sequence
  Synthetic oligonucleotide
  modified_base (1)..(1): 2′-O-methyluridine, phosphorothioate
  modified_base (2)..(2): 2′-Fluorocytidine, phosphorothioate
  modified_base (3)..(3): 2′-O-methylguanosine
  modified_base (4)..(4): 2′-O-methyladenosine
  modified_base (5)..(5): 2′-O-methylguanosine
  modified_base (6)..(6): 2′-Fluoroadenosine
  modified_base (7)..(7): 2′-O-methyladenosine
  modified_base (8)..(8): 2′-O-methyluridine
  modified_base (9)..(9): 2′-O-methyladenosine
  modified_base (10)..(10): 2′-O-methyluridine
  modified_base (11)..(12): 2′-O-methylcytidine
  modified_base (13)..(13): 2′-O-methyladenosine
  modified_base (14)..(14): 2′-Fluoroadenosine
  modified_base (15)..(15): 2′-O-methylguanosine
  modified_base (16)..(16): 2′-Fluoroadenosine
  modified_base (17)..(17): 2′-O-methylguanosine
  modified_base (18)..(18): 2′-O-methyladenosine
  modified_base (19)..(19): 2′-O-methylcytidine, phosphorothioate
  modified_base (20)..(20): 2′-O-methyladenosine, phosphorothioate
  modified_base (21)..(21): 2′-O-methylguanosine
ucgagaauauccaagagacag

SEQ ID NO: 74 - RNA, 21 nt, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(3): 2′-O-methylguanosine
  modified_base (4)..(4): 2′-O-methyladenosine
  modified_base (5)..(5): 2′-O-methylguanosine
  modified_base (6)..(6): 2′-O-methyladenosine
  modified_base (7)..(7): 2′-Fluoroadenosine
  modified_base (8)..(8): 2′-O-methylcytidine
  modified_base (9)..(9): 2′-Fluoroadenosine
  modified_base (10)..(10): 2′-Fluoroguanosine
  modified_base (11)..(11): 2′-Fluorouridine
  modified_base (12)..(12): 2′-O-methyluridine
  modified_base (13)..(13): 2′-O-methylguanosine
  modified_base (14)..(16): 2′-O-methyladenosine
  modified_base (17)..(17): 2′-O-methylcytidine
  modified_base (18)..(18): 2′-O-methyladenosine
  modified_base (19)..(19): 2′-O-methylcytidine, phosphorothioate
  modified_base (20)..(20): 2′-O-methyladenosine, phosphorothioate
  modified_base (21)..(21): 2′-O-methyladenosine
acgagaacaguugaaacacaa

SEQ ID NO: 75 - DNA, 48 bp, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(3): 2′-O-methylguanosine
  modified_base (4)..(4): 2′-O-methyladenosine
  modified_base (5)..(5): 2′-O-methylguanosine
  modified_base (6)..(6): 2′-O-methyladenosine
  modified_base (7)..(7): 2′-Fluoroadenosine
  modified_base (8)..(8): 2′-O-methylcytidine
  modified_base (9)..(9): 2′-Fluoroadenosine
  modified_base (10)..(10): 2′-Fluoroguanosine
  modified_base (11)..(11): 2′-Fluorouridine
  modified_base (12)..(12): 2′-O-methyluridine
  modified_base (13)..(13): 2′-O-methylguanosine
  modified_base (14)..(16): 2′-O-methyladenosine
  modified_base (17)..(17): 2′-O-methylcytidine
  modified_base (18)..(18): 2′-O-methyladenosine
  modified_base (19)..(20): 2′-O-methyladenosine, phosphorothioate
  modified_base (21)..(21): 2′-O-methyladenosine
  modified_base (26)..(26): 2′-O-methylguanosine, phosphorothioate
  modified_base (27)..(27): 2′-Fluorocytidine, phosphorothioate
  modified_base (28)..(28): 2′-O-methylcytidine
  modified_base (29)..(30): 2′-O-methyladenosine
  modified_base (31)..(31): 2′-Fluorocytidine
  modified_base (32)..(32): 2′-O-methyladenosine
  modified_base (33)..(33): 2′-O-methylguanosine
  modified_base (34)..(34): 2′-O-methylcytidine
  modified_base (35)..(35): 2′-O-methyluridine
  modified_base (36)..(37): 2′-O-methylcytidine
  modified_base (38)..(38): 2′-O-methyladenosine
  modified_base (39)..(39): 2′-Fluoroadenosine
  modified_base (40)..(40): 2′-O-methylcytidine
  modified_base (41)..(41): 2′-Fluorouridine
  modified_base (42)..(42): 2′-O-methyladenosine
  modified_base (43)..(44): 2′-O-methylcytidine
  modified_base (45)..(45): 2′-O-methyladenosine
  modified_base (46)..(46): 2′-O-methylcytidine, phosphorothioate
  modified_base (47)..(47): 2′-O-methyladenosine, phosphorothioate
  modified_base (48)..(48): 2′-O-methyladenosine
acgagaacaguugaaacaaaattttgccaacagcuccaacuaccacaa

SEQ ID NO: 76 - RNA, 23 nt, Artificial Sequence
  Synthetic oligonucleotide
  modified_base (1)..(1): 2′-O-methyluridine, phosphorothioate
  modified_base (2)..(2): 2′-Fluorouridine, phosphorothioate
  modified_base (3)..(3): 2′-O-methylguanosine
  modified_base (4)..(4): 2′-O-methyluridine
  modified_base (5)..(5): 2′-O-methylguanosine
  modified_base (6)..(6): 2′-Fluorouridine
  modified_base (7)..(8): 2′-O-methyluridine
  modified_base (9)..(9): 2′-O-methylcytidine
  modified_base (10)..(11): 2′-O-methyladenosine
  modified_base (12)..(12): 2′-O-methylcytidine
  modified_base (13)..(13): 2′-O-methyluridine
  modified_base (14)..(14): 2′-Fluoroguanosine
  modified_base (15)..(15): 2′-O-methyluridine
  modified_base (16)..(16): 2′-Fluorouridine
  modified_base (17)..(17): 2′-O-methylcytidine
  modified_base (18)..(18): 2′-O-methyluridine
  modified_base (19)..(19): 2′-O-methylcytidine
  modified_base (20)..(20): 2′-O-methylguanosine
  modified_base (21)..(21): 2′-O-methyluridine, phosphorothioate
  modified_base (22)..(22): 2′-O-methylcytidine, phosphorothioate
  modified_base (23)..(23): 2′-O-methylguanosine
uuguguuucaacuguucucgucg

SEQ ID NO: 77 - RNA, 21 nt, Artificial Sequence
  Synthetic oligonucleotide
  modified_base (1)..(1): 2′-O-methylguanosine, phosphorothioate
  modified_base (2)..(2): 2′-O-methyluridine, phosphorothioate
  modified_base (3)..(4): 2′-O-methylguanosine
  modified_base (5)..(5): 2′-O-methyluridine
  modified_base (6)..(6): 2′-O-methyladenosine
  modified_base (7)..(7): 2′-Fluoroguanosine
  modified_base (8)..(8): 2′-O-methyluridine
  modified_base (9)..(9): 2′-Fluorouridine
  modified_base (10)..(11): 2′-Fluoroguanosine
  modified_base (12)..(12): 2′-O-methyladenosine
  modified_base (13)..(13): 2′-O-methylguanosine
  modified_base (14)..(14): 2′-O-methylcytidine
  modified_base (15)..(15): 2′-O-methyluridine
  modified_base (16)..(16): 2′-O-methylguanosine
  modified_base (17)..(18): 2′-O-methyluridine
  modified_base (19)..(20): 2′-O-methylguanosine, phosphorothioate
  modified_base (21)..(21): 2′-O-methylcytidine
gugguaguuggagcuguuggc

SEQ ID NO: 78 - DNA, 46 bp, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(3): 2′-O-methylguanosine
  modified_base (4)..(4): 2′-O-methyladenosine
  modified_base (5)..(5): 2′-O-methylguanosine
  modified_base (6)..(6): 2′-O-methyladenosine
  modified_base (7)..(7): 2′-Fluoroadenosine
  modified_base (8)..(8): 2′-O-methylcytidine
  modified_base (9)..(9): 2′-Fluoroadenosine
  modified_base (10)..(10): 2′-Fluoroguanosine
  modified_base (11)..(11): 2′-Fluorouridine
  modified_base (12)..(12): 2′-O-methyluridine
  modified_base (13)..(13): 2′-O-methylguanosine
  modified_base (14)..(16): 2′-O-methyladenosine
  modified_base (17)..(17): 2′-O-methylcytidine
  modified_base (18)..(18): 2′-O-methyladenosine
  modified_base (19)..(19): 2′-O-methylcytidine, phosphorothioate
  modified_base (20)..(20): 2′-O-methyladenosine, phosphorothioate
  modified_base (21)..(21): 2′-O-methyladenosine
  modified_base (26)..(26): 2′-O-methyluridine, phosphorothioate
  modified_base (27)..(27): 2′-Fluoroadenosine, phosphorothioate
  modified_base (28)..(28): 2′-O-methylcytidine
  modified_base (29)..(30): 2′-O-methylguanosine
  modified_base (31)..(31): 2′-Fluorocytidine
  modified_base (32)..(32): 2′-O-methyladenosine
  modified_base (33)..(34): 2′-O-methylcytidine
  modified_base (35)..(35): 2′-O-methyladenosine
  modified_base (36)..(36): 2′-O-methylguanosine
  modified_base (37)..(37): 2′-O-methylcytidine
  modified_base (38)..(38): 2′-O-methyluridine
  modified_base (39)..(39): 2′-Fluorocytidine
  modified_base (40)..(40): 2′-O-methylcytidine
  modified_base (41)..(41): 2′-Fluoroadenosine
  modified_base (42)..(43): 2′-O-methyladenosine
  modified_base (44)..(44): 2′-O-methyluridine, phosphorothioate
  modified_base (45)..(45): 2′-O-methyladenosine, phosphorothioate
  modified_base (46)..(46): 2′-O-methylcytidine
acgagaacaguugaaacacaattttuacggcaccagcuccaaauac

SEQ ID NO: 79 - RNA, 23 nt, Artificial Sequence
  Synthetic oligonucleotide
  modified_base (1)..(1): 2′-O-methyluridine, phosphorothioate
  modified_base (2)..(2): 2′-Fluorouridine, phosphorothioate
  modified_base (3)..(3): 2′-O-methylguanosine
  modified_base (4)..(4): 2′-O-methyluridine
  modified_base (5)..(5): 2′-O-methylguanosine
  modified_base (6)..(6): 2′-Fluorouridine
  modified_base (7)..(8): 2′-O-methyluridine
  modified_base (9)..(9): 2′-O-methylcytidine
  modified_base (10)..(11): 2′-O-methyladenosine
  modified_base (12)..(12): 2′-O-methylcytidine
  modified_base (13)..(13): 2′-O-methyluridine
  modified_base (14)..(14): 2′-Fluoroguanosine
  modified_base (15)..(15): 2′-O-methyluridine
  modified_base (16)..(16): 2′-Fluorouridine
  modified_base (17)..(17): 2′-O-methylcytidine
  modified_base (18)..(18): 2′-O-methyluridine
  modified_base (19)..(19): 2′-O-methylcytidine
  modified_base (20)..(20): 2′-O-methylguanosine
  modified_base (21)..(21): 2′-O-methyluridine, phosphorothioate
  modified_base (22)..(22): 2′-O-methylcytidine, phosphorothioate
  modified_base (23)..(23): 2′-O-methylguanosine
uuguguuucaacuguucucgucg

SEQ ID NO: 80 - RNA, 19 nt, Artificial Sequence
  Synthetic oligonucleotide
  modified_base (1)..(1): 2′-O-methyladenosine, phosphorothioate
  modified_base (2)..(2): 2′-O-methylguanosine, phosphorothioate
  modified_base (3)..(4): 2′-O-methyluridine
  modified_base (5)..(6): 2′-O-methylguanosine
  modified_base (7)..(7): 2′-Fluoroadenosine
  modified_base (8)..(8): 2′-O-methylguanosine
  modified_base (9)..(9): 2′-Fluorocytidine
  modified_base (10)..(10): 2′-Fluorouridine
  modified_base (11)..(11): 2′-Fluoroguanosine
  modified_base (12)..(12): 2′-O-methylguanosine
  modified_base (13)..(13): 2′-O-methyluridine
  modified_base (14)..(14): 2′-O-methylguanosine
  modified_base (15)..(15): 2′-O-methyladenosine
  modified_base (16)..(16): 2′-O-methylcytidine
  modified_base (17)..(17): 2′-O-methylguanosine, phosphorothioate
  modified_base (18)..(18): 2′-O-methyluridine, phosphorothioate
  modified_base (19)..(19): 2′-O-methyladenosine
aguuggagcuggugacgua

SEQ ID NO: 81 - DNA, 44 bp, Artificial Sequence
  Synthetic oligonucleotide
  modified_base (1)..(1): 2′-O-methylcytidine, phosphorothioate
  modified_base (2)..(2): 2′-O-methyladenosine, phosphorothioate
  modified_base (3)..(3): 2′-O-methylguanosine
  modified_base (4)..(4): 2′-O-methylcytidine
  modified_base (5)..(5): 2′-O-methyluridine
  modified_base (6)..(6): 2′-O-methyladenosine
  modified_base (7)..(7): 2′-Fluoroadenosine
  modified_base (8)..(8): 2′-O-methyluridine
  modified_base (9)..(9): 2′-Fluorouridine
  modified_base (10)..(10): 2′-Fluorocytidine
  modified_base (11)..(11): 2′-Fluoroadenosine
  modified_base (12)..(12): 2′-O-methylguanosine
  modified_base (13)..(14): 2′-O-methyladenosine
  modified_base (15)..(15): 2′-O-methyluridine
  modified_base (16)..(16): 2′-O-methylcytidine
  modified_base (17)..(17): 2′-O-methyladenosine, phosphorothioate
  modified_base (18)..(18): 2′-O-methyluridine, phosphorothioate
  modified_base (19)..(19): 2′-O-methyluridine
  modified_base (24)..(24): 2′-O-methyluridine, phosphorothioate
  modified_base (25)..(25): 2′-Fluorocytidine, phosphorothioate
  modified_base (26)..(26): 2′-O-methylguanosine
  modified_base (27)..(27): 2′-O-methyladenosine
  modified_base (28)..(28): 2′-O-methylguanosine
  modified_base (29)..(29): 2′-Fluoroadenosine
  modified_base (30)..(30): 2′-O-methyladenosine
  modified_base (31)..(31): 2′-O-methyluridine
  modified_base (32)..(32): 2′-O-methyladenosine
  modified_base (33)..(33): 2′-O-methyluridine
  modified_base (34)..(35): 2′-O-methylcytidine
  modified_base (36)..(36): 2′-O-methyladenosine
  modified_base (37)..(37): 2′-Fluoroadenosine
  modified_base (38)..(38): 2′-O-methylguanosine
  modified_base (39)..(39): 2′-Fluoroadenosine
  modified_base (40)..(40): 2′-O-methylguanosine
  modified_base (41)..(41): 2′-O-methyladenosine
  modified_base (42)..(42): 2′-O-methylcytidine, phosphorothioate
  modified_base (43)..(43): 2′-O-methyladenosine, phosphorothioate
  modified_base (44)..(44): 2′-O-methylguanosine
cagcuaauucagaaucauuttttucgagaauauccaagagacag

SEQ ID NO: 82 - RNA, 21 nt, Artificial Sequence
  Synthetic oligonucleotide
  modified_base (1)..(1): 2′-O-methyladenosine, phosphorothioate
  modified_base (2)..(2): 2′-Fluoroadenosine, phosphorothioate
  modified_base (3)..(3): 2′-O-methyluridine
  modified_base (4)..(4): 2′-O-methylguanosine
  modified_base (5)..(5): 2′-O-methyladenosine
  modified_base (6)..(6): 2′-Fluorouridine
  modified_base (7)..(7): 2′-O-methyluridine
  modified_base (8)..(8): 2′-O-methylcytidine
  modified_base (9)..(9): 2′-O-methyluridine
  modified_base (10)..(10): 2′-O-methylguanosine
  modified_base (11)..(12): 2′-O-methyladenosine
  modified_base (13)..(13): 2′-O-methyluridine
  modified_base (14)..(14): 2′-Fluorouridine
  modified_base (15)..(15): 2′-O-methyladenosine
  modified_base (16)..(16): 2′-Fluoroguanosine
  modified_base (17)..(17): 2′-O-methylcytidine
  modified_base (18)..(18): 2′-O-methyluridine
  modified_base (19)..(19): 2′-O-methylguanosine, phosphorothioate
  modified_base (20)..(20): 2′-O-methyluridine, phosphorothioate
  modified_base (21)..(21): 2′-O-methyladenosine
aaugauucugaauuagcugua

SEQ ID NO: 83 - RNA, 19 nt, Artificial Sequence
  Synthetic oligonucleotide
  modified_base (1)..(1): 2′-O-methylguanosine, phosphorothioate
  modified_base (2)..(2): 2′-O-methyluridine, phosphorothioate
  modified_base (3)..(3): 2′-O-methylcytidine
  modified_base (4)..(4): 2′-O-methyluridine
  modified_base (5)..(5): 2′-O-methylcytidine
  modified_base (6)..(6): 2′-O-methyluridine
  modified_base (7)..(7): 2′-Fluorouridine
  modified_base (8)..(8): 2′-O-methylguanosine
  modified_base (9)..(9): 2′-Fluoroguanosine
  modified_base (10)..(10): 2′-Fluoroadenosine
  modified_base (11)..(11): 2′-Fluorouridine
  modified_base (12)..(12): 2′-O-methyladenosine
  modified_base (13)..(14): 2′-O-methyluridine
  modified_base (15)..(15): 2′-O-methylcytidine
  modified_base (16)..(16): 2′-O-methyluridine
  modified_base (17)..(17): 2′-O-methylcytidine, phosphorothioate
  modified_base (18)..(18): 2′-O-methylguanosine, phosphorothioate
  modified_base (19)..(19): 2′-O-methyladenosine
gucucuuggauauucucga

SEQ ID NO: 84 - DNA, 48 bp, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(3): 2′-O-methylguanosine
  modified_base (4)..(4): 2′-O-methyladenosine
  modified_base (5)..(5): 2′-O-methylguanosine
  modified_base (6)..(6): 2′-O-methyladenosine
  modified_base (7)..(7): 2′-Fluoroadenosine
  modified_base (8)..(8): 2′-O-methylcytidine
  modified_base (9)..(9): 2′-Fluoroadenosine
  modified_base (10)..(10): 2′-Fluoroguanosine
  modified_base (11)..(11): 2′-Fluorouridine
  modified_base (12)..(12): 2′-O-methyluridine
  modified_base (13)..(13): 2′-O-methylguanosine
  modified_base (14)..(16): 2′-O-methyladenosine
  modified_base (17)..(17): 2′-O-methylcytidine
  modified_base (18)..(18): 2′-O-methyladenosine
  modified_base (19)..(19): 2′-O-methylcytidine, phosphorothioate
  modified_base (20)..(20): 2′-O-methyladenosine, phosphorothioate
  modified_base (21)..(21): 2′-O-methyladenosine
  modified_base (26)..(26): 2′-O-methyladenosine, phosphorothioate
  modified_base (27)..(27): 2′-Fluorouridine, phosphorothioate
  modified_base (28)..(29): 2′-O-methyluridine
  modified_base (30)..(30): 2′-O-methylcytidine
  modified_base (31)..(31): 2′-Fluorouridine
  modified_base (32)..(32): 2′-O-methyluridine
  modified_base (33)..(34): 2′-O-methylcytidine
  modified_base (35)..(35): 2′-O-methyluridine
  modified_base (36)..(36): 2′-O-methylcytidine
  modified_base (37)..(37): 2′-O-methyladenosine
  modified_base (38)..(38): 2′-O-methyluridine
  modified_base (39)..(39): 2′-Fluorocytidine
  modified_base (40)..(40): 2′-O-methyluridine
  modified_base (41)..(41): 2′-Fluorouridine
  modified_base (42)..(42): 2′-O-methylcytidine
  modified_base (43)..(44): 2′-O-methyluridine
  modified_base (45)..(45): 2′-O-methylguanosine
  modified_base (46)..(47): 2′-O-methyluridine, phosphorothioate
  modified_base (48)..(48): 2′-O-methylcytidine
acgagaacaguugaaacacaattttauuucuuccucaucuucuuguuc

SEQ ID NO: 85 - RNA, 23 nt, Artificial Sequence
  Synthetic oligonucleotide
  modified_base (1)..(1): 2′-O-methyluridine, phosphorothioate
  modified_base (2)..(2): 2′-Fluorouridine, phosphorothioate
  modified_base (3)..(3): 2′-O-methylguanosine
  modified_base (4)..(4): 2′-O-methyluridine
  modified_base (5)..(5): 2′-O-methylguanosine
  modified_base (6)..(6): 2′-Fluorouridine
  modified_base (7)..(8): 2′-O-methyluridine
  modified_base (9)..(9): 2′-O-methylcytidine
  modified_base (10)..(11): 2′-O-methyladenosine
  modified_base (12)..(12): 2′-O-methylcytidine
  modified_base (13)..(13): 2′-O-methyluridine
  modified_base (14)..(14): 2′-Fluoroguanosine
  modified_base (15)..(15): 2′-O-methyluridine
  modified_base (16)..(16): 2′-Fluorouridine
  modified_base (17)..(17): 2′-O-methylcytidine
  modified_base (18)..(18): 2′-O-methyluridine
  modified_base (19)..(19): 2′-O-methylcytidine
  modified_base (20)..(20): 2′-O-methylguanosine
  modified_base (21)..(21): 2′-O-methyluridine, phosphorothioate
  modified_base (22)..(22): 2′-O-methylcytidine, phosphorothioate
  modified_base (23)..(23): 2′-O-methylguanosine
uuguguuucaacuguucucgucg

SEQ ID NO: 86 - RNA, 21 nt, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(4): 2′-O-methyladenosine
  modified_base (5)..(5): 2′-O-methylguanosine
  modified_base (6)..(6): 2′-O-methyladenosine
  modified_base (7)..(7): 2′-Fluoroadenosine
  modified_base (8)..(8): 2′-O-methylguanosine
  modified_base (9)..(9): 2′-Fluoroadenosine
  modified_base (10)..(10): 2′-Fluorouridine
  modified_base (11)..(11): 2′-Fluoroguanosine
  modified_base (12)..(12): 2′-O-methyladenosine
  modified_base (13)..(14): 2′-O-methylguanosine
  modified_base (15)..(16): 2′-O-methyladenosine
  modified_base (17)..(17): 2′-O-methylguanosine
  modified_base (18)..(18): 2′-O-methyladenosine
  modified_base (19)..(20): 2′-O-methyladenosine, phosphorothioate
  modified_base (21)..(21): 2′-O-methyluridine
acaagaagaugaggaagaaau

SEQ ID NO: 87 - DNA, 48 bp, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(4): 2′-O-methyladenosine
  modified_base (5)..(5): 2′-O-methylguanosine
  modified_base (6)..(6): 2′-O-methyladenosine
  modified_base (7)..(7): 2′-Fluoroadenosine
  modified_base (8)..(8): 2′-O-methylguanosine
  modified_base (9)..(9): 2′-Fluoroadenosine
  modified_base (10)..(10): 2′-Fluorouridine
  modified_base (11)..(11): 2′-Fluoroguanosine
  modified_base (12)..(12): 2′-O-methyladenosine
  modified_base (13)..(14): 2′-O-methylguanosine
  modified_base (15)..(16): 2′-O-methyladenosine
  modified_base (17)..(17): 2′-O-methylguanosine
  modified_base (18)..(18): 2′-O-methyladenosine
  modified_base (19)..(20): 2′-O-methyladenosine, phosphorothioate
  modified_base (21)..(21): 2′-O-methyluridine
  modified_base (26)..(26): 2′-O-methyluridine, phosphorothioate
  modified_base (27)..(27): 2′-Fluorouridine, phosphorothioate
  modified_base (28)..(28): 2′-O-methylguanosine
  modified_base (29)..(29): 2′-O-methyluridine
  modified_base (30)..(30): 2′-O-methylguanosine
  modified_base (31)..(31): 2′-Fluorouridine
  modified_base (32)..(33): 2′-O-methyluridine
  modified_base (34)..(34): 2′-O-methylcytidine
  modified_base (35)..(36): 2′-O-methyladenosine
  modified_base (37)..(37): 2′-O-methylcytidine
  modified_base (38)..(38): 2′-O-methyluridine
  modified_base (39)..(39): 2′-Fluoroguanosine
  modified_base (40)..(40): 2′-O-methyluridine
  modified_base (41)..(41): 2′-Fluorouridine
  modified_base (42)..(42): 2′-O-methylcytidine
  modified_base (43)..(43): 2′-O-methyluridine
  modified_base (44)..(44): 2′-O-methylcytidine
  modified_base (45)..(45): 2′-O-methylguanosine
  modified_base (46)..(46): 2′-O-methyluridine, phosphorothioate
  modified_base (47)..(47): 2′-O-methylcytidine, phosphorothioate
  modified_base (48)..(48): 2′-O-methylguanosine
acaagaagaugaggaagaaauttttuuguguuucaacuguucucgucg

SEQ ID NO: 88 - RNA, 23 nt, Artificial Sequence
  Synthetic oligonucleotide
  modified_base (1)..(1): 2′-O-methyladenosine, phosphorothioate
  modified_base (2)..(2): 2′-Fluorouridine, phosphorothioate
  modified_base (3)..(4): 2′-O-methyluridine
  modified_base (5)..(5): 2′-O-methylcytidine
  modified_base (6)..(6): 2′-Fluorouridine
  modified_base (7)..(7): 2′-O-methyluridine
  modified_base (8)..(9): 2′-O-methylcytidine
  modified_base (10)..(10): 2′-O-methyluridine
  modified_base (11)..(11): 2′-O-methylcytidine
  modified_base (12)..(12): 2′-O-methyladenosine
  modified_base (13)..(13): 2′-O-methyluridine
  modified_base (14)..(14): 2′-Fluorocytidine
  modified_base (15)..(15): 2′-O-methyluridine
  modified_base (16)..(16): 2′-Fluorouridine
  modified_base (17)..(17): 2′-O-methylcytidine
  modified_base (18)..(19): 2′-O-methyluridine
  modified_base (20)..(20): 2′-O-methylguanosine
  modified_base (21)..(22): 2′-O-methyluridine, phosphorothioate
  modified_base (23)..(23): 2′-O-methylcytidine
auuucuuccucaucuucuuguuc

SEQ ID NO: 89 - RNA, 21 nt, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(3): 2′-O-methylguanosine
  modified_base (4)..(4): 2′-O-methyladenosine
  modified_base (5)..(5): 2′-O-methylguanosine
  modified_base (6)..(6): 2′-O-methyladenosine
  modified_base (7)..(7): 2′-Fluoroadenosine
  modified_base (8)..(8): 2′-O-methylcytidine
  modified_base (9)..(9): 2′-Fluoroadenosine
  modified_base (10)..(10): 2′-Fluoroguanosine
  modified_base (11)..(11): 2′-Fluorouridine
  modified_base (12)..(12): 2′-O-methyluridine
  modified_base (13)..(13): 2′-O-methylguanosine
  modified_base (14)..(16): 2′-O-methyladenosine
  modified_base (17)..(17): 2′-O-methylcytidine
  modified_base (18)..(18): 2′-O-methyladenosine
  modified_base (19)..(19): 2′-O-methylcytidine, phosphorothioate
  modified_base (20)..(20): 2′-O-methyladenosine, phosphorothioate
  modified_base (21)..(21): 2′-O-methyladenosine
acgagaacaguugaaacacaa

SEQ ID NO: 90 - DNA, 48 bp, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(3): 2′-O-methylguanosine
  modified_base (4)..(4): 2′-O-methyladenosine
  modified_base (5)..(5): 2′-O-methylguanosine
  modified_base (6)..(6): 2′-O-methyladenosine
  modified_base (7)..(7): 2′-Fluoroadenosine
  modified_base (8)..(8): 2′-O-methylcytidine
  modified_base (9)..(9): 2′-Fluoroadenosine
  modified_base (10)..(10): 2′-Fluoroguanosine
  modified_base (11)..(11): 2′-Fluorouridine
  modified_base (12)..(12): 2′-O-methyluridine
  modified_base (13)..(13): 2′-O-methylguanosine
  modified_base (14)..(16): 2′-O-methyladenosine
  modified_base (17)..(17): 2′-O-methylcytidine
  modified_base (18)..(18): 2′-O-methyladenosine
  modified_base (19)..(19): 2′-O-methylcytidine, phosphorothioate
  modified_base (20)..(20): 2′-O-methyladenosine, phosphorothioate
  modified_base (21)..(21): 2′-O-methyladenosine
  modified_base (26)..(26): 2′-O-methyladenosine, phosphorothioate
  modified_base (27)..(27): 2′-Fluorouridine, phosphorothioate
  modified_base (28)..(28): 2′-O-methyluridine
  modified_base (29)..(29): 2′-O-methyladenosine
  modified_base (30)..(30): 2′-O-methylcytidine
  modified_base (31)..(31): 2′-Fluorouridine
  modified_base (32)..(32): 2′-O-methyluridine
  modified_base (33)..(33): 2′-O-methyladenosine
  modified_base (34)..(34): 2′-O-methylguanosine
  modified_base (35)..(35): 2′-O-methyladenosine
  modified_base (36)..(36): 2′-O-methylcytidine
  modified_base (37)..(37): 2′-O-methyladenosine
  modified_base (38)..(38): 2′-O-methyluridine
  modified_base (39)..(39): 2′-Fluorouridine
  modified_base (40)..(40): 2′-O-methyladenosine
  modified_base (41)..(41): 2′-Fluorocytidine
  modified_base (42)..(42): 2′-O-methyluridine
  modified_base (43)..(43): 2′-O-methyladenosine
  modified_base (44)..(44): 2′-O-methylguanosine
  modified_base (45)..(45): 2′-O-methyladenosine
  modified_base (46)..(47): 2′-O-methyluridine, phosphorothioate
  modified_base (48)..(48): 2′-O-methylguanosine
acgagaacaguugaaacacaattttauuacuuagacauuacuagauug

SEQ ID NO: 91 - RNA, 23 nt, Artificial Sequence
  Sythetic oligonucleotide
  modified_base (1)..(1): 2′-O-methyluridine, phosphorothioate
  modified_base (2)..(2): 2′-Fluorouridine, phosphorothioate
  modified_base (3)..(3): 2′-O-methylguanosine
  modified_base (4)..(4): 2′-O-methyluridine
  modified_base (5)..(5): 2′-O-methylguanosine
  modified_base (6)..(6): 2′-Fluorouridine
  modified_base (7)..(8): 2′-O-methyluridine
  modified_base (9)..(9): 2′-O-methylcytidine
  modified_base (10)..(11): 2′-O-methyladenosine
  modified_base (12)..(12): 2′-O-methylcytidine
  modified_base (13)..(13): 2′-O-methyluridine
  modified_base (14)..(14): 2′-Fluoroguanosine
  modified_base (15)..(15): 2′-O-methyluridine
  modified_base (16)..(16): 2′-Fluorouridine
  modified_base (17)..(17): 2′-O-methylcytidine
  modified_base (18)..(18): 2′-O-methyluridine
  modified_base (19)..(19): 2′-O-methylcytidine
  modified_base (20)..(20): 2′-O-methylguanosine
  modified_base (21)..(21): 2′-O-methyluridine, phosphorothioate
  modified_base (22)..(22): 2′-O-methylcytidine, phosphorothioate
  modified_base (23)..(23): 2′-O-methylguanosine
uuguguuucaacuguucucgucg

SEQ ID NO: 92 - RNA, 21 nt, Artificial Sequence
  Synthetic oligonucleotide
  modified_base (1)..(1): 2′-O-methyladenosine, phosphorothioate
  modified_base (2)..(2): 2′-O-methyluridine, phosphorothioate
  modified_base (3)..(3): 2′-O-methylcytidine
  modified_base (4)..(4): 2′-O-methyluridine
  modified_base (5)..(5): 2′-O-methyladenosine
  modified_base (6)..(6): 2′-O-methylguanosine
  modified_base (7)..(7): 2′-Fluorouridine
  modified_base (8)..(8): 2′-O-methyladenosine
  modified_base (9)..(9): 2′-Fluoroadenosine
  modified_base (10)..(10): 2′-Fluorouridine
  modified_base (11)..(11): 2′-Fluoroguanosine
  modified_base (12)..(12): 2′-O-methyluridine
  modified_base (13)..(13): 2′-O-methylcytidine
  modified_base (14)..(14): 2′-O-methyluridine
  modified_base (15)..(16): 2′-O-methyladenosine
  modified_base (17)..(17): 2′-O-methylguanosine
  modified_base (18)..(18): 2′-O-methyluridine
  modified_base (19)..(20): 2′-O-methyladenosine, phosphorothioate
  modified_base (21)..(21): 2′-O-methyluridine
aucuaguaaugucuaaguaau

SEQ ID NO: 93 - DNA, 48 bp, Artificial Sequence
  Synthetic oligonucleotide
  modified_base (1)..(1): 2′-O-methyluridine, phosphorothioate
  modified_base (2)..(2): 2′-O-methyladenosine, phosphorothioate
  modified_base (3)..(3): 2′-O-methyladenosine
  modified_base (4)..(4): 2′-O-methylcytidine
  modified_base (5)..(6): 2′-O-methyladenosine
  modified_base (7)..(7): 2′-Fluorouridine
  modified_base (8)..(8): 2′-O-methylguanosine
  modified_base (9)..(10): 2′-Fluorouridine
  modified_base (11)..(11): 2′-Fluoroguanosine
  modified_base (12)..(12): 2′-O-methylcytidine
  modified_base (13)..(16): 2′-O-methyluridine
  modified_base (17)..(17): 2′-O-methylcytidine
  modified_base (18)..(18): 2′-O-methyladenosine
  modified_base (19)..(20): 2′-O-methyladenosine, phosphorothioate
  modified_base (21)..(21): 2′-O-methyladenosine
  modified_base (26)..(26): 2′-O-methyluridine, phosphorothioate
  modified_base (27)..(27): 2′-Fluoroguanosine, phosphorothioate
  modified_base (28)..(28): 2′-O-methyladenosine
  modified_base (29)..(29): 2′-O-methylcytidine
  modified_base (30)..(30): 2′-O-methyladenosine
  modified_base (31)..(31): 2′-Fluorouridine
  modified_base (32)..(34): 2′-O-methyluridine
  modified_base (35)..(35): 2′-O-methyladenosine
  modified_base (36)..(36): 2′-O-methylguanosine
  modified_base (37)..(37): 2′-O-methyladenosine
  modified_base (38)..(38): 2′-O-methylcytidine
  modified_base (39)..(39): 2′-Fluorouridine
  modified_base (40)..(40): 2′-O-methylguanosine
  modified_base (41)..(41): 2′-Fluorouridine
  modified_base (42)..(42): 2′-O-methyladenosine
  modified_base (43)..(43): 2′-O-methylcytidine
  modified_base (44)..(44): 2′-O-methyladenosine
  modified_base (45)..(45): 2′-O-methylguanosine
  modified_base (46)..(46): 2′-O-methyluridine, phosphorothioate
  modified_base (47)..(47): 2′-O-methylguanosine, phosphorothioate
  modified_base (48)..(48): 2′-O-methylguanosine
uaacaauguugcuuuucaaaattttugacauuuuagacuguacagugg

SEQ ID NO: 94 - RNA, 23 nt, Artificial Sequence
  Synthetic oligonucleotide
  modified_base (1)..(1): 2′-O-methyluridine, phosphorothioate
  modified_base (2)..(2): 2′-Fluorouridine, phosphorothioate
  modified_base (3)..(4): 2′-O-methyluridine
  modified_base (5)..(5): 2′-O-methyladenosine
  modified_base (5)..(5): 2′-O-methylguanosine
  modified_base (6)..(6): 2′-Fluoroadenosine
  modified_base (7)..(9): 2′-O-methyladenosine
  modified_base (10)..(10): 2′-O-methylguanosine
  modified_base (11)..(11): 2′-O-methylcytidine
  modified_base (12)..(13): 2′-O-methyladenosine
  modified_base (14)..(14): 2′-Fluorocytidine
  modified_base (15)..(15): 2′-O-methyladenosine
  modified_base (16)..(16): 2′-Fluorouridine
  modified_base (17)..(17): 2′-O-methyluridine
  modified_base (18)..(18): 2′-O-methylguanosine
  modified_base (19)..(20): 2′-O-methyluridine
  modified_base (21)..(21): 2′-O-methyladenosine, phosphorothioate
  modified_base (22)..(22): 2′-O-methylguanosine, phosphorothioate
  modified_base (23)..(23): 2′-O-methyluridine
uuuugaaaagcaacauuguuagu

SEQ ID NO: 95 - RNA, 21 nt, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(3): 2′-O-methyluridine
  modified_base (4)..(4): 2′-O-methylguanosine
  modified_base (5)..(5): 2′-O-methyluridine
  modified_base (6)..(6): 2′-O-methyladenosine
  modified_base (7)..(7): 2′-Fluorocytidine
  modified_base (8)..(8): 2′-O-methyladenosine
  modified_base (9)..(9): 2′-Fluoroguanosine
  modified_base (10)..(10): 2′-Fluorouridine
  modified_base (11)..(11): 2′-Fluorocytidine
  modified_base (12)..(12): 2′-O-methyluridine
  modified_base (13)..(16): 2′-O-methyladenosine
  modified_base (17)..(17): 2′-O-methyluridine
  modified_base (18)..(18): 2′-O-methylguanosine
  modified_base (19)..(19): 2′-O-methyluridine, phosphorothioate
  modified_base (20)..(20): 2′-O-methylcytidine, phosphorothioate
  modified_base (21)..(21): 2′-O-methyladenosine
acuguacagucuaaaauguca

SEQ ID NO: 96 - DNA, 50 bp, Artificial Sequence
  Synthetic oligonucleotide
  modified_base (1)..(1): 2′-O-methyluridine, phosphorothioate
  modified_base (2)..(2): 2′-Fluorouridine, phosphorothioate
  modified_base (3)..(4): 2′-O-methyluridine
  modified_base (5)..(5): 2′-O-methylguanosine
  modified_base (6)..(6): 2′-Fluoroadenosine
  modified_base (7)..(9): 2′-O-methyladenosine
  modified_base (10)..(10): 2′-O-methylguanosine
  modified_base (11)..(11): 2′-O-methylcytidine
  modified_base (12)..(13): 2′-O-methyladenosine
  modified_base (14)..(14): 2′-Fluorocytidine
  modified_base (15)..(15): 2′-O-methyladenosine
  modified_base (16)..(16): 2′-Fluorouridine
  modified_base (17)..(17): 2′-O-methyluridine
  modified_base (18)..(18): 2′-O-methylguanosine
  modified_base (19)..(20): 2′-O-methyluridine
  modified_base (21)..(21): 2′-O-methyladenosine, phosphorothioate
  modified_base (22)..(22): 2′-O-methylguanosine, phosphorothioate
  modified_base (23)..(23): 2′-O-methyluridine
  modified_base (28)..(28): 2′-O-methyluridine, phosphorothioate
  modified_base (29)..(29): 2′-Fluoroguanosine, phosphorothioate
  modified_base (30)..(30): 2′-O-methyladenosine
  modified_base (31)..(31): 2′-O-methylcytidine
  modified_base (32)..(32): 2′-O-methyladenosine
  modified_base (33)..(33): 2′-Fluorouridine
  modified_base (34)..(36): 2′-O-methyluridine
  modified_base (37)..(37): 2′-O-methyladenosine
  modified_base (38)..(38): 2′-O-methylguanosine
  modified_base (39)..(39): 2′-O-methyladenosine
  modified_base (40)..(40): 2′-O-methylcytidine
  modified_base (41)..(41): 2′-Fluorouridine
  modified_base (42)..(42): 2′-O-methylguanosine
  modified_base (43)..(43): 2′-Fluorouridine
  modified_base (44)..(44): 2′-O-methyladenosine
  modified_base (45)..(45): 2′-O-methylcytidine
  modified_base (46)..(46): 2′-O-methyladenosine
  modified_base (47)..(47): 2′-O-methylguanosine
  modified_base (48)..(48): 2′-O-methyluridine, phosphorothioate
  modified_base (49)..(49): 2′-O-methylguanosine, phosphorothioate
  modified_base (50)..(50): 2′-O-methylguanosine
uuuugaaaagcaacauuguuaguttttugacauuuuagacuguacagugg

SEQ ID NO: 97 - RNA, 21 nt, Artificial Sequence
  Synthetic oligonucleotide
  modified_base (1)..(1): 2′-O-methyluridine, phosphorothioate
  modified_base (2)..(2): 2′-O-methyladenosine, phosphorothioate
  modified_base (3)..(3): 2′-O-methyladenosine
  modified_base (5)..(6): 2′-O-methyladenosine
  modified_base (7)..(7): 2′-Fluorouridine
  modified_base (8)..(8): 2′-O-methylguanosine
  modified_base (9)..(10): 2′-Fluorouridine
  modified_base (11)..(11): 2′-Fluoroguanosine
  modified_base (12)..(12): 2′-O-methylcytidine
  modified_base (13)..(16): 2′-O-methyluridine
  modified_base (17)..(17): 2′-O-methylcytidine
  modified_base (18)..(18): 2′-O-methyladenosine
  modified_base (19)..(20): 2′-O-methyladenosine, phosphorothioate
  modified_base (21)..(21): 2′-O-methyladenosine
uaacaauguugcuuuucaaaa

SEQ ID NO: 98 - RNA, 21 nt, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(3): 2′-O-methyluridine
  modified_base (4)..(4): 2′-O-methylguanosine
  modified_base (5)..(5): 2′-O-methyluridine
  modified_base (6)..(6): 2′-O-methyladenosine
  modified_base (7)..(7): 2′-Fluorocytidine
  modified_base (8)..(8): 2′-O-methyladenosine
  modified_base (9)..(9): 2′-Fluoroguanosine
  modified_base (10)..(10): 2′-Fluorouridine
  modified_base (11)..(11): 2′-Fluorocytidine
  modified_base (12)..(12): 2′-O-methyluridine
  modified_base (13)..(16): 2′-O-methyladenosine
  modified_base (17)..(17): 2′-O-methyluridine
  modified_base (18)..(18): 2′-O-methylguanosine
  modified_base (19)..(19): 2′-O-methyluridine, phosphorothioate
  modified_base (20)..(20): 2′-O-methylcytidine, phosphorothioate
  modified_base (21)..(21): 2′-O-methyladenosine
acuguacagucuaaaauguca

SEQ ID NO: 99 - DNA, 48 bp, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(3): 2′-O-methyluridine
  modified_base (4)..(4): 2′-O-methylguanosine
  modified_base (5)..(5): 2′-O-methyluridine
  modified_base (6)..(6): 2′-O-methyladenosine
  modified_base (7)..(7): 2′-Fluorocytidine
  modified_base (8)..(8): 2′-O-methyladenosine
  modified_base (9)..(9): 2′-Fluoroguanosine
  modified_base (10)..(10): 2′-Fluorouridine
  modified_base (11)..(11): 2′-Fluorocytidine
  modified_base (12)..(12): 2′-O-methyluridine
  modified_base (13)..(16): 2′-O-methyladenosine
  modified_base (17)..(17): 2′-O-methyluridine
  modified_base (18)..(18): 2′-O-methylguanosine
  modified_base (19)..(19): 2′-O-methyluridine, phosphorothioate
  modified_base (20)..(20): 2′-O-methylcytidine, phosphorothioate
  modified_base (21)..(21): 2′-O-methyladenosine
  modified_base (26)..(26): 2′-O-methyluridine, phosphorothioate
  modified_base (27)..(27): 2′-Fluorouridine, phosphorothioate
  modified_base (28)..(29): 2′-O-methyluridine
  modified_base (30)..(30): 2′-O-methylguanosine
  modified_base (31)..(31): 2′-Fluoroadenosine
  modified_base (32)..(34): 2′-O-methyladenosine
  modified_base (35)..(35): 2′-O-methylguanosine
  modified_base (36)..(36): 2′-O-methylcytidine
  modified_base (37)..(38): 2′-O-methyladenosine
  modified_base (39)..(39): 2′-Fluorocytidine
  modified_base (40)..(40): 2′-O-methyladenosine
  modified_base (41)..(41): 2′-Fluorouridine
  modified_base (42)..(42): 2′-O-methyluridine
  modified_base (43)..(43): 2′-O-methylguanosine
  modified_base (44)..(45): 2′-O-methyluridine
  modified_base (46)..(46): 2′-O-methyladenosine, phosphorothioate
  modified_base (47)..(47): 2′-O-methylguanosine, phosphorothioate
  modified_base (48)..(48): 2′-O-methyluridine
acuguacagucuaaaaugucattttuuuugaaaagcaacauuguuagu

SEQ ID NO: 100 - RNA, 23 nt, Artificial Sequence
  Synthetic oligonucleotide
  modified_base (1)..(1): 2′-O-methyluridine, phosphorothioate
  modified_base (2)..(2): 2′-Fluoroguanosine, phosphorothioate
  modified_base (3)..(3): 2′-O-methyladenosine
  modified_base (4)..(4): 2′-O-methylcytidine
  modified_base (5)..(5): 2′-O-methyladenosine
  modified_base (6)..(6): 2′-Fluorouridine
  modified_base (7)..(9): 2′-O-methyluridine
  modified_base (10)..(10): 2′-O-methyladenosine
  modified_base (11)..(11): 2′-O-methylguanosine
  modified_base (12)..(12): 2′-O-methyladenosine
  modified_base (13)..(13): 2′-O-methylcytidine
  modified_base (14)..(14): 2′-Fluorouridine
  modified_base (15)..(15): 2′-O-methylguanosine
  modified_base (16)..(16): 2′-Fluorouridine
  modified_base (17)..(17): 2′-O-methyladenosine
  modified_base (18)..(18): 2′-O-methylcytidine
  modified_base (19)..(19): 2′-O-methyladenosine
  modified_base (20)..(20): 2′-O-methylguanosine
  modified_base (21)..(21): 2′-O-methyluridine, phosphorothioate
  modified_base (22)..(22): 2′-O-methylguanosine, phosphorothioate
  modified_base (23)..(23): 2′-O-methylguanosine
ugacauuuuagacuguacagugg

SEQ ID NO: 101 - RNA, 21 nt, Artificial Sequence
  Synthetic oligonucleotide
  modified_base (1)..(1): 2′-O-methyluridine, phosphorothioate
  modified_base (2)..(2): 2′-O-methyladenosine, phosphorothioate
  modified_base (3)..(3): 2′-O-methyladenosine
  modified_base (4)..(4): 2′-O-methylcytidine
  modified_base (5)..(6): 2′-O-methyladenosine
  modified_base (7)..(7): 2′-Fluorouridine
  modified_base (8)..(8): 2′-O-methylguanosine
  modified_base (9)..(10): 2′-Fluorouridine
  modified_base (11)..(11): 2′-Fluoroguanosine
  modified_base (12)..(12): 2′-O-methylcytidine
  modified_base (13)..(16): 2′-O-methyluridine
  modified_base (17)..(17): 2′-O-methylcytidine
  modified_base (18)..(18): 2′-O-methyladenosine
  modified_base (19)..(20): 2′-O-methyladenosine, phosphorothioate
  modified_base (21)..(21): 2′-O-methyladenosine
uaacaauguugcuuuucaaaa

SEQ ID NO: 102 - DNA, 50 bp, Artificial Sequence
  Synthetic oligonucleotide
  modified_base (1)..(1): 2′-O-methyluridine, phosphorothioate
  modified_base (2)..(2): 2′-Fluoroguanosine, phosphorothioate
  modified_base (3)..(3): 2′-O-methyladenosine
  modified_base (4)..(4): 2′-O-methylcytidine
  modified_base (5)..(5): 2′-O-methyladenosine
  modified_base (6)..(6): 2′-Fluorouridine
  modified_base (7)..(9): 2′-O-methyluridine
  modified_base (10)..(10): 2′-O-methyladenosine
  modified_base (11)..(11): 2′-O-methylguanosine
  modified_base (12)..(12): 2′-O-methyladenosine
  modified_base (13)..(13): 2′-O-methylcytidine
  modified_base (14)..(14): 2′-Fluorouridine
  modified_base (15)..(15): 2′-O-methylguanosine
  modified_base (16)..(16): 2′-Fluorouridine
  modified_base (17)..(17): 2′-O-methyladenosine
  modified_base (18)..(18): 2′-O-methylcytidine
  modified_base (19)..(19): 2′-O-methyladenosine
  modified_base (20)..(20): 2′-O-methylguanosine
  modified_base (21)..(21): 2′-O-methyluridine, phosphorothioate
  modified_base (22)..(22): 2′-O-methylguanosine, phosphorothioate
  modified_base (23)..(23): 2′-O-methylguanosine
  modified_base (28)..(28): 2′-O-methyluridine, phosphorothioate
  modified_base (29)..(29): 2′-Fluorouridine, phosphorothioate
  modified_base (30)..(31): 2′-O-methyluridine
  modified_base (32)..(32): 2′-O-methylguanosine
  modified_base (33)..(33): 2′-Fluoroadenosine
  modified_base (34)..(36): 2′-O-methyladenosine
  modified_base (37)..(37): 2′-O-methylguanosine
  modified_base (38)..(38): 2′-O-methylcytidine
  modified_base (39)..(40): 2′-O-methyladenosine
  modified_base (41)..(41): 2′-Fluorocytidine
  modified_base (42)..(42): 2′-O-methyladenosine
  modified_base (43)..(43): 2′-Fluorouridine
  modified_base (44)..(44): 2′-O-methyluridine
  modified_base (45)..(45): 2′-O-methylguanosine
  modified_base (46)..(47): 2′-O-methyluridine
  modified_base (48)..(48): 2′-O-methyladenosine, phosphorothioate
  modified_base (49)..(49): 2′-O-methylguanosine, phosphorothioate
  modified_base (50)..(50): 2′-O-methyluridine
ugacauuuuagacuguacaguggttttuuuugaaaagcaacauuguuagu

SEQ ID NO: 103 - RNA, 21 nt, Artificial Sequence
  Synthetic oligonucleotide
  modified_base (1)..(1): 2′-O-methyladenosine, phosphorothioate
  modified_base (2)..(2): 2′-O-methylcytidine, phosphorothioate
  modified_base (3)..(3): 2′-O-methyluridine
  modified_base (4)..(4): 2′-O-methylguanosine
  modified_base (5)..(5): 2′-O-methyluridine
  modified_base (6)..(6): 2′-O-methyladenosine
  modified_base (7)..(7): 2′-Fluorocytidine
  modified_base (8)..(8): 2′-O-methyladenosine
  modified_base (9)..(9): 2′-Fluoroguanosine
  modified_base (10)..(10): 2′-Fluorouridine
  modified_base (11)..(11): 2′-Fluorocytidine
  modified_base (12)..(12): 2′-O-methyluridine
  modified_base (13)..(16): 2′-O-methyladenosine
  modified_base (17)..(17): 2′-O-methyluridine
  modified_base (18)..(18): 2′-O-methylguanosine
  modified_base (19)..(19): 2′-O-methyluridine, phosphorothioate
  modified_base (20)..(20): 2′-O-methylcytidine, phosphorothioate
  modified_base (21)..(21): 2′-O-methyladenosine
acuguacagucuaaaauguca

SEQ ID NO: 104 - RNA, 21 nt, Artificial Sequence
  Synthetic oligonucleotide
  modified_base (1)..(1): 2′-O-methyluridine, phosphorothioate
  modified_base (2)..(2): 2′-O-methyladenosine, phosphorothioate
  modified_base (3)..(3): 2′-O-methyladenosine
  modified_base (4)..(4): 2′-O-methylcytidine
  modified_base (5)..(6): 2′-O-methyladenosine
  modified_base (7)..(7): 2′-Fluorouridine
  modified_base (8)..(8): 2′-O-methylguanosine
  modified_base (9)..(10): 2′-Fluorouridine
  modified_base (11)..(11): 2′-Fluoroguanosine
  modified_base (12)..(12): 2′-O-methylcytidine
  modified_base (13)..(16): 2′-O-methyluridine
  modified_base (17)..(17): 2′-O-methylcytidine
  modified_base (18)..(18): 2′-O-methyladenosine
  modified_base (19)..(20): 2′-O-methyladenosine, phosphorothioate
  modified_base (21)..(21): 2′-O-methyladenosine
uaacaauguugcuuuucaaaa

SEQ ID NO: 105 - RNA, 21 nt, Artificial Sequence
  Synthetic oligonucleotide
  modified_base (1)..(1): 2′-Fluoroguanosine, phosphorothioate
  modified_base (2)..(2): 2′-O-methyluridine, phosphorothioate
  modified_base (3)..(3): 2′-Fluoroadenosine
  modified_base (4)..(4): 2′-O-methylguanosine
  modified_base (5)..(5): 2′-Fluorouridine
  modified_base (6)..(6): 2′-O-methyluridine
  modified_base (7)..(7): 2′-Fluoroguanosine
  modified_base (8)..(8): 2′-O-methylguanosine
  modified_base (9)..(9): 2′-Fluoroadenosine
  modified_base (10)..(10): 2′-Fluoroguanosine
  modified_base (11)..(11): 2′-Fluorocytidine
  modified_base (12)..(12): 2′-O-methyluridine
  modified_base (13)..(13): 2′-Fluoroguanosine
  modified_base (14)..(14): 2′-O-methyluridine
  modified_base (15)..(15): 2′-Fluorouridine
  modified_base (16)..(16): 2′-O-methylguanosine
  modified_base (17)..(17): 2′-Fluoroguanosine
  modified_base (18)..(18): 2′-O-methylcytidine
  modified_base (19)..(19): 2′-Fluoroguanosine
  modified_base (20)..(20): 2′-O-methyluridine
  modified_base (21)..(21): 2′-Fluoroadenosine
guaguuggagcuguuggcgua

SEQ ID NO: 106 - RNA, 23 nt, Artificial Sequence
  Synthetic oligonucleotide
  modified_base (1)..(1): 2′-O-methyluridine, phosphorothioate
  modified_base (2)..(2): 2′-Fluoroadenosine, phosphorothioate
  modified_base (3)..(3): 2′-O-methylcytidine
  modified_base (4)..(4): 2′-Fluoroguanosine
  modified_base (5)..(5): 2′-O-methylcytidine
  modified_base (6)..(6): 2′-Fluorocytidine
  modified_base (7)..(7): 2′-O-methyladenosine
  modified_base (8)..(8): 2′-Fluoroadenosine
  modified_base (9)..(9): 2′-O-methylcytidine
  modified_base (10)..(10): 2′-Fluoroadenosine
  modified_base (11)..(11): 2′-O-methylguanosine
  modified_base (12)..(12): 2′-O-methylcytidine
  modified_base (13)..(13): 2′-O-methyluridine
  modified_base (14)..(14): 2′-Fluorocytidine
  modified_base (15)..(15): 2′-O-methylcytidine
  modified_base (16)..(16): 2′-Fluoroadenosine
  modified_base (17)..(17): 2′-O-methyladenosine
  modified_base (18)..(18): 2′-Fluorocytidine
  modified_base (19)..(19): 2′-O-methyluridine
  modified_base (20)..(20): 2′-Fluoroadenosine
  modified_base (21)..(22): 2′-O-methylcytidine, phosphorothioate
  modified_base (23)..(23): 2′-O-methyladenosine
uacgccaacagcuccaacuacca